(19) United States
(12) Patent Application Publication
(10) Pub. No.: US 2005/0107349 A1
(43) Pub. Date: May 19, 2005

(54) Method for the treatment or prevention of respiratory disorders with a cyclooxygenase-2 inhibitor in combination with a muscarinic receptor antagonist and compositions therewith

(75) Inventor: Karen Seibert, St. Louis, MO (US)

Correspondence Address: James E. Davis;Harness, Dickey & Pierce, P. L. C., 7700 Bonhomme, Suite 400, Clayton, MO, 63105, US

(73) Assignee: Pharmacia Corporation, Chesterfield, MO (US)

(21) Appl. No.: 10/833,658

(22) Filed: Apr. 28, 2004

Related U.S. Application Data
(60) Provisional application No. 60/489,966, filed on Jul. 24, 2003.

(51) Int. Cl. A61K 31/60; A61K 31/415; A61K 31/192

(52) U.S. Cl. 514/165; 514/406; 514/569; 514/570; 514/420

(57) ABSTRACT

The present invention relates to a novel method of preventing and/or treating respiratory disorders and respiratory disorder-related complications in a subject by administering to the subject at least one Cox-2 inhibitor in combination with one or more muscarinic receptor antagonists. Compositions, pharmaceutical compositions and kits are also described.

### CROSS-REFERENCE TO RELATED PATENTS AND PATENT APPLICATIONS

This application is related to and claims the priority benefit of U.S. patent application Ser. No. 60/489,966 filed Jul. 24, 2003, which is incorporated by reference herein in its entirety.

### BACKGROUND OF THE INVENTION

1) Field of the Invention

The present invention relates generally to the use of an enzyme inhibitor and an enzyme antagonist in combination for the treatment or prevention of respiratory disorders, and in particular to the use of a cyclooxygenase-2 inhibitor in combination with a muscarinic receptor antagonist.

2) Description of the Related Art

A broad spectrum of respiratory diseases and disorders has been recognized, many of which have overlapping and interacting etiologies. Two of the most widespread and prevalent of these diseases are chronic obstructive pulmonary disorder (COPD) and asthma.

COPD is a chronic respiratory disorder characterized by airflow limitation, accompanied by shortness of breath, cough, wheezing, increased sputum production and occasionally fever. See The Merck Manual of Diagnosis & Therapy, Beers & Brakow, 17^{th} edition, Published by Merck Research Labs, Sec. 6, Chapter 68, Chronic Obstructive Airway Disorders, COPD (1999). Many factors contribute to the risk of developing COPD, including breathing heavy dust, air pollution, poor nutrition, childhood respiratory infections, chronic uncontrolled asthma, and even heredity. Nevertheless, almost 90% of COPD cases are caused by long-term cigarette smoking and/or passive exposure to cigarette smoke.

Cigarette smoke contains an abundance of toxic and irritating substances. Over time, cigarette smoke produces inflammation in both the bronchial tubes of the lungs and the walls of the alveoli. In the alveoli, smoke-induced inflammatory cells destroy the capillaries and air sacs, giving rise to permanent lung damage or emphysema. In addition, cigarette smoke induces inflammation in the airways and causes swelling which reduces the diameter of these passages. The inflammation in the bronchi also produces large amounts of mucus. The swelling of the bronchial tubes, the increased mucus production, and bronchial muscle spasm can obstruct airflow into and out of the lungs; all leading to COPD.

COPD is characterized by a reduction of airflow from the lungs and an increase in air trapped inside the lungs. As more air is trapped in the lungs, air exchange becomes progressively more limited. Significant airflow obstruction makes the work of breathing more difficult and affects the lung's ability to get oxygen into the blood and remove carbon dioxide from the body. Meanwhile, damage to pulmonary blood vessels places a growing burden on the heart.

COPD progresses with age, eventually leading to cardiovascular and/or respiratory failure and early death. According to the Annual World Health Report of the World Health Organization (WHO), about 600 million people suffer from COPD, with some three million dying from the disease each year. A primary goal in treating subjects suffering from COPD is to increase airflow, reduce inflammation and, consequently, improve their ability to participate in daily activities.

Asthma is a pulmonary disease that is characterized by reversible airway obstruction, airway inflammation, and increased airway responsiveness (manifested as bronchoconstriction), due to a variety of irritating stimuli. Airway obstruction in asthma is due to a combination of factors including spasm of airway smooth muscle, edema of airway mucosa, increased mucus secretion, and cellular infiltration of the airway walls. Symptoms of asthma usually begin quite suddenly with wheezing episodes, coughing and shortness of breath.

Bronchoconstriction is the primary symptom of many respiratory disorders, including, for example, COPD and asthma. Bronchoconstriction is an airflow limitation resulting from contraction of the smooth muscle that envelops the bronchi and bronchioles. This airway contraction makes it very difficult for air to pass through the lungs and can lead to symptoms of wheezing, coughing, tightness of the chest, and breathlessness as a subject suffering from such a symptom tries to breathe.

When the mucosa lining the airway passages is thickened by inflammation from an existing pulmonary disorder, even a minor smooth muscle contraction can substantially narrow the airways and make breathing more difficult. Airflow is also compromised by the increased production of mucus in response to inhaled irritants. Accumulated mucus and cellular debris in the airways further constricts the diameter of the air passages and increases the effort required to breathe.

Bronchoconstriction is thought to occur when inhaled irritants, such as cigarette smoke, stimulate sensory nerve endings called irritant receptors lying below the airway epithelium. Stimulation of these irritant receptors causes parasympathetic nerves to release acetylcholine (ACh). The binding of acetylcholine to muscarinic receptors located on airway smooth muscle cells causes bronchoconstriction (bronchospasm), which is a safety mechanism that prevents irritants from penetrating even deeper into the lungs. Thus, anticholinergics or, more specifically, muscarinic receptor antagonists, are effective in reducing smooth muscle contractions in the bronchi and bronchioles that causes constriction of the airways.

Recently, it has been discovered that there are at least five different muscarinic receptor subtypes localized within lung and airway tissue, namely subtypes M₁, M₂, M₃, M₄ and M₅. The M₁ and M₃ subtypes mediate bronchoconstriction by promoting transfer of the action potential down from the autonomic nervous system to airway smooth muscle. The M₂ subtype, in contrast, appears to be an auto-regulator that turns off further secretion of acetylcholine from the nerve terminal. Muscarinic receptor antagonists can reduce bronchoconstriction by binding to muscarinic receptors and thus, block the binding of acetylcholine. Blocking the effects of acetylcholine binding to muscarinic receptors in the lungs results in relaxation of the muscles around the bronchi, and thus, allows for easier breathing by a subject suffering from a respiratory disorder characterized by bronchoconstriction.

Non-selective antagonism of all muscarinic subtypes results in different outcomes versus selective antagonism of particular subtypes. Agonism of the M₁ and M₃ receptors is responsible for cholinergic induced bronchoconstriction. In contrast, agonism of the M₂ receptor plays a role in a negative feedback loop that serves to inhibit further cholinergic activity. Thus, the majority of the bronchodilator effects of anticholinergics are mediated by selective antagonism of the M₁ and M₃ receptors, while blockade of M₂ receptors results in increased acetylcholine release, which is not only unhelpful for bronchodilation, but also has unwanted side effects.

Of concern is that a treatment therapy comprising a muscarinic receptor antagonist alone may not produce the systemic reduction in respiratory symptoms that is currently desired. For example, many reports disclose that the effectiveness of muscarinic receptor antagonists are dependent upon which subtype of muscarinic receptor is inhibited.

Currently available muscarinic receptor antagonists are nonselective in their mode of action and include such compounds as ipratropium bromide (Atrovent®)). Ipratropium is non-selective with respect to the muscarinic receptor subtypes that it blocks or antagonizes, thus, it inhibits all of them. Such non-selective antagonists have the disadvantage of several side effects, including nervousness, dizziness, headache, nausea, mydriasis, upset stomach, dry mouth, throat irritation, and cough. Despite this, ipratropium bromide is presently a leading respiratory therapeutic. For these reasons, the ipratropium bromide class of compounds could be greatly improved by the development of agents with improved selectivity (for M₁ and M₃ receptors) and/or an extended duration of action.

Unfortunately, the treatment of respiratory disorders with muscarinic receptor antagonists, such as ipratropium bromide, alone fails to address the underlying inflammation. This is problematic because many pulmonary disorders are thought to arise, in part, from the release of inflammatory mediators formed within the lungs. For example, many pulmonary disorders cause the pulmonary airway wall to undergo a reaction resulting in the infiltration of a variety of inflammatory cells such as eosinophils, mast cells, and CD4+ T-lymphocytes. See Bundschuh, D., et al., Pharm. Exper. Therap., 297(1):280-290 (2001). These inflammatory cells can release a plethora of mediators, including histamine and the products of arachidonic acid metabolism, such as leukotrienes and prostaglandins, cytokines, interleukins IL-1 to IL-12, alpha-, beta- and gamma-interferon, tumor necrosis factor (TNF) and proteases, all ultimately leading to several harmful symptoms including inflammation and bronchoconstriction.

Typical of the development of many inflammatory symptoms is upregulation of the enzyme, cyclooxygenase-2 (Cox-2). Cox-2 is an enzyme produced by an inducible gene, which is responsible for the biosynthesis of prostaglandins in inflammatory cells. Inflammation causes the induction of the Cox-2 enzyme, leading to the release of prostanoids (prostaglandin E2), which sensitize peripheral nociceptor terminals and produce localized inflammation and edema. See e.g., Samad, T., et al., Nature 410(6827):471-5 (2001).

Historically, physicians have treated inflammation-related disorders with a regimen of nonsteroidal anti-inflammatory drugs (NSAIDs), such as, for example, aspirin and ibuprofen. Undesirably, however, some NSAIDs were known to cause gastrointestinal (GI) bleeding or ulcers in subjects undergoing consistent long-term regimens of NSAID therapy.

A reduction of unwanted side effects of common NSAIDs was made possible by the discovery that two cyclooxygenases are involved in the transformation of arachidonic acid as the first step in the prostaglandin synthesis pathway. These enzymes exist in two forms and have been termed cyclooxygenase-1 (Cox-1) and Cox-2. See Needleman, P., et al., J. Rheumatol. 24, Suppl. 49:6-8 (1997).

Cox-1 is a constitutive enzyme responsible for the biosynthesis of prostaglandins in the gastric mucosa and in the kidney. Many common NSAIDs are now known to be inhibitors of both Cox-1 and Cox-2. Accordingly, when administered in sufficiently high levels, these NSAIDs not only alleviate the inflammatory consequences of Cox-2 activity, but also inhibit the beneficial gastric maintenance activities of Cox-1.

While the beneficial effects of Cox-2 inhibitors on inflammation and inflammation-related disorders have been recognized, the effects of Cox-2 inhibitors on respiratory diseases and disorders have not been as widely reported. In fact, certain cyclooxygenase inhibitors have been implicated as a causative agent in at least one respiratory disorder, including, for example asthma attacks. See Martin-Garcia, C., et al., Chest, 121(6):1812-1817 (2002). In light of such reports, one would not expect Cox-2 inhibitors to show efficacy in treating respiratory disorders, especially in combination with muscarinic receptor antagonists. Moreover, an effective combination therapy comprising a muscarinic receptor antagonist and a Cox-2 inhibitor has not been reported until now.

Despite the recent advances that have been made in understanding the causes of respiratory disorders, they remain notoriously difficult to treat. From the foregoing, it can be seen that a need exists for improved methods and therapeutic compositions for the prevention and treatment of respiratory disorders such as COPD and asthma. It would also be useful to provide an improved method and composition for reducing both the inflammation and bronchoconstriction associated with respiratory disorders. Likewise, methods and compositions that improve patient airway responses following acute respiratory episodes would also be desirable. Also, methods and compositions that reduce dosages or reduce unwanted side effects in conventional treatments for respiratory disorders are desirable. Finally, methods and compositions that improve the efficacy of treating a respiratory disorder that is considered resistant or intractable to known methods of therapy alone would also be desirable.

### SUMMARY OF THE INVENTION

Briefly, therefore, the present invention is directed to a novel method for preventing or treating a respiratory disorder in a subject comprising administering to the subject a Cox-2 inhibitor in combination with a muscarinic receptor antagonist.

The present invention is also directed to a novel method for preventing or treating a respiratory disorder in a subject that is in need of such prevention or treatment comprising administering to the subject a Cox-2 inhibitor in combination with a muscarinic receptor antagonist.

The present invention is also directed to a novel therapeutic composition comprising a Cox-2 inhibitor and a muscarinic receptor antagonist.

The present invention is also directed to a novel pharmaceutical composition comprising a Cox-2 inhibitor, a muscarinic receptor antagonist, and a pharmaceutically acceptable carrier.

The present invention is also directed to a kit comprising one dosage form comprising a Cox-2 inhibitor and a second dosage form comprising a muscarinic receptor antagonist.

The present invention is also directed to a novel method of preventing or treating a pathological condition or physiological disorder characterized by or associated with lung inflammation and bronchoconstriction in a subject that is in need of such therapy comprising administering to the subject a Cox-2 inhibitor and a muscarinic receptor modulating amount of a muscarinic receptor antagonist.

Among the several advantages found to be achieved by the present invention, therefore, may be noted the provision of improved methods and therapeutic compositions for the prevention or treatment of respiratory disorders such as COPD and asthma. Other advantages achieved by the present invention include improved methods and compositions for reducing both the inflammation and bronchoconstriction associated with respiratory disorders. Still other advantages achieved by the present invention include methods and compositions that improve patient airway responses following acute respiratory episodes. In addition, the present invention provides methods and compositions that reduce dosages or reduce unwanted side effects in conventional treatments for respiratory disorders are desirable. Finally, the present invention provides methods and compositions that improve the efficacy of treating a respiratory disorder that is considered resistant or intractable to known methods of therapy alone.

### DETAILED DESCRIPTION OF THE PREFERRED EMBODIMENTS

In accordance with the present invention it has been discovered that the treatment or prevention of respiratory disorders, including such disorders as COPD and asthma, is provided by a combination therapy comprising a Cox-2 inhibitor and a muscarinic receptor antagonist.

For purposes of the present invention, the novel combination therapy comprising at least one Cox-2 inhibitor in combination with one or more muscarinic receptor antagonists is useful for the purpose of preventing and treating respiratory disorders and respiratory disorder-related complications in a subject and, in preferred embodiments, the method is useful for the purpose of preventing and treating respiratory disorders and respiratory disorder-related complications in a subject that is in need of such prevention and treatment.

The administration of a combination of a Cox-2 inhibitor and a muscarinic receptor antagonist for preventing or treating a respiratory disorder to a subject in need of such therapy is superior to the use of either compound alone. In fact, for preferred embodiments of the invention, it is believed that the results provided by the combination of a Cox-2 inhibitor and a muscarinic receptor antagonist in preventing or treating a respiratory disorder are superior to the results that would be expected of the combination on the basis of the results provided by use of the Cox-2 inhibitor or the muscarinic receptor antagonist separately. The combination therapy of the invention would be useful, for example, to reduce the death rate or the number of non-fatal hospitalizations, or to prevent or retard the development of COPD, which can arise from chronic cigarette smoking.

The combination therapy of the present invention would be useful, for example, to reduce such respiratory disorder symptoms as, for example, coughing, inflammation, congestion, dyspnea, wheezing, hyperventilation, difficulty breathing, bronchospasm, and bronchoconstriction in a subject suffering from such symptoms. The combination therapy of the present invention would also be useful to prevent the occurrence of such symptoms.

The methods and compositions of the present invention are also useful to reduce the number of hospitalizations of subjects suffering from a chronic respiratory disorder, or to prevent or retard, in subjects, the development of complications associated with respiratory disorders, such as, for example, COPD, lung abscesses or respiratory failure, which may eventually arise from having a chronic or recurring respiratory disorder.

Furthermore, the administration of a Cox-2 inhibitor in combination with a muscarinic receptor antagonist is an effective treatment for respiratory disorders or respiratory disorder-related complications, and in preferred embodiments, is unexpectedly superior to the use of either agent alone.

The administration of a Cox-2 inhibitor in combination with a muscarinic receptor antagonist for the prevention and treatment of respiratory disorders and respiratory disorder-related complications is an unexpectedly effective treatment and preventative therapy. Such administration is effective for improving the symptoms of respiratory disorders and respiratory disorder-related complications while avoiding or reducing certain disadvantages of current treatments. The combination therapy of a Cox-2 inhibitor and a muscarinic receptor antagonist is also useful for decreasing the required number of separate dosages, thus, potentially improving patient compliance.

Combination therapies comprising Cox-2 inhibitors and muscarinic receptor antagonists are useful not only for improving respiratory disorder symptoms and shortening recovery times, but also for reducing the dosages of conventional muscarinic receptor antagonists that are normally required. The administration of lower dosages of muscarinic receptor antagonists provides a reduction in side effects corresponding to such muscarinic receptor antagonists.

As used herein, the phrases “combination therapy”, “co-administration”, “co-administering”, “administration with”, “administering”, “combination”, or “co-therapy”, when referring to use of a Cox-2 inhibitor in combination with a muscarinic receptor antagonist, are intended to embrace administration of each agent in a sequential manner in a regimen that will provide beneficial effects of the drug combination, and is intended as well to embrace co-administration of these agents in a substantially simultaneous manner. Thus, the Cox-2 inhibitor and muscarinic receptor antagonist may be administered in one therapeutic dosage form, such as in a single capsule, tablet, or injection, or in two separate therapeutic dosage forms, such as in separate capsules, tablets, or injections.

Sequential administration of such treatments encompasses both relatively short and relatively long periods between the administration of each of the drugs of the present method. However, for purposes of the present invention, the second drug is administered while the first drug is still having an efficacious effect on the subject. Thus, the present invention takes advantage of the fact that the simultaneous presence of the combination of a Cox-2 inhibitor and muscarinic receptor antagonist in a subject has a greater efficacy than the administration of either agent alone.

Preferably, the second of the two drugs is to be given to the subject within the therapeutic response time of the first drug to be administered. For example, the present invention encompasses administration of a Cox-2 inhibitor to the subject and the later administration of a muscarinic receptor antagonist, as long as the muscarinic receptor antagonist is administered to the subject while the Cox-2 inhibitor is still present in the subject at a level, which in combination with the level of the muscarinic receptor antagonist is therapeutically effective, and vice versa. As used herein, the term “therapeutic response time” means the duration of time that a compound is present or detectable at any level within a subject's body.

In one embodiment, the Cox-2 inhibitor and muscarinic receptor antagonist are administered in the subject in multiple dosage forms. Thus, the Cox-2 inhibitor and muscarinic receptor antagonist are administered in one therapeutic dosage form, such as in a single capsule, tablet, or injection, or in two separate therapeutic dosage forms, such as in separate capsules, tablets, or injections.

The present invention encompasses a novel method of preventing or treating respiratory disorders and respiratory disorder-related complications in a subject comprising administering to the subject at least one Cox-2 inhibitor and one or more muscarinic receptor antagonists.

In one another embodiment, the present invention provides a method for preventing respiratory disorders and respiratory disorder-related complications in a subject comprising administering to the subject a Cox-2 inhibitor in combination with a muscarinic receptor antagonist.

As used herein, the terms “to prevent”, “preventing”, or “prevention” refer to any reduction, no matter how slight, of a subject's predisposition or risk for developing a respiratory disorder or respiratory disorder-related complication. This definition includes either preventing the onset of a respiratory disorder or respiratory disorder-related complication altogether or preventing the onset of a preclinically evident stage of a respiratory disorder or respiratory disorder-related complication in individuals at risk.

In yet another embodiment, the present invention provides a method for treating respiratory disorders or respiratory disorder-related complications in a subject comprising administering to the subject a Cox-2 inhibitor in combination with a muscarinic receptor antagonist.

As used herein, the terms “treating”, “treatment”, “treated”, or “to treat,” mean to alleviate symptoms, eliminate the causation either on a temporary or permanent basis, or to alter or slow the appearance of symptoms or symptom worsening. The term “treatment” includes alleviation or elimination of causation of the symptoms associated with, but not limited to, any of the respiratory disorders or respiratory disorder-related complications described herein. Thus, the combination therapy embodiment of the present invention also provides for the treatment of respiratory disorder-related symptoms, which may arise indirectly from having a respiratory disorder, by treating the underlying respiratory disorder itself.

Without being bound by this or any other theory, it is believed that a therapy comprising a Cox-2 inhibitor and a muscarinic receptor antagonist is efficacious for preventing or treating respiratory disorders and respiratory disorder-related complications. Moreover, in preferred embodiments, the combination of a Cox-2 inhibitor and a muscarinic receptor antagonist provides synergistic effects, which reduce the symptoms associated with respiratory disorders and respiratory disorder-related complications to a greater extent than would be expected based on the administration of either one alone. The term “synergistic” refers to the combination of a Cox-2 inhibitor and a muscarinic receptor antagonist as a combined therapy having an efficacy for the prevention and treatment of respiratory disorders that is greater than the sum of their individual effects.

The synergistic effects of preferred embodiments of the present invention's combination therapy encompass additional unexpected advantages for the treatment and prevention of respiratory disorders. Such additional advantages include, but are not limited to, lowering the required dose of muscarinic receptor antagonists, reducing the side-effects of muscarinic receptor antagonists, and rendering those antagonists more tolerable to subjects in need of respiratory disorder therapy.

The combination therapy of the present invention also provides for the treatment of respiratory disorder-related complications, which may arise indirectly from having a respiratory disorder, by treating the underlying respiratory disorder itself. For example, if a subject is suffering from a respiratory disorder-related complication, such as respiratory failure, the treatment of the underlying respiratory disorder, such as COPD, by the methods and compositions of the present invention will likewise improve the symptoms of the associated complication.

One component of the present invention is a Cox-2 inhibitor.

Inhibitors of the Cox pathway in the metabolism of arachidonic acid that are used in the treatment, prevention or reduction of respiratory disorders and respiratory disorder-related complications, may inhibit enzyme activity through a variety of mechanisms. By way of example, the Cox-2 inhibitors used in the methods described herein may block the enzyme activity directly by binding at the substrate site of the enzyme. In preferred embodiments, the use of a Cox-2 selective inhibitor is highly advantageous in that it minimizes the gastric side effects that can occur with non-selective non-steroidal anti-inflammatory drugs (NSAIDs), especially where prolonged treatment is expected.

The terms “cyclooxygenase-2 inhibitor”, or “Cox-2 inhibitor”, which can be used interchangeably herein, embrace compounds, which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, mixed isomer, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, a 4-(nitrooxy)butyl ester, and mixtures thereof.

Further preferred NSAID compounds include ibuprofen, naproxen, sulindac, ketoporfen, fenoprofen, tiaprofenic acid, suprofen, etodolac, carprofen, ketrolac, piprofen, indoprofen, salicylic acid, flurbiprofen, and mixtures thereof.

In a preferred embodiment, the Cox-2 inhibitor is a Cox-2 selective inhibitor. The term “Cox-2 selective inhibitor” embraces compounds, which selectively inhibit the Cox-2 enzyme over the Cox-1 enzyme, and also include pharmaceutically acceptable salts and prodrugs of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of Cox activity. Preferred Cox-2 selective inhibitors of the present invention have a Cox-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598.

The Cox-2 selective inhibitor of the present invention can be, for example, the Cox-2 selective inhibitor meloxicam, Formula B-1 (CAS registry number 71125-38-7), or a pharmaceutically acceptable salt or prodrug thereof.

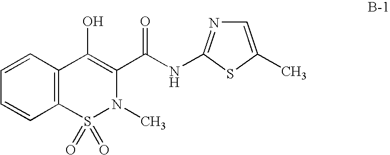

In another embodiment of the invention the Cox-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

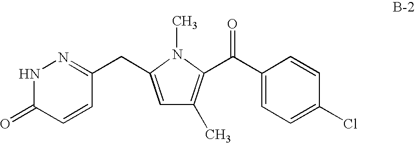

The meaning of any substituent at any one occurrence in Formula I, or any other general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1 yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “oxo” means a single double-bonded oxygen.

The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain.

The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl.

The term “heterocyclyl” means a saturated or unsaturated mono- or multi-ring carbocycle wherein one or more carbon atoms is replaced by N, S, P, or O. This includes, for example, structures such as: 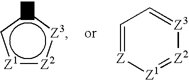
where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures: 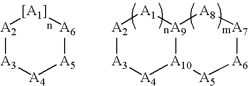
   where:
when n=1, m=1 and A₁-A₈ are each CR^{x} or N, A₉ and A₁₀ are carbon;
when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A₉ and A₁₀ are carbon;
when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;
when n is greater than or equal to 0, and m greater than or equal to 0, atoms separated by 2 atoms (i.e., A₁ and A₄) are Sp3 O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃— (CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂ (—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl.

The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl.

The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N,N-dialkylamino” denote amino groups which have been substituted with one alkyl radical and with two alkyl radicals, respectively.

The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as: 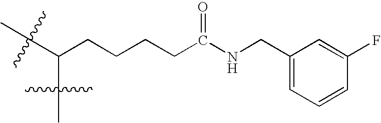
may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if R^{x} is defined as having more than one possible identity.

In one embodiment of the present invention, the Cox-2 selective inhibitor is of the chromene/chroman structural class, which encompasses substituted benzopyrans or substituted benzopyran analogs, as well as substituted benzothiopyrans, dihydroquinolines, or dihydronaphthalenes having the structure of any one of the general Formulas I, II, III, IV, V, and VI, shown below, and including, by way of non-limiting example, the structures disclosed in Table 1, and the diastereomers, enantiomers, racemates, tautomers, salts, esters, amides and prodrugs thereof.

Benzopyrans that can serve as a Cox-2 selective inhibitor of the present invention include substituted benzopyran derivatives that are described in U.S. Pat. Nos. 6,271,253 and 6,492,390. One such class of compounds is defined by the general formula shown below in formula I: 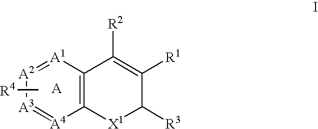
wherein X¹ is selected from O, S, CR^{c}R^{b} and NR^{a};
wherein R^{a} is selected from hydrido, C₁-C₃-alkyl, (optionally substituted phenyl)-C₁-C₃-alkyl, acyl and carboxy-C₁-C₆-alkyl; wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;
wherein R¹ is selected from carboxyl, aminocarbonyl, C₁-C₆-alkylsulfonylaminocarbonyl and C₁-C₆-alkoxycarbonyl;
wherein R² is selected from hydrido, phenyl, thienyl, C₁-C₆-alkyl and C₂-C₆-alkenyl;
wherein R³ is selected from C₁-C₃-perfluoroalkyl, chloro, C₁-C₆-alkylthio, C₁-C₆-alkoxy, nitro, cyano and cyano-C₁-C₃-alkyl;
wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl-C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

Another class of benzopyran derivatives that can serve as the Cox-2 selective inhibitor of the present invention includes compounds having the structure of formula II: 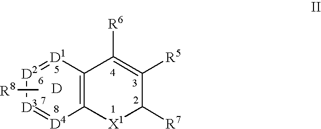
wherein X² is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;
or wherein CR^{c}R^{b} form a cyclopropyl ring;
wherein R⁵ is selected from carboxyl, aminocarbonyl, C₁-C₆-alkylsulfonylaminocarbonyl and C₁-C₆-alkoxycarbonyl;
wherein R⁶ is selected from hydrido, phenyl, thienyl, C₂-C₆-alkynyl and C₂-C₆-alkenyl;
wherein R⁷ is selected from C₁-C₃-perfluoroalkyl, chloro, C₁-C₆-alkylthio, C₁-C₆-alkoxy, nitro, cyano and cyano-C₁-C₃-alkyl;
wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂ O, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the D ring atoms D¹, D², D³ and D⁴ are independently selected from carbon and nitrogen with the proviso that at least two of D¹, D², D³ and D⁴ are carbon; or
wherein R⁸ together with ring D forms a radical selected from naphthyl, quinolyl, isoquinolyl, quinolizinyl, quinoxalinyl and dibenzofuryl; or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III: 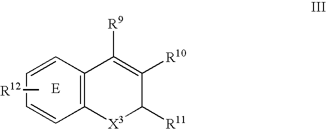
wherein X³ is selected from the group consisting of O or S or NR^{a};
wherein R^{a} is alkyl;
wherein R⁹ is selected from the group consisting of H and aryl;
wherein R¹⁰ is selected from the group consisting of carboxyl, aminocarbonyl, alkylsulfonylaminocarbonyl and alkoxycarbonyl;
wherein R¹¹ is selected from the group consisting of haloalkyl, alkyl, aralkyl, cycloalkyl and aryl optionally substituted with one or more radicals selected from alkylthio, nitro and alkylsulfonyl; and
wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or
wherein R¹² together with ring E forms a naphthyl radical; or an isomer or pharmaceutically acceptable salt thereof; and including the diastereomers, enantiomers, racemates, tautomers, salts, esters, amides and prodrugs thereof.

A related class of compounds useful as Cox-2 selective inhibitors in the present invention is described by Formulas IV and V below: 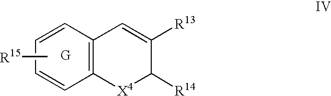
wherein X⁴ is selected from O or S or NR^{a};
wherein R^{a} is alkyl;
wherein R¹³ is selected from carboxyl, aminocarbonyl, alkylsulfonylaminocarbonyl and alkoxycarbonyl;
wherein R¹⁴ is selected from haloalkyl, alkyl, aralkyl, cycloalkyl and aryl optionally substituted with one or more radicals selected from alkylthio, nitro and alkylsulfonyl; and
wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R¹⁵ together with ring G forms a naphthyl radical; or an isomer or pharmaceutically acceptable salt thereof.

Formula V is: 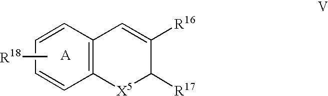
wherein:
X⁵ is selected from the group consisting of O or S or NR^{b}; R^{b} is alkyl;
R¹⁶ is selected from the group consisting of carboxyl, aminocarbonyl, alkylsulfonylaminocarbonyl and alkoxycarbonyl;
R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical; or an isomer or pharmaceutically acceptable salt thereof.

The Cox-2 selective inhibitor may also be a compound of Formula V, wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of lower haloalkyl, lower cycloalkyl and phenyl; and
R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered nitrogen-containing heterocyclosulfonyl, lower alkylsulfonyl, optionally substituted phenyl, lower aralkylcarbonyl, and lower alkylcarbonyl; or wherein R¹⁸ together with ring A forms a naphthyl radical; or an isomer or pharmaceutically acceptable salt thereof.

The Cox-2 selective inhibitor may also be a compound of Formula V, wherein:
X⁵ is selected from the group consisting of oxygen and sulfur;
R¹⁶ is carboxyl;
R¹⁷ is lower haloalkyl; and
R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;
   or an isomer or pharmaceutically acceptable salt thereof.

The Cox-2 selective inhibitor may also be a compound of Formula V, wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and
R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;
   or an isomer or pharmaceutically acceptable salt thereof.

The Cox-2 selective inhibitor may also be a compound of Formula V, wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting trifluoromethyl and pentafluoroethyl; and
R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;
   or an isomer or prodrug thereof.

The Cox-2 selective inhibitor of the present invention can also be a compound having the structure of Formula VI: 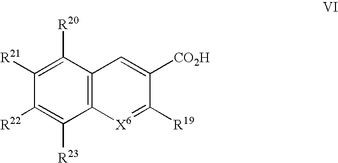
wherein:
X⁶ is selected from the group consisting of O and S;
R¹⁹ is lower haloalkyl;
R²⁰ is selected from the group consisting of hydrido, and halo;
R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;
R²² is selected from the group consisting of hydrido, lower alkyl, halo, lower alkoxy, and aryl; and
R²³ is selected from the group consisting of the group consisting of hydrido, halo, lower alkyl, lower alkoxy, and aryl; or an isomer or prodrug thereof.

The Cox-2 selective inhibitor can also be a compound of having the structure of Formula VI, wherein:
X⁶ is selected from the group consisting of O and S;
R¹⁹ is selected from the group consisting of trifluoromethyl and pentafluoroethyl;
R²⁰ is selected from the group consisting of hydrido, chloro, and fluoro;
R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;
R²² is selected from the group consisting of hydrido, methyl, ethyl, isopropyl, tert-butyl, chloro, methoxy, diethylamino, and phenyl; and
R²³ is selected from the group consisting of hydrido, chloro, bromo, fluoro, methyl, ethyl, tert-butyl, methoxy, and phenyl;

or an isomer or prodrug thereof.

TABLE 1
Examples of Chromene Cox-2 Selective Inhibitors

| Compound | |
|---|---|
| Number | Structural Formula |
| B-3 | 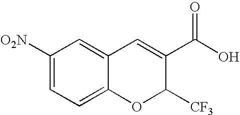 |
| | 6-Nitro-2-trifluoromethyl-2H-1-benzopyran- |
| | 3-carboxylic acid |
| B-4 | 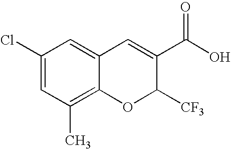 |
| | 6-Chloro-8-methyl-2-trifluoromethyi-2H-1- |
| | benzopyran-3-carboxylic acid |
| B-5 | 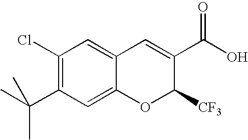 |
| | ((S)-6-Chloro-7-(1,1-dimethylethyl)-2- |
| | (trifluoromethyl-2H-1- |
| | benzopyran-3-carboxylic acid |
| B-6 | 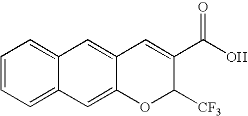 |
| | 2-Trifluoromethyl-2H-naphtho[2,3-b]pyran-3- |
| | carboxylic acid |
| B-7 | 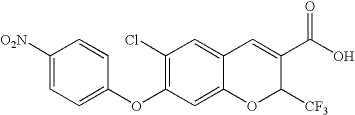 |
| | 6-Chloro-7-(4-nitrophenoxy)-2-(trifluoromethyl)-2H-1- |
| | benzopyran-3-carboxylic acid |
| B-8 | 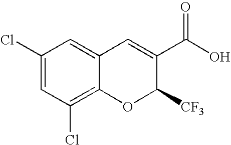 |
| | ((S)-6,8-Dichloro-2-(trifluoromethyl)-2H-1- |
| | benzopyran-3-carboxylic acid |
| B-9 | 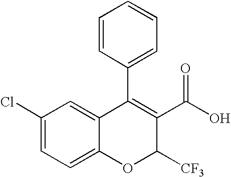 |
| | 6-Chloro-2-(trifluoromethyl)-4-phenyl-2H-1- |
| | benzopyran-3-carboxylic acid |
| B-10 | 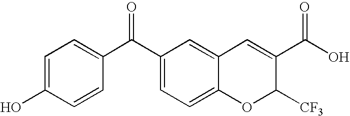 |
| | 6-(4-Hydroxybenzoyl)-2-(trifluoromethyl)-2H-1- |
| | benzopyran-3-carboxylic acid |
| B-11 | 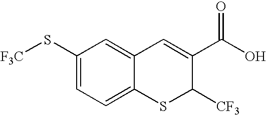 |
| | 2-(Trifluoromethyl)-6-[(trifluoromethyl)thio]-2H-1- |
| | benzothiopyran-3-carboxylic acid |
| B-12 | 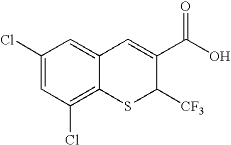 |
| | 6,8-Dichloro-2-trifluoromethyl-2H-1-benzothiopyran- |
| | 3-carboxylic acid |
| B-13 | 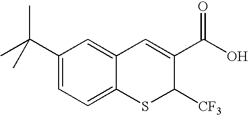 |
| | 6-(1,1-Dimethylethyl)-2-(trifluoromethyl)-2H-1- |
| | benzothiopyran-3-carboxylic acid |
| B-14 | 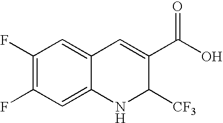 |
| | 6,7-Difluoro-1,2-dihydro-2-(trifluoromethyl)-3- |
| | quinolinecarboxylic acid |
| B-15 | 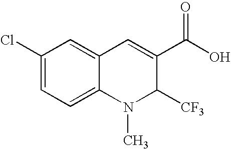 |
| | 6-Chloro-1,2-dihydro-1-methyl-2-(trifluoromethyl)-3- |
| | quinolinecarboxylic acid |
| B-16 | 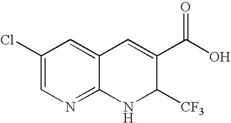 |
| | 6-Chloro-2-(trifluoromethyl)-1,2- |
| | dihydro[1,8]naphthyridine- |
| | 3-carboxylic acid |
| B-17 | 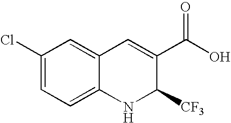 |
| | ((S)-6-Chloro-1,2-dihydro-2-(trifluoromethyl)-3- |
| | quinolinecarboxylic acid |
| B-18 | 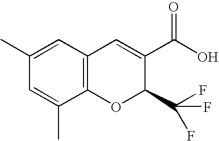 |
| | (2S)-6,8-dimethyl-2-(trifluoromethyl)-2H-chromene- |
| | 3-carboxylic acid |
| B-19 | 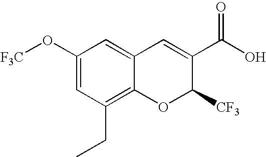 |
| | (2S)-8-ethyl-6-(trifluoromethoxy)-2-(trifluoromethyl)- |
| | 2H-chromene-3-carboxylic acid |
| B-20 | 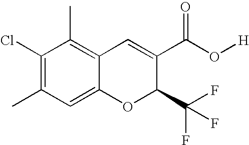 |
| | (2S)-6-chloro-5,7-dimethyl-2-(trifluoromethyl)-2H- |
| | chromene-3-carboxylic acid |

In preferred embodiments, the chromene Cox-2 inhibitor is comprises at least one compound selected from the group consisting of
6-chloro-2-trifluoromethyl-2H-1-benzopyran-3-carboxylic acid,
6-chloro-7-methyl-2-trifluoromethyl-2H-1-benzopyran-3-carboxylic acid,
8-(1-methylethyl)-2-trifluoromethyl-2H-1-benzopyran-3-carboxylic acid,
6-chloro-7-(1,1-dimethylethyl)-2-trifluoromethyl-2H-1-benzopyran-3-carboxylic acid,
6-chloro-8-(1-methylethyl)-2-trifluoromethyl-2H-1-benzopyran-3-carboxylic acid,
2-trifluoromethyl-3H-naphthopyran-3-carboxylic acid,
7-(1,1-dimethylethyl)-2-trifluoromethyl-2H-1-benzopyran-3-carboxylic acid,
6-bromo-2-trifluoromethyl-2H-1-benzopyran-3-carboxylic acid,
8-chloro-2-trifluoromethyl-2H-1-benzopyran-3-carboxylic acid,
6-trifluoromethoxy-2-trifluoromethyl-2H-1-benzopyran-3-carboxylic acid,
5,7-dichloro-2-trifluoromethyl-2H-1-benzopyran-3-carboxylic acid,
8-phenyl-2-trifluoromethyl-2H-1-benzopyran-3-carboxylic acid,
7,8-dimethyl-2-trifluoromethyl-2H-1-benzopyran-3-carboxylic acid,
6,8-bis(dimethylethyl)-2-trifluoromethyl-2H-1-benzopyran-3-carboxylic acid,
7-(1-methylethyl)-2-trifluoromethyl-2H-1-benzopyran-3-carboxylic acid,
7-phenyl-2-trifluoromethyl-2H-1-benzopyran-3-carboxylic acid,
6-chloro-7-ethyl-2-trifluoromethyl-2H-1-benzopyran-3-carboxylic acid,
6-chloro-8-ethyl-2-trifluoromethyl-2H-1-benzopyran-3-carboxylic acid,
6-chloro-7-phenyl-2-trifluoromethyl-2H-1-benzopyran-3-carboxylic acid,
6,7-dichloro-2-trifluoromethyl-2H-1-benzopyran-3-carboxylic acid,
6,8-dichloro-2-trifluoromethyl-2H-1-benzopyran-3-carboxylic acid,
2-trifluoromethyl-3H-naptho[2,1-b]pyran-3-carboxylic acid,
6-chloro-8-methyl-2-trifluoromethyl-2H-1-benzopyran-3-carboxylic acid,
8-chloro-6-methyl-2-trifluoromethyl-2H-1-benzopyran-3-carboxylic acid,
8-chloro-6-methoxy-2-trifluoromethyl-2H-1-benzopyran-3-carboxylic acid,
6-bromo-8-chloro-2-trifluoromethyl-2H-1-benzopyran-3-carboxylic acid,
8-bromo-6-fluoro-2-trifluoromethyl-2H-1-benzopyran-3-carboxylic acid,
8-bromo-6-methyl-2-trifluoromethyl-2H-1-benzopyran-3-carboxylic acid,
8-bromo-5-fluoro-2-trifluoromethyl-2H-1-benzopyran-3-carboxylic acid,
6-chloro-8-fluoro-2-trifluoromethyl-2H-1-benzopyran-3-carboxylic acid,
6-bromo-8-methoxy-2-trifluoromethyl-2H-1-benzopyran-3-carboxylic acid,
6-[[(phenylmethyl)amino]sulfonyl]-2-trifluoromethyl-2H-1-benzopyran-3-carboxylic acid,
6-[(dimethylamino)sulfonyl]-2-trifluoromethyl-2H-1-benzopyran-3-carboxylic acid,
6-[(methylamino)sulfonyl]-2-trifluoromethyl-2H-1-benzopyran-3-carboxylic acid,
6-[(4-morpholino)sulfonyl]-2-trifluoromethyl-2H-1-benzopyran-3-carboxylic acid,
6-[(1,1-dimethylethyl)aminosulfonyl]-2-trifluoromethyl-2H-1-benzopyran-3-carboxylic acid,
6-[(2-methylpropyl)aminosulfonyl]-2-trifluoromethyl-2H-1-benzopyran-3-carboxylic acid,
6-methylsulfonyl-2-trifluoromethyl-2H-1-benzopyran-3-carboxylic acid,
8-chloro-6-[[(phenylmethyl)amino]sulfonyl]-2-trifluoromethyl-2H-1-benzopyran-3-carboxylic acid,
6-phenylacetyl-2-trifluoromethyl-2H-1-benzopyran-3-carboxylic acid,
6,8-dibromo-2-trifluoromethyl-2H-1-benzopyran-3-carboxylic acid,
8-chloro-5,6-dimethyl-2-trifluoromethyl-2H-1-benzopyran-3-carboxylic acid,
6,8-dichloro-(S)-2-trifluoromethyl-2H-1-benzopyran-3-carboxylic acid,
6-benzylsulfonyl-2-trifluoromethyl-2H-1-benzopyran-3-carboxylic acid,
6-[[N-(2-furylmethyl)amino]sulfonyl]-2-trifluoromethyl-2H-1-benzopyran-3-carboxylic acid,
6-[[N-(2-phenylethyl)amino]sulfonyl]-2-trifluoromethyl-2H-1-benzopyran-3-carboxylic acid,
6-iodo-2-trifluoromethyl-2H-1-benzopyran-3-carboxylic acid,
7-(1,1-dimethylethyl)-2-pentafluoroethyl-2H-1-benzopyran-3-carboxylic acid,
6-chloro-2-trifluoromethyl-2H-1-benzothiopyran-3-carboxylic acid.
6-chloro-2-trifluoromethyl-2H-1-benzopyran-3-carboxylic acid,
(S)-6-chloro-2-trifluoromethyl-2H-1-benzopyran-3-carboxylic acid,
6-chloro-7-(1,1-dimethylethyl)-2-trifluoromethyl-2H-1-benzopyran-3-carboxylic acid,
(S)-6-chloro-7-(1,1-dimethylethyl)-2-(trifluoromethyl)-2H-1-benzopyran-3-carboxylic acid,
6-trifluoromethoxy-2-trifluoromethyl-2H-1-benzopyran-3-carboxylic acid,
(S)-6-trifluoromethoxy-2-trifluoromethyl-2H-1-benzopyran-3-carboxylic acid,
6-formyl-2-(trifluoromethyl)-2H-1-benzopyran-3-carboxylic acid,
6-(difluoromethyl)-2-(trifluoromethyl)-2H-1-benzopyran-3-carboxylic acid,
6,8-dichloro-7-methyl-2-(trifluoromethyl)-2H-1-benzopyran-3-carboxylic acid,
6,8-dichloro-2-trifluoromethyl-2H-1-benzopyran-3-carboxylic acid,
(S)-6,8-dichloro-2-(trifluoromethyl)-2H-1-benzopyran-3-carboxylic acid,
6-chloro-1,2-dihydro-2-(trifluoromethyl)-3-quinolinecarboxylic acid,
(S)-6-chloro-1,2-dihydro-2-(trifluoromethyl)-3-quinolinecarboxylic acid,
6,8-dichloro-1,2-dihydro-2-(trifluoromethyl)-3-quinolinecarboxylic acid,
7-(1,1-dimethylethyl)-2-trifluoromethyl-2H-1-benzopyran-3-carboxylic acid,
6,7-dichloro-2-trifluoromethyl-2H-1-benzopyran-3-carboxylic acid,
5,6-dichloro-2-(trifluoromethyl)-2H-1-benzopyran-3-carboxylic acid,
2,6-bis(trifluoromethyl)-2H-1-benzopyran-3-carboxylic acid,
5,6,7-trichloro-2-(trifluoromethyl)-2H-1-benzopyran-3-carboxylic acid,
6,7,8-trichloro-2-(trifluoromethyl)-2H-1-benzopyran-3-carboxylic acid,
6-iodo-1,2-dihydro-2-(trifluoromethyl)-3-quinolinecarboxylic acid,
6-bromo-1,2-dihydro-2-(trifluoromethyl)-3-quinolinecarboxylic acid,
6-chloro-7-methyl-2-(trifluoromethyl)-2H-1-benzothiopyran-3-carboxylic acid,
6,8-dichloro-2-trifluoromethyl-2H-1-benzothiopyran-3-carboxylic acid, and mixtures thereof.

In further preferred embodiments, the chromene Cox-2 inhibitor is selected from (S)-6-chloro-7-(1,1-dimethylethyl)-2-(trifluoromethyl)-2H-1-benzopyran-3-carboxylic acid, (2S)-6,8-dimethyl-2-(trifluoromethyl)-2H-chromene-3-carboxylic acid, (2S)-6-chloro-8-methyl-2-(trifluoromethyl)-2H-chromene-3-carboxylic acid, (2S)-8-ethyl-6-(trifluoromethoxy)-2-(trifluoromethyl)-2H-chromene-3-carboxylic acid, (S)-6,8-dichloro-2-(trifluoromethyl)-2H-1-benzopyran-3-carboxylic acid, (2S)-6-chloro-5,7-dimethyl-2-(trifluoromethyl)-2H-chromene-3-carboxylic acid, and mixtures thereof.

In a preferred embodiment of the invention, the Cox-2 inhibitor can be selected from the class of tricyclic Cox-2 selective inhibitors represented by the general structure of formula VII: 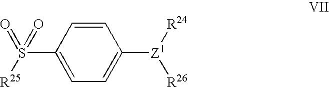
wherein:
Z¹ is selected from the group consisting of partially unsaturated or unsaturated heterocyclyl and partially unsaturated or unsaturated carbocyclic rings;
R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl or amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a prodrug thereof.

In a preferred embodiment of the invention, the tricyclic Cox-2 selective inhibitor comprises at least one compound selected from the group consisting of celecoxib, parecoxib, deracoxib, valdecoxib, lumiracoxib, etoricoxib, rofecoxib, prodrugs of any of them, and mixtures thereof.

In a further preferred embodiment of the invention, the Cox-2 selective inhibitor represented by the above Formula VII is selected from the group of compounds, illustrated in Table 2, which includes celecoxib (B-21), valdecoxib (B-22), deracoxib (B-23), rofecoxib (B-24), etoricoxib (MK-663; B-25), JTE-522 (B-26), or prodrugs thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic Cox-2 Selective Inhibitors

| Compound | |
|---|---|
| Number | Structural Formula |
| B-21 | 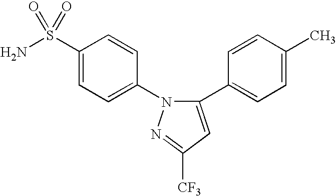 |
| B-22 | 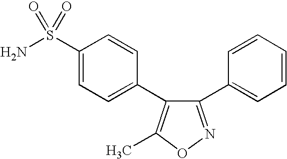 |
| B-23 | 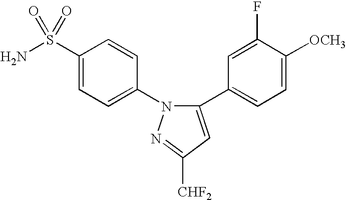 |
| B-24 | 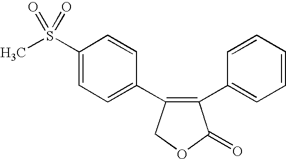 |
| B-25 | 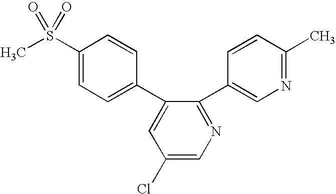 |
| B-26 | 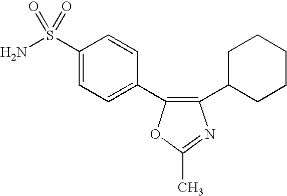 |

In a more preferred embodiment of the invention, the Cox-2 selective inhibitor is selected from the group consisting of celecoxib, rofecoxib and etoricoxib.

In a preferred embodiment, parecoxib (See, U.S. Pat. No. 5,932,598), having the structure shown in B-27, and which is a therapeutically effective prodrug of the tricyclic Cox-2 selective inhibitor valdecoxib, B-22, (See, U.S. Pat. No. 5,633,272), may be advantageously employed as the Cox-2 inhibitor of the present invention.

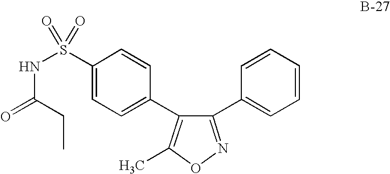

A preferred form of parecoxib is sodium parecoxib.

Another tricyclic Cox-2 selective inhibitor useful in the present invention is the compound ABT-963, having the formula B-28 shown below, that has been previously described in International Publication Number WO 00/24719.

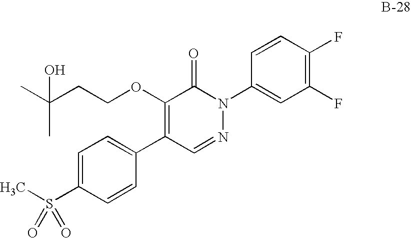

In a further embodiment of the invention, the Cox-2 inhibitor can be selected from the class of phenylacetic acid derivative Cox-2 selective inhibitors represented by the general structure of formula VIII: 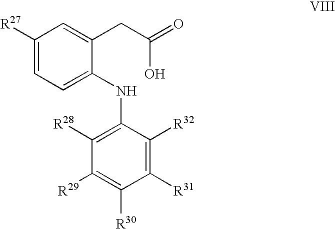
wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxyl;
R³¹ is hydrogen, fluoro, or methyl; and
R³² is chloro, fluoro, trifluoromethyl, methyl, or ethyl, provided that R²⁸, R²⁹, R³⁰ and R³¹ are not all fluoro when R²⁷ is ethyl and R³⁰ is H.

An exemplary phenylacetic acid derivative Cox-2 selective inhibitor that is described in WO 99/11605 is a compound that has the structure shown in formula VIII, wherein:
R²⁷ is ethyl;
R²⁸ and R³⁰ are chloro;
R²⁹ and R³¹ are hydrogen; and
R³² is methyl.

Another phenylacetic acid derivative Cox-2 selective inhibitor is a compound that has the structure shown in formula VIII, wherein:
R²⁷ is propyl;
R²⁸ and R³⁰ are chloro;
R²⁹ and R³¹ are methyl; and
R³² is ethyl.

Another phenylacetic acid derivative Cox-2 selective inhibitor that is disclosed in WO 02/20090 is a compound that is referred to as COX-189 (also termed lumiracoxib; CAS Reg. No. 220991-20-8), having the structure shown in formula VIII, wherein:
R²⁷ is methyl;
R²⁸ is fluoro;
R³² is chloro; and
R²⁹, R³⁰, and R³¹ are hydrogen.

Compounds having a structure similar to that shown in formula VIII, that can serve as the Cox-2 selective inhibitor of the present invention, are described in U.S. Pat. Nos. 6,451,858, 6,310,099, 6,291,523, and 5,958,978.

Other Cox-2 selective inhibitors that can be used in the present invention have the general structure shown in formula IX, where the J group is a carbocycle or a heterocycle. Preferred embodiments have the structure: 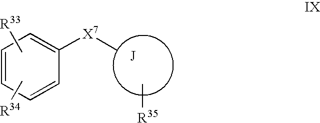
wherein:
X⁷ is O; J is 1-phenyl; R³³ is 2-NHSO₂CH₃; R³⁴ is 4-NO₂; and there is no R³⁵ group, (nimesulide), or
X⁷ is O; J is 1-oxo-inden-5-yl; R³³ is 2-F; R³⁴ is 4-F; and R³⁵ is 6-NHSO₂CH₃, (flosulide); or
X⁷ is O; J is cyclohexyl; R³³ is 2-NHSO₂CH₃; R³⁴ is 5-NO₂; and there is no R³⁵ group, (NS-398); or
X⁷ is S; J is 1-oxo-inden-5-yl; R³³ is 2-F; R³⁴ is 4-F; and R³⁵ is 6-N⁻SO₂CH₃ Na⁺, (L-745337); or
X⁷ is S; J is thiophen-2-yl; R³³ is 4-F; there is no R³⁴ group; and R³⁵ is 5-NHSO₂CH₃, (RWJ-63556); or
X⁷ is O; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

The Cox-2 selective inhibitor NS-398, also known as N-(2-cyclohexyloxynitrophenyl) methane sulfonamide (CAS RN 123653-11-2), having a structure as shown below in formula B-29, has been described in, for example, Yoshimi, N. et al., in Japanese J. Cancer Res., 90(4):406-412 (1999).

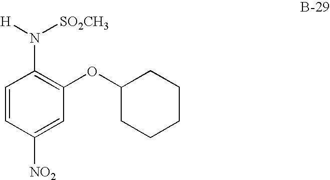

An evaluation of the anti-inflammatory activity of the Cox-2 selective inhibitor, RWJ 63556, in a canine model of inflammation, was described by Kirchner et al., in J Pharmacol Exp Ther 282, 1094-1101 (1997).

Materials that can serve as the Cox-2 selective inhibitor of the present invention include diarylmethylidenefuran derivatives that are described in U.S. Pat. No. 6,180,651. Such diarylmethylidenefuran derivatives have the general formula shown below in formula X: 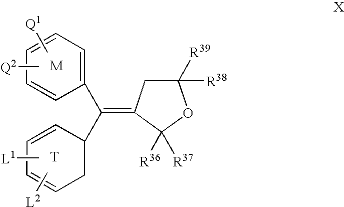
wherein:
the rings T and M independently are a phenyl radical, a naphthyl radical, a radical derived from a heterocycle comprising 5 to 6 members and possessing from 1 to 4 heteroatoms, or a radical derived from a saturated hydrocarbon ring having from 3 to 7 carbon atoms; at least one of the substituents Q¹, Q², L¹ or L² is an —S(O)ₙ—R group, in which n is an integer equal to 0, 1 or 2 and R is a lower alkyl radical having 1 to 6 carbon atoms, a lower haloalkyl radical having 1 to 6 carbon atoms, or an —SO₂NH₂ group;
   and is located in the para position, the others independently being a hydrogen atom, a halogen atom, a lower alkyl radical having 1 to 6 carbon atoms, a trifluoromethyl radical, or a lower O-alkyl radical having 1 to 6 carbon atoms, or Q¹ and Q² or L¹ and L² are a methylenedioxy group; and
R³⁶, R³⁷, R³⁸ and R³⁹ independently are a hydrogen atom, a halogen atom, a lower alkyl radical having 1 to 6 carbon atoms, a lower haloalkyl radical having 1 to 6 carbon atoms, or an aromatic radical selected from the group consisting of phenyl, naphthyl, thienyl, furyl and pyridyl; or,
R³⁶, R³⁷ or R³⁸, R³⁹ are an oxygen atom; or
R³⁶, R³⁷ or R³⁸, R³⁹, together with the carbon atom to which they are attached, form a saturated hydrocarbon ring having from 3 to 7 carbon atoms;
   or an isomer or prodrug thereof.

Particular diarylmethylidenefuran derivatives that can serve as the Cox-2 selective inhibitor of the present invention include, for example, N-(2-cyclohexyloxynitrophenyl)methane sulfonamide, and (E)-4-[(4-methylphenyl)(tetrahydro-2-oxo-3-furanylidene) methyl]benzenesulfonamide.

Other Cox-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Compounds that may act as Cox-2 selective inhibitors of the present invention include multibinding compounds containing from 2 to 10 ligands covanlently attached to one or more linkers, as described in U.S. Pat. No. 6,395,724.

Conjugated linoleic, as described in U.S. Pat. No. 6,077,868, is useful as a Cox-2 selective inhibitor in the present invention.

Compounds that can serve as a Cox-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI: 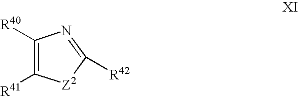
wherein:
Z² is an oxygen atom;
one of R⁴⁰ and R⁴¹ is a group of the formula 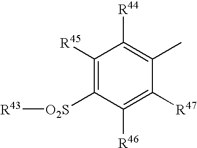
   wherein:
R⁴³ is lower alkyl, amino or lower alkylamino; and
R⁴⁴, R⁴⁵, R⁴⁶ and R⁴⁷ are the same or different and each is hydrogen atom, halogen atom, lower alkyl, lower alkoxy, trifluoromethyl, hydroxyl or amino,
   provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and
R³⁰ is a lower alkyl or a halogenated lower alkyl, and a pharmaceutically acceptable salt thereof.

Cox-2 selective inhibitors that are useful in the method and compositions of the present invention include compounds that are described in U.S. Pat. Nos. 6,080,876 and 6,133,292, and described by formula XII: 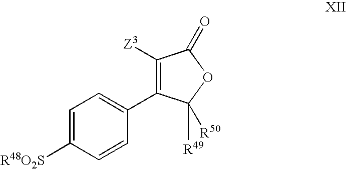
wherein:
Z³ is selected from the group consisting of linear or branched C₁-C₆ alkyl, linear or branched C₁-C₆ alkoxy, unsubstituted, mono-, di- or tri-substituted phenyl or naphthyl wherein the substituents are selected from the group consisting of hydrogen, halo, C₁-C₃ alkoxy, CN, C₁-C₃ fluoroalkyl C₁-C₃ alkyl, and —CO₂H;
R⁴⁸ is selected from the group consisting of NH₂ and CH₃,
R⁴⁹ is selected from the group consisting of C₁-C₆ alkyl unsubstituted or substituted with C₃-C₆ cycloalkyl, and C₃-C₆ cycloalkyl;
R⁵⁰ is selected from the group consisting of:
   C₁-C₆ alkyl unsubstituted or substituted with one, two or three fluoro atoms, and C₃-C₆ cycloalkyl;
   with the proviso that R⁴⁹ and R⁵⁰ are not the same.

Pyridines that are described in U.S. Pat. Nos. 6,596,736, 6,369,275, 6,127,545, 6,130,334, 6,204,387, 6,071,936, 6,001,843 and 6,040,450, and can serve as Cox-2 selective inhibitors of the present invention, have the general formula described by formula XIII: 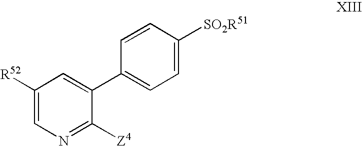
wherein:
R⁵¹ is selected from the group consisting of CH₃, NH₂, NHC(O)CF₃, and NHCH₃;
Z⁴ is a mono-, di-, or trisubstituted phenyl or pyridinyl (or the N-oxide thereof), wherein the substituents are chosen from the group consisting of hydrogen, halo, C₁-C₆ alkoxy, C₁-C₆ alkylthio, CN, C₁-C₆ alkyl, C₁-C₆ fluoroalkyl, N₃, —CO₂R⁵³, hydroxyl, —C(R⁵⁴)(R⁵⁵)—OH, —C₁-C₆ alkyl-CO₂—R⁵⁶, C₁-C₆ fluoroalkoxy;
R⁵² is chosen from the group consisting of: halo, C₁-C₆ alkoxy, C₁-C₆ alkylthio, CN, C₁-C₆ alkyl, C₁-C₆ fluoroalkyl, N₃, —CO₂R⁵⁷, hydroxyl, —C(R⁵⁸)(R⁵⁹)—OH, —C₁-C₆ alkyl-CO₂—R⁶⁰, C₁-C₆ fluoroalkoxy, NO₂, NR⁶¹R⁶², and NHCOR⁶³;
R⁵³, R⁵⁴, R⁵⁵, R⁵⁶, R⁵⁷, R⁵⁸, R⁵⁹, R⁶⁰, R⁶¹, R⁶², and R⁶³, are each independently chosen from the group consisting of hydrogen and C₁-C₆ alkyl;
or R⁵⁴ and R⁵⁵, R⁵⁸ and R⁵⁹, or R⁶¹ and R⁶² together with the atom to which they are attached form a saturated monocyclic ring of 3, 4, 5, 6, or 7 atoms.

Materials that can serve as the Cox-2 selective inhibitor of the present invention include diarylbenzopyran derivatives that are described in U.S. Pat. No. 6,340,694. Such diarylbenzopyran derivatives have the general formula shown below in formula XIV: 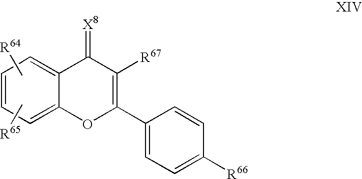
wherein:
X⁸ is an oxygen atom or a sulfur atom;
R⁶⁴ and R⁶⁵, identical to or different from each other, are independently a hydrogen atom, a halogen atom, a C₁-C₆ lower alkyl group, a trifluoromethyl group, an alkoxy group, a hydroxyl group, a nitro group, a nitrile group, or a carboxyl group;
R⁶⁶ is a group of a formula: S(O)NR⁶⁸ wherein n is an integer of 0˜2,
R⁶⁸ is a hydrogen atom, a C₁-C₆ lower alkyl group, or a group of a formula: NR⁶⁹R⁷⁰ wherein R⁶⁹ and R⁷⁰, identical to or different from each other, are independently a hydrogen atom, or a C₁-C₆ lower alkyl group; and
R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures: 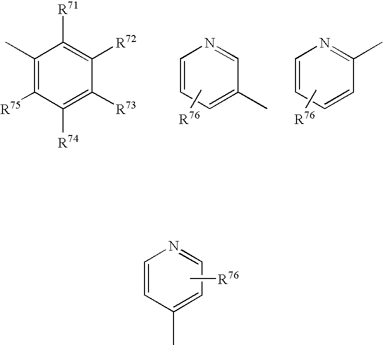
   wherein:
R⁷¹ through R⁷⁵, identical to or different from one another, are independently a hydrogen atom, a halogen atom, a C₁-C₆ lower alkyl group, a trifluoromethyl group, an alkoxy group, a hydroxyl group, a hydroxyalkyl group, a nitro group, a group of a formula: S(O)NR⁶⁸, a group of a formula: NR⁶⁹R⁷⁰, a trifluoromethoxy group, a nitrile group a carboxyl group, an acetyl group, or a formyl group, wherein n, R⁶⁸, R⁶⁹ and R⁷⁰ have the same meaning as defined by R⁶⁶ above; and
R⁷⁶ is a hydrogen atom, a halogen atom, a C₁-C₆ lower alkyl group, a trifluoromethyl group, an alkoxy group, a hydroxyl group, a trifluoromethoxy group, a carboxyl group, or an acetyl group.

Materials that can serve as the Cox-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV: 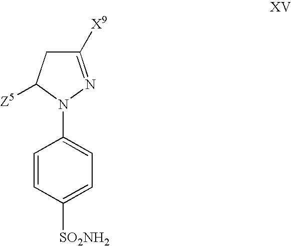
wherein:
X⁹ is selected from the group consisting of C₁-C₆ trihalomethyl, preferably trifluoromethyl; C₁-C₆ alkyl; and an optionally substituted or di-substituted phenyl group of formula XVI: 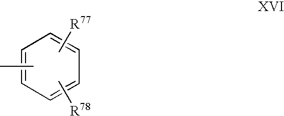
   wherein:
R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;
Z⁵ is selected from the group consisting of substituted and unsubstituted aryl.

Compounds useful as Cox-2 selective inhibitors of the present invention include heterocycles that are described in U.S. Pat. No. 6,153,787. Such heterocycles have the general formulas shown below in formulas XVII and XVIII: 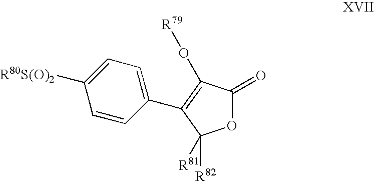
wherein:
R⁷⁹ is a mono-, di-, or tri-substituted C₁-C₁₂ alkyl, or a mono-, or an unsubstituted or mono-, di- or tri-substituted linear or branched C₂-C₁₀ alkenyl, or an unsubstituted or mono-, di- or tri-substituted linear or branched C₂-C₁₀ alkynyl, or an unsubstituted or mono-, di- or tri-substituted C₃-C₁₂ cycloalkenyl, or an unsubstituted or mono-, di- or tri-substituted C₅-C₁₂ cycloalkynyl, wherein the substituents are chosen from the group consisting of halo selected from F, C₁, Br, and 1, OH, CF₃, C₃-C₆ cycloalkyl, ═O, dioxolane, CN;
R⁸⁰ is selected from the group consisting of CH₃, NH₂, NHC(O)CF₃, and NHCH₃;
R⁸¹ and R⁸² are independently chosen from the group consisting of hydrogen and C₁-C₁₀ alkyl;
or R⁸¹ and R⁸² together with the carbon to which they are attached form a saturated monocyclic carbon ring of 3, 4, 5, 6 or 7 atoms.

Formula XVIII is: 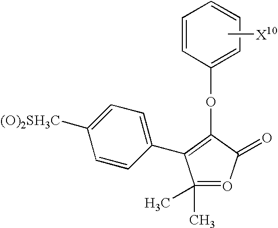
wherein X¹⁰ is fluoro or chloro.

Materials that can serve as the Cox-2 selective inhibitor of the present invention include 2,3,5-trisubstituted pyridines that are described in U.S. Pat. No. 6,046,217. Such pyridines have the general formula shown below in formula XIX: 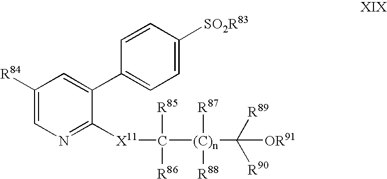
or a pharmaceutically acceptable salt thereof,
wherein:
X¹¹ is selected from the group consisting of O, S, and a bond;
n is 0 or 1;
R⁸³ is selected from the group consisting of CH₃, NH₂, and NHC(O)CF₃;
R⁸⁴ is chosen from the group consisting of halo, C₁-C₆ alkoxy, C₁-C₆ alkylthio, CN, C₁-C₆ alkyl, C₁-C₆ fluoroalkyl, N₃, —CO₂R⁹², hydroxyl, —C(R⁹³)(R⁹⁴)—OH, —C₁-C₆ alkyl-CO₂—R⁹⁵, C₁-C₆ fluoroalkoxy, NO₂, NR⁹⁶R⁹⁷, and NHCOR⁹⁸;
R⁸⁵ to R⁸⁹ are independently chosen from the group consisting of hydrogen and C₁-C₆ alkyl;
or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

Compounds that are useful as the Cox-2 selective inhibitor of the present invention include diaryl bicyclic heterocycles that are described in U.S. Pat. No. 6,329,421. Such diaryl bicyclic heterocycles have the general formula shown below in formula XX: 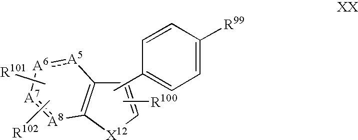
and pharmaceutically acceptable salts thereof wherein:
-A⁵=A⁶-A⁷=A⁸- is selected from the group consisting of:
(a) —CH═CH—CH═CH—,
(b) —CH₂—CH₂—CH₂—C(O)—, —CH₂—CH₂—C(O)—CH₂—, —CH₂—C(O)—CH₂—CH₂, —C(O)—CH₂—CH₂—CH₂,
(c) —CH₂—CH₂—C(O)—, —CH₂—C(O)—CH₂—, —C(O)—CH₂—CH₂—
(d) —CH₂—CH₂—O—C(O)—, CH₂—O—C(O)—CH₂—, —C(O)—CH₂—CH₂—,
(e) —CH₂—CH₂—C(O)—O—, —CH₂—C(O)—OCH₂—C(O)—O—CH₂—CH₂—,
   (f) —C(R¹⁰⁵)₂—O—C(O)—, —C(O)—O—C(R¹⁰⁵)₂—O—C(O)—C(R¹⁰⁵)₂—, —C(R¹⁰⁵)₂—C(O)—O—
(g) —N═CH—CH═CH—,
(h) —CH═N—CH═CH—,
(i) —CH═CH—N═CH—,
(j) —CH═CH—CH═N—,
(k) —N═CH—CH═N—,
(l) —N═CH—N═CH—,
(m) —CH═N—CH═N—,
(n) —S—CH═N—,
(o) —S—N═CH—,
(p) —N═N—NH—,
(q) —CH═N—S—, and
(r) —N═CH—S—;
   R⁹⁹ is selected from the group consisting of S(O)₂CH₃, S(O)₂NH₂, S(O)₂NHCOCF₃, S(O)(NH)CH₃, S(O)(NH)NH₂, S(O)(NH)NHCOCF₃, P(O)(CH₃)OH, and P(O)(CH₃)NH₂;
   R¹⁰⁰ is selected from the group consisting of:
(a) C₁-C₆ alkyl,
(b) C₃-C₇ cycloalkyl,
(c) mono- or di-substituted phenyl or naphthyl wherein the substituent is selected from the group consisting of:
   (1) hydrogen,
   (2) halo, including F, Cl, Br, I,
   (3) C₁-C₆ alkoxy,
   (4) C₁-C₆ alkylthio,
   (5) CN,
   (6) CF₃,
   (7) C₁-C₆ alkyl,
   (8) N₃,
   (9) —CO₂H,
   (10) —CO₂—C₁-C₄ alkyl,
   (11) —C(R¹⁰³)(R¹⁰⁴)—OH,
   (12) —C(R¹⁰³)(R¹⁰⁴)—O—C₁-C₄ alkyl, and
   (13) —C₁-C₆ alkyl-CO₂—R¹⁰⁶;
(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:
   (1) hydrogen,
   (2) halo, including fluoro, chloro, bromo and iodo,
   (3) C₁-C₆ alkyl,
   (4) C₁-C₆ alkoxy,
   (5) C₁-C₆ alkylthio,
   (6) CN,
   (7) CF₃,
   (8) N₃,
   (9) —C(R¹⁰³)(R¹⁰⁴)—OH, and
   (10) —C(R¹⁰³)(R¹⁰⁴)—O—C₁-C₄ alkyl;
(e) benzoheteroaryl which includes the benzo fused analogs of (d);
   R¹⁰¹ and R¹⁰² are the substituents residing on any position of -A⁵=A⁶-A⁷=A⁸—and are selected independently from the group consisting of:
(a) hydrogen,
(b) CF₃,
(c) CN,
(d) C₁-C₆ alkyl,
(e) -Q³ wherein Q³ is Q⁴, CO₂H, C(R¹⁰³)(R¹⁰⁴)OH,
(f) —O-Q⁴,
(g) —S-Q⁴, and
(h) optionally substituted:
   (1) —C₁-C₅ alkyl-Q³,
   (2) —O—C₁-C₅ alkyl-Q³,
   (3) —S—C₁-C₅ alkyl-Q³,
   (4) —C₁-C₃ alkyl-O—C₁₋₃ alkyl-Q³,
   (5) —C₁-C₃ alkyl-S—C₁₋₃ alkyl-Q³,
   (6) —C₁-C₅ alkyl-O-Q⁴,
   (7) —C₁-C₅ alkyl-S-Q⁴,
      wherein the substituent resides on the alkyl chain and the substituent is C₁-C₃ alkyl, and Q³ is Q⁴, CO₂H, C(R¹⁰³)(R¹⁰⁴)OH Q⁴ is CO₂—C₁-C₄ alkyl, tetrazolyl-5-yl, or C(R¹⁰³)(R¹⁰⁴)O—C₁-C₄ alkyl;
   R¹⁰³, R¹⁰⁴ and R¹⁰⁵ are each independently selected from the group consisting of hydrogen and C₁-C₆ alkyl; or
   R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;
   R¹⁰⁶ is hydrogen or C₁-C₆ alkyl;
   R¹⁰⁷ is hydrogen, C₁-C₆ alkyl or aryl;
X⁷ is O, S, NR¹⁰⁷, CO, C(R¹⁰⁷)₂, C(R¹⁰⁷)(OH), —C(R¹⁰⁷)═C(R¹⁰⁷)—; —C(R¹⁰⁷)═N—; or —N═C(R¹⁰⁷)—.

Compounds that may act as Cox-2 selective inhibitors include salts of 5-amino or a substituted amino 1,2,3-triazole compound that are described in U.S. Pat. No. 6,239,137. The salts are of a class of compounds of formula XXI: 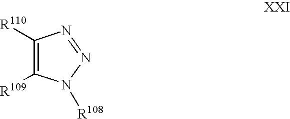
wherein:
R¹⁰⁸ is: 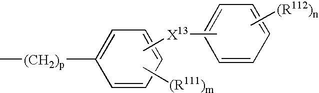
   wherein:
p is 0 to 2; m is 0 to 4; and n is 0 to 5;
X¹³ is O, S, SO, SO₂, CO, CHCN, CH₂ or C═NR¹¹³ where R¹¹³ is hydrogen, loweralkyl, hydroxyl, loweralkoxy, amino, loweralkylamino, diloweralkylamino or cyano;
R¹¹¹ and R¹¹² are independently halogen, cyano, trifluoromethyl, loweralkanoyl, nitro, loweralkyl, loweralkoxy, carboxy, lowercarbalkoxy, trifuloromethoxy, acetamido, loweralkylthio, loweralkylsulfinyl, loweralkylsulfonyl, trichlorovinyl, trifluoromethylthio, trifluoromethylsulfinyl, or trifluoromethylsulfonyl;
R¹⁰⁹ is amino, mono or diloweralkyl amino, acetamido, acetimido, ureido, formamido, or guanidino; and
R¹¹⁰ is carbamoyl, cyano, carbazoyl, amidino or N-hydroxycarbamoyl; wherein the loweralkyl, loweralkyl containing, loweralkoxy and loweralkanoyl groups contain from 1 to 3 carbon atoms.

Pyrazole derivatives such as those described in U.S. Pat. No. 6,136,831 can serve as a Cox-2 selective inhibitor of the present invention. Such pyrazole derivatives have the formula shown below in formula XXII: 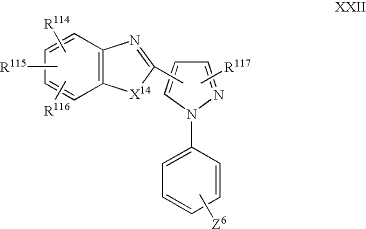
wherein:
R¹¹⁴ is hydrogen or halogen;
R¹¹⁵ and R¹¹⁶ are each independently hydrogen, halogen, lower alkyl, lower alkoxy, hydroxyl or lower alkanoyloxy;
R¹¹⁷ is lower haloalkyl or lower alkyl;
X¹⁴ is sulfur, oxygen or NH; and
Z⁶ is lower alkylthio, lower alkylsulfonyl or sulfamoyl;
   or a pharmaceutically acceptable salt thereof.

Materials that can serve as a Cox-2 selective inhibitor of the present invention include substituted derivatives of benzosulphonamides that are described in U.S. Pat. No. 6,297,282. Such benzosulphonamide derivatives have the formula shown below in formula XXIII: 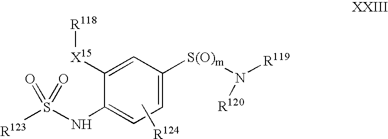
wherein:
X¹⁵ denotes oxygen, sulphur or NH;
R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;
R¹¹⁹ and R¹²⁰, independently from one another, denote hydrogen, an optionally polyfluorised alkyl group, an aralkyl, aryl or heteroaryl group or a group (CH₂)ₙ—X¹⁶; or
R¹¹⁹ and R¹²⁰, together with the N-atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;
X¹⁶ denotes halogen, NO₂, —OR¹²¹, —COR¹²¹, —CO₂R¹²¹, —OCO₂R¹²¹, —CN, —CONR¹²¹ OR¹²², —CONR²¹R¹²², —SR¹²¹, —S(O)R¹²¹, —S(O)₂R¹²¹, —NR¹²¹R¹²², —NHC(O)R¹²¹—NHS(O)₂R¹²¹;
n denotes a whole number from 0 to 6;
R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;
R¹²⁴ denotes halogen, hydroxyl, a straight-chained or branched alkyl, alkoxy, acyloxy or alkyloxycarbonyl group with 1-6 C-atoms, which can optionally be mono- or polysubstituted by halogen, NO₂, —OR¹²¹, —COR¹²¹, —CO₂R¹²¹ C₂R¹²¹, —CN, —CONR¹²¹OR¹²², —CONR¹²¹ R¹²², —SR¹²¹, —S(O)R¹²¹, —S(O)₂R¹², —NR¹²¹R¹²², —NHC(O)R¹²¹, —NHS(O)₂R¹²¹, or a polyfluoroalkyl group;
R¹²¹ and R¹²², independently from one another, denote hydrogen, alkyl, aralkyl or aryl; and
m denotes a whole number from 0 to 2;
   and the pharmaceutically acceptable salts thereof.

Compounds that are useful as Cox-2 selective inhibitors of the present invention include phenyl heterocycles that are described in U.S. Pat. Nos. 5,474,995 and 6,239,173. Such phenyl heterocyclic compounds have the formula shown below in formula XXIV: 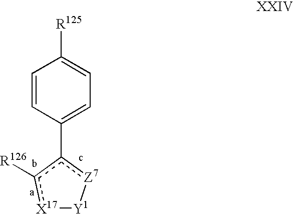
or pharmaceutically acceptable salts thereof wherein:
X¹⁷—Y¹-Z⁷-is selected from the group consisting of:
(a) —CH₂ CH₂ CH₂—,
(b) —C(O)CH₂ CH₂—,
(c) —CH₂ CH₂ C(O)—,
(d) —CR¹²⁹ (R^{129′})—O—C(O)—,
(e) —C(O)—O—CR¹²⁹ (R^{129′})—,
(f) —CH₂—NR¹²⁷—CH₂—,
(g) —CR¹²⁹ (R¹²⁹′)—NR¹²⁷—C(O)
(h) —CR¹²⁸═CR¹²⁸—S—
(i) —S—CR¹²⁸═CR^{128′}—,
(j)—S—N═CH—,
(k) —CH═N—S—,
(l) —N═CR¹²⁸—O—,
(m) —O—CR¹²⁸═N—,
(n) —N═CR¹²⁸—NH—,
(o)—N═CR¹²⁸—S—, and
(p) —S—CR¹²⁸═N—,
(q) —C(O)—NR¹²⁷—CR¹²⁹ (R¹²⁹′)
(r) —R¹²⁷ N—CH═CH— provided R¹²² is not —S(O)₂CH₃,
(s) —CH═CH—NR¹²⁷-provided R¹²⁵ is not —S(O)₂CH₃;
   when side b is a double bond, and sides a and c are single bonds; and X¹⁷—Y¹-Z⁷-is selected from the group consisting of:
(a) ═CH—O—CH═, and
(b) ═CH—NR¹²⁷—CH═,
(c) ═N—S—CH═,
(d) ═CH—S—N═,
(e) ═N—O—CH═,
(f) ═CH—O—N═,
(g) ═N—S—N═,
(h) ═N—O—N═,
   when sides a and c are double bonds and side b is a single bond; R¹²⁵ is selected from the group consisting of:
(a) S(O)₂ CH₃,
(b) S(O)₂ NH₂,
(c) S(O)₂ NHC(O)CF₃,
(d) S(O)(NH)CH₃,
(e) S(O)(NH)NH₂,
(f) S(O)(NH)NHC(O)CF₃,
(g) P(O)(CH₃)OH, and
(h) P(O)(CH₃)NH₂;
   R¹²⁶ is selected from the group consisting of
(a) C₁-C₆ alkyl,
(b) C₃, C₄, C₅, C₆, and C₇, cycloalkyl,
(c) mono-, di- or tri-substituted phenyl or naphthyl, wherein the substituent is selected from the group consisting of:
   (1) hydrogen,
   (2) halo,
   (3) C₁-C₆ alkoxy,
   (4) C₁-C₆ alkylthio,
   (5) CN,
   (6) CF₃,
   (7) C₁-C₆ alkyl,
   (8) N₃,
   (9) —CO₂H,
   (10) —CO₂—C₁-C₄ alkyl,
   (11) —C(R¹²⁹)(R¹³⁰)—OH,
   (12) —C(R¹²⁹)(R¹³⁰)—O—C₁-C₄ alkyl, and
   (13) —C₁-C₆ alkyl-CO₂—R¹²⁹;
(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of:
   (1) hydrogen,
   (2) halo, including fluoro, chloro, bromo and iodo,
   (3) C₁-C₆ alkyl,
   (4) C₁-C₆ alkoxy,
   (5) C₁-C₆ alkylthio,
   (6) CN,
   (7) CF₃,
   (8) N₃,
   (9) —C(R¹²⁹)(R¹³⁰)—OH, and
   (10) —C(R¹²⁹)(R¹³⁰)—O—C₁-C₄ alkyl;
(e) benzoheteroaryl which includes the benzo fused analogs of (d);
   R¹²⁷ is selected from the group consisting of:
(a) hydrogen,
(b) CF₃,
(c) CN,
(d) C₁-C₆ alkyl,
(e) hydroxyl C₁-C₆ alkyl,
(f) —C(O)—C₁-C₆ alkyl,
(g) optionally substituted:
   (1) —C₁-C₅ alkyl-Q⁵,
   (2) —C₁-C₅ alkyl-O—C₁-C₃ alkyl-Q⁵,
   (3) —C₁-C₃ alkyl-S—C₁-C₃ alkyl-Q⁵,
   (4) —C₁-C₅ alkyl-O-Q⁵, or
   (5) —C₁-C₅ alkyl-S-Q⁵,
   wherein the substituent resides on the alkyl and the substituent is C₁-C₃ alkyl;
(h)-Q⁵;
   R¹²⁸ and R^{128′} are each independently selected from the group consisting of:
(a) hydrogen,
(b) CF₃,
(c) CN,
(d) C₁-C₆ alkyl,
(e) -Q⁵,
(f) —O-Q⁵;
(g) —S-Q⁵, and
(h) optionally substituted:
   (1) —C₁-C₅ alkyl-Q⁵,
   (2) —O—C₁-C₅ alkyl-Q⁵,
   (3) —S—C₁-C₅ alkyl-Q⁵,
   (4) —C₁-C₃ alkyl-O—C₁-C₃ alkyl-Q⁵,
   (5) —C₁-C₃ alkyl-S—C₁-C₃ alkyl-Q⁵,
   (6) —C₁-C₅ alkyl-O-Q⁵,
   (7) —C₁-C₅ alkyl-S-Q⁵,
   wherein the substituent resides on the alkyl and the substituent is C₁-C₃ alkyl, and
   R¹²⁹, R¹²⁹′, R¹³⁰, R¹³¹ and R¹³² are each independently selected from the group consisting of:
(a) hydrogen,
(b) C₁-C₆ alkyl;
or R¹²⁹ and R¹³⁰ or R¹³¹ and R¹³² together with the carbon to which they are attached form a saturated monocyclic carbon ring of 3, 4, 5, 6 or 7 atoms;
   Q⁵ is CO₂H, CO₂—C₁-C₄ alkyl, tetrazolyl-5-yl, C(R¹³¹)(R¹³²)(OH), or
      C(R¹³¹)(R¹³²)(O—C₁-C₄ alkyl);
      provided that when X—Y-Z is —S—CR¹²⁸═CR¹²⁸ then R¹²⁸ and R¹²⁸ are other than CF₃.

An exemplary phenyl heterocycle that is disclosed in U.S. Pat. No. 6,239,173 is 3-phenyl-4-(4-(methylsulfonyl)phenyl)-2-(2H)-furanone.

Bicycliccarbonyl indole compounds such as those described in U.S. Pat. No. 6,303,628 are useful as Cox-2 selective inhibitors of the present invention. Such bicycliccarbonyl indole compounds have the formula shown below in formula XXV: 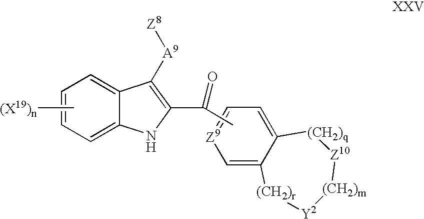
or the pharmaceutically acceptable salts thereof wherein:
A⁹ is C₁-C₆ alkylene or —NR¹³³—;
Z⁸ is C(=L³)R¹³⁴, or SO₂R¹³⁵;
Z⁹ is CH or N;
Z¹⁰ and Y² are independently selected from —CH₂—, S and —N—R¹³³;
m is 1, 2 or 3;
q and r are independently 0, 1 or 2;
X¹⁸ is independently selected from halogen, C₁-C₄ alkyl, halo-substituted C₁-C₄ alkyl, hydroxyl, C₁-C₄ alkoxy, halo-substituted C₁-C₄ alkoxy, C₁-C₄ alkylthio, nitro, amino, mono- or di-(C₁-C₄ alkyl)amino and cyano;
   n is 0, 1, 2, 3 or 4;
L³ is oxygen or sulfur; R¹³³ is hydrogen or C₁-C₄ alkyl;
   R¹³⁴ is hydroxyl, C₁-C₆ alkyl, halo-substituted C₁-C₆ alkyl, C₁-C₆ alkoxy, halo-substituted C₁-C₆ alkoxy, C₃-C₇ cycloalkoxy, C₁-C₄ alkyl(C₃-C₇ cycloalkoxy), —NR¹³⁶R¹³⁷, C₁-C₄ alkylphenyl-O— or phenyl-O—, said phenyl being optionally substituted with one to five substituents independently selected from halogen, C₁-C₄ alkyl, hydroxyl, C₁-C₄ alkoxy and nitro;
   R¹³⁵ is C₁-C₆ alkyl or halo-substituted C₁-C₆ alkyl; and
   R¹³⁶ and R¹³⁷ are independently selected from hydrogen, C₁₋₆ alkyl and halo-substituted C₁-C₆ alkyl.

Materials that can serve as a Cox-2 selective inhibitor of the present invention include benzimidazole compounds that are described in U.S. Pat. No. 6,310,079. Such benzimidazole compounds have the formula shown below in formula XXVI: 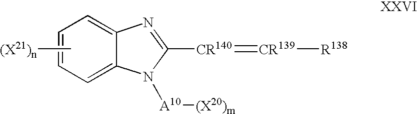
or a pharmaceutically acceptable salt thereof, wherein:
A¹⁰ is heteroaryl selected from
a 5-membered monocyclic aromatic ring having one hetero atom selected from O, S and N and optionally containing one to three N atom(s) in addition to said hetero atom, or
a 6-membered monocyclic aromatic ring having one N atom and optionally containing one to four N atom(s) in addition to said N atom; and said heteroaryl being connected to the nitrogen atom on the benzimidazole through a carbon atom on the heteroaryl ring;
X²⁰ is independently selected from halo, C₁-C₄ alkyl, hydroxyl, C₁-C₄ alkoxy, halo-substituted C₁-C₄ alkyl, hydroxyl-substituted C₁-C₄ alkyl, (C₁-C₄ alkoxy)C₁-C₄ alkyl, halo-substituted C₁-C₄ alkoxy, amino, N—(C₁-C₄ alkyl)amino, N,N-di(C₁-C₄ alkyl)amino, [N—(C₁-C₄ alkyl)amino]C₁-C₄ alkyl, [N,N-di(C₁-C₄ alkyl)amino]C₁-C₄ alkyl, N—(C₁-C₄ alkanoyl)amonio, N—(C₁-C₄ alkyl)(C₁-C₄ alkanoyl)amino, N—[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N—(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N,N-di(C₁-C₄ alkyl)amino]sulfonyl;
X²¹ is independently selected from halo, C₁-C₄ alkyl, hydroxyl, C₁-C₄ alkoxy, halo-substituted C₁-C₄ alkyl, hydroxyl-substituted C₁-C₄ alkyl, (C₁-C₄ alkoxy)C₁-C₄ alkyl, halo-substituted C₁-C₄ alkoxy, amino, N—(C₁-C₄ alkyl)amino, N,N-di(C₁-C₄ alkyl)amino, [N—(C₁-C₄ alkyl)amino]C₁-C₄ alkyl, [N,N-di(C₁-C₄ alkyl)amino]C₁-C₄ alkyl, N—(C₁-C₄ alkanoyl)amino, N—(C₁-C₄ alkyl)-N-(C₁-C₄ alkanoyl) amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)hydroxyl, cabamoyl, [N—(C₁-C₄ alkyl) amino]carbonyl, [N,N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N,N-di(C₁-C₄ alkyl)amino]sulfonyl;
R¹³⁸ is selected from:
hydrogen;
straight or branched C₁-C₄ alkyl optionally substituted with one to three substituent(s) wherein said substituents are independently selected from halo, hydroxyl, C₁-C₄ alkoxy, amino, N—(C₁-C₄ alkyl)amino and N, N-di(C₁-C₄ alkyl)amino;
C₃-C₈ cycloalkyl optionally substituted with one to three substituent(s) wherein said substituents are independently selected from halo, C₁-C₄ alkyl, hydroxyl, C₁-C₄ alkoxy, amino, N—(C₁-C₄ alkyl)amino and N, N-di(C₁-C₄ alkyl)amino; C₄-C₈ cycloalkenyl optionally substituted with one to three substituent(s) wherein said substituents are independently selected from halo, C₁-C₄ alkyl, hydroxyl, C₁-C₄ alkoxy, amino, N—(C₁-C₄ alkyl)amino and N,N-di(C₁-C₄ alkyl)amino;
phenyl optionally substituted with one to three substituent(s) wherein said substituents are independently selected from halo, C₁-C₄ alkyl, hydroxyl, C₁-C₄ alkoxy, halo-substituted C₁-C₄ alkyl, □ydroxyl -substituted C₁-C₄ alkyl, (C₁-C₄ alkoxy)C₁-C₄ alkyl, halo-substituted C₁-C₄ alkoxy, amino, N—(C₁-C₄ alkyl)amino, N,N-di(C₁-C₄ alkyl)amino, [N—(C₁-C₄ alkyl)amino]C₁-C₄ alkyl, [N,N-di(C₁-C₄ alkyl)amino]C₁-C₄ alkyl, N—(C₁-C₄ alkanoyl)amino, N—[C₁-C₄ alkyl)(C₁-C₄ alkanoyl)]amino, N—[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbomoyl, [N—(C₁-C₄ alky)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N,N-di(C₁-C₄ alkyl)amino]sulfonyl; and
heteroaryl selected from:
a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and
said heteroaryl being optionally substituted with one to three substituent(s) selected from X²⁰;
R¹³⁹ and R¹⁴⁰ are independently selected from:
hydrogen;
halo;
C₁-C₄ alkyl;
phenyl optionally substituted with one to three substituent(s) wherein said substituents are independently selected from halo, C₁-C₄ alkyl, hydroxyl, C₁-C₄ alkoxy, amino, N—(C₁-C₄ alkyl)amino and N,N-di(C₁-C₄ alkyl)amino;
or R¹³⁸ and R¹³⁹ can form, together with the carbon atom to which they are attached, a C₃-C₇ cycloalkyl ring;
m is 0, 1, 2, 3, 4 or 5; and
n is 0, 1, 2, 3 or 4.

Compounds that may be employed as a Cox-2 selective inhibitor of the present invention include indole compounds that are described in U.S. Pat. No. 6,300,363. Such indole compounds have the formula shown below in formula XXVII: 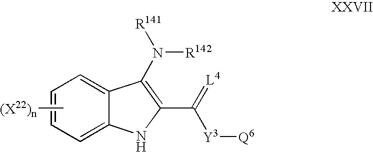
and the pharmaceutically acceptable salts thereof, wherein:
L⁴ is oxygen or sulfur;
Y³ is a direct bond or C₁-C₄ alkylidene;
Q⁶ is:
   (a) C₁-C₆ alkyl or halosubstituted C₁-C₆ alkyl, said alkyl being optionally substituted with up to three substituents independently selected from hydroxyl, C₁-C₄ alkoxy, amino and mono- or di-(C₁-C₄ alkyl)amino,
   (b) C₃-C₇ cycloalkyl optionally substituted with up to three substituents independently selected from hydroxyl, C₁-C₄ alkyl and C₁-C₄ alkoxy,
   (c) phenyl or naphthyl, said phenyl or naphthyl being optionally substituted with up to four substituents independently selected from:
(c-1) halo, C₁-C₄ alkyl, halosubstituted C₁-C₄ alkyl, hydroxyl, C₁-C₄ alkoxy, halosubstituted C₁-C₄ alkoxy, S(O)ₘR¹⁴³, SO₂ NH₂, SO₂ N(C₁-C₄ alkyl)₂, amino, mono- or di-(C₁-C₄ alkyl)amino, NHSO₂R¹⁴³, NHC(O)R¹⁴³, CN, CO₂H, CO₂(C₁-C₄ alkyl), C₁-C₄ alkyl-OH, C₁-C₄ alkyl-OR¹⁴³, CONH₂, CONH(C₁-C₄ alkyl), CON(C₁-C₄ alkyl)₂ and —O—Y-phenyl, said phenyl being optionally substituted with one or two substituents independently selected from halo, C₁-C₄ alkyl, CF₃, hydroxyl, OR¹⁴³, S(O)ₘR¹⁴³ amino, mono- or di-(C₁-C₄ alkyl)amino and CN;
   (d) a monocyclic aromatic group of 5 atoms, said aromatic group having one heteroatom selected from O, S and N and optionally containing up to three N atoms in addition to said heteroatom, and said aromatic group being substituted with up to three substitutents independently selected from:
(d-1) halo, C₁-C₄ alkyl, halosubstituted C₁-C₄ alkyl, hydroxyl, C₁-C₄ alkoxy, halosubstituted C₁-C₄ alkoxy, C₁-C₄ alkyl-OH, S(O)ₘR¹⁴³, SO₂ NH₂, SO₂ N(C₁₋₄ alkyl)₂, amino, mono- or di-(C₁-C₄ alkyl)amino, NHSO₂R¹⁴³, NHC(O)R¹⁴³, CN, CO₂H, CO₂ (C₁-C₄ alkyl), C₁-C₄ alkyl-OR¹⁴³, CONH₂, CONH(C₁-C₄ alkyl), CON(C₁-C₄ alkyl)₂, phenyl, and mono-, di- or tri-substituted phenyl wherein the substituent is independently selected from halo, CF₃, C₁-C₄ alkyl, hydroxyl, C₁-C₄ alkoxy, OCF₃, SR¹⁴³, SO₂ CH₃, SO₂ NH₂, amino, C₁₋₄ alkylamino and NHSO₂R¹⁴³;
   (e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);
R¹⁴¹ is hydrogen or C₁-C₆ alkyl optionally substituted with a substituent selected independently from hydroxyl, OR¹⁴³, nitro, amino, mono- or di-(C₁-C₄ alkyl)amino, CO₂H, CO₂ (C₁-C₄ alkyl), CONH₂, CONH(C₁-C₄ alkyl) and CON(C₁-C₄ alkyl)₂;
R¹⁴² is:
   (a) hydrogen,
   (b) C₁-C₄ alkyl,
   (c) C(O)R¹⁴⁵,
   wherein R¹⁴⁵ is selected from:
(c-1) C₁-C₂₂ alkyl or C₂-C₂₂ alkenyl, said alkyl or alkenyl being optionally substituted with up to four substituents independently selected from:
   (c-1-1) halo, hydroxyl, OR¹⁴³, S(O)ₘR¹⁴³, nitro, amino, mono- or di-(C₁-C₄ alkyl)amino, NHSO₂R¹⁴³, CO₂H, CO₂ (C₁-C₄ alkyl), CONH₂, CONH(C₁-C₄ alkyl), CON(C₁-C₄ alkyl)₂, OC(O)R¹⁴³, thienyl, naphthyl and groups of the following formulas: 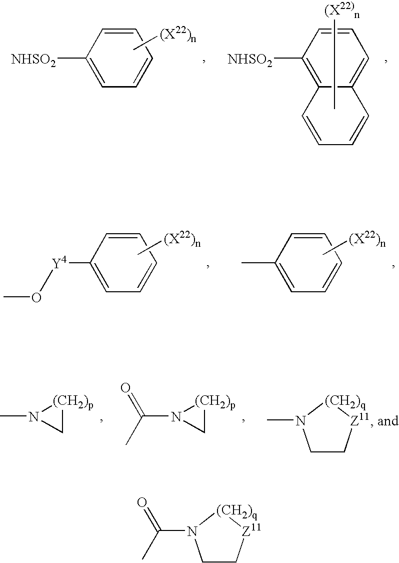
(c-2) C₁-C₂₂ alkyl or C₂-C₂₂ alkenyl, said alkyl or alkenyl being optionally substituted with five to forty-five halogen atoms,
(c-3)—Y⁵—C₃-C₇ cycloalkyl or —Y⁵—C₃-C₇ cycloalkenyl, said cycloalkyl or cycloalkenyl being optionally substituted with up to three substituent independently selected from:
   (c-3-1) C₁-C₄ alkyl, hydroxyl, OR⁴³, S(O)ₘR¹⁴³, amino, mono- or di-(C₁-C₄ alkyl)amino, CONH₂, CONH(C₁-C₄ alkyl) and CON(C₁-C₄ alkyl)₂,
(c-4) phenyl or naphthyl, said phenyl or naphthyl being optionally substituted with up to seven (preferably up to seven) substituents independently selected from:
   (c-4-1) halo, C₁-C₈ alkyl, C₁-C₄ alkyl-OH, hydroxyl, C₁-C₈ alkoxy, halosubstituted C₁-C₈ alkyl, halosubstituted C₁-C₈ alkoxy, CN, nitro, S(O)ₘR¹⁴³, SO₂ NH₂, SO₂ NH(C₁-C₄ alkyl), SO₂ N(C₁-C₄ alkyl)₂, amino, C₁-C₄ alkylamino, di-(C₁-C₄ alkyl)amino, CONH₂, CONH(C₁-C₄ alkyl), CON(C₁-C₄ alkyl)₂, OC(O)R¹⁴³, and phenyl optionally substituted with up to three substituents independently selected from halo, C₁-C₄ alkyl, hydroxyl, OCH₃, CF₃, OCF₃, CN, nitro, amino, mono- or di-(C₁-C₄ alkyl)amino, CO₂H, CO₂ (C₁-C₄ alkyl) and CONH₂,
(c-5) a monocyclic aromatic group as defined in (d) and (e) above, said aromatic group being optionally substituted with up to three substituents independently selected from:
   (c-5-1) halo, C₁-C₈ alkyl, C₁-C₄ alkyl-OH, hydroxyl, C₁-C₈ alkoxy, CF₃, OCF₃, CN, nitro, S(O)ₘR¹⁴³, amino, mono- or di-(C₁-C₄ alkyl)amino, CONH₂, CONH(C₁-C₄ alkyl), CON(C₁-C₄ alkyl)₂, CO₂H and CO₂ (C₁-C₄ alkyl), and —Y-phenyl, said phenyl being optionally substituted with up to three substituents independently selected halogen, C₁-C₄ alkyl, hydroxyl, C₁-C₄ alkoxy, CF₃, OCF₃, CN, nitro, S(O)ₘR¹⁴³, amino, mono- or di-(C₁-C₄ alkyl)amino, CO₂H, CO₂ (C₁-C₄ alkyl), CONH₂, CONH(C₁-C₄ alkyl) and CON(C₁-C₄ alkyl)₂,
(c-6) a group of the following formula: 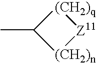
X²² is halo, C₁-C₄ alkyl, hydroxyl, C₁-C₄ alkoxy, halosubstitutued C₁-C₄ alkoxy, S(O)ₘR¹⁴³, amino, mono- or di-(C₁-C₄ alkyl)amino, NHSO₂ R¹⁴³, nitro, halosubstitutued C₁-C₄ alkyl, CN, CO₂H, CO₂ (C₁-C₄ alkyl), C₁-C₄ alkyl-OH, C₁-C₄ alkylOR¹⁴³, CONH₂, CONH(C₁-C₄ alkyl) or CON(C₁-C₄ alkyl)₂;
R¹⁴³ is C₁-C₄ alkyl or halosubstituted C₁-C₄ alkyl;
m is 0, 1 or 2; n is 0, 1, 2 or 3; p is 1, 2, 3, 4 or 5; q is 2 or 3;
Z¹¹ is oxygen, sulfur or NR¹44; and
R¹⁴⁴ is hydrogen, C₁-C₆ alkyl, halosubstitutued C₁-C₄ alkyl or —Y⁵-phenyl, said phenyl being optionally substituted with up to two substituents independently selected from halo, C₁-C₄ alkyl, hydroxyl, C₁-C₄ alkoxy, S(O)ₘR¹⁴³, amino, mono- or di-(C₁-C₄ alkyl)amino, CF₃, OCF₃, CN and nitro;
   with the proviso that a group of formula —Y⁵Q is not methyl or ethyl when
X²² is hydrogen;
L⁴ is oxygen;
R¹⁴¹ is hydrogen; and
R¹⁴² is acetyl.

Aryl phenylhydrazides that are described in U.S. Pat. No. 6,077,869 can serve as Cox-2 selective inhibitors of the present invention. Such aryl phenylhydrazides have the formula shown below in formula XXVIII: 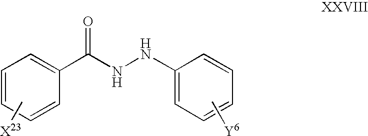
wherein:
X²³ and Y⁶ are selected from hydrogen, halogen, alkyl, nitro, amino, hydroxy, methoxy and methylsulfonyl;
   or a pharmaceutically acceptable salt thereof.

Materials that can serve as a Cox-2 selective inhibitor of the present invention include 2-aryloxy, 4-aryl furan-2-ones that are described in U.S. Pat. No. 6,140,515. Such 2-aryloxy, 4-aryl furan-2-ones have the formula shown below in formula XXIX: 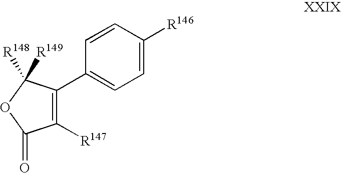
or a pharmaceutical salt thereof, wherein:
R¹⁴⁶ is selected from the group consisting of SCH₃, —S(O)₂ CH₃ and —S(O)₂ NH₂;
R¹⁴⁷ is selected from the group consisting of OR¹⁵⁰, mono or di-substituted phenyl or pyridyl wherein the substituents are selected from the group consisting of methyl, chloro and F;
R¹⁵⁰ is unsubstituted or mono or di-substituted phenyl or pyridyl wherein the substituents are selected from the group consisting of methyl, chloro and F;
R¹⁴⁸ is H, C₁-C₄ alkyl optionally substituted with 1 to 3 groups of F, Cl or Br; and
R¹⁴⁹ is H, C₁-C₄ alkyl optionally substituted with 1 to 3 groups of F, Cl or Br, with the proviso that R¹⁴⁸ and R¹⁴⁹ are not the same.

Materials that can serve as a Cox-2 selective inhibitor of the present invention include bisaryl compounds that are described in U.S. Pat. No. 5,994,379. Such bisaryl compounds have the formula shown below in formula XXX: 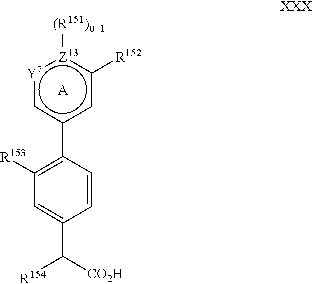
or a pharmaceutically acceptable salt, ester or tautomer thereof, wherein:
Z¹³ is C or N;
when Z¹³ is N, R¹⁵¹ represents H or is absent, or is taken in conjunction with R¹⁵² as described below:
when Z¹³ is C, R¹⁵¹ represents H and R¹⁵² is a moiety which has the following characteristics:
   (a) it is a linear chain of 3-4 atoms containing 0-2 double bonds, which can adopt an energetically stable transoid configuration and if a double bond is present, the bond is in the trans configuration,
   (b) it is lipophilic except for the atom bonded directly to ring A, which is either lipophilic or non-lipophilic, and
   (c) there exists an energetically stable configuration planar with ring A to within about 15 degrees;
or R¹⁵¹ and R¹⁵² are taken in combination and represent a 5- or 6-membered aromatic or non-aromatic ring D fused to ring A, said ring D containing 0-3 heteroatoms selected from O, S and N;
   said ring D being lipophilic except for the atoms attached directly to ring A, which are lipophilic or non-lipophilic, and said ring D having available an energetically stable configuration planar with ring A to within about 15 degrees; said ring D further being substituted with 1 R^{a} group selected from the group consisting of: C₁-C₂ alkyl, —OC₁-C₂ alkyl, —NHC₁-C₂ alkyl, —N(C₁-C₂ alkyl)₂, —C(O)C₁-C₂ alkyl, —S—C₁-C₂ alkyl and —C(S)C₁-C₂ alkyl;
Y⁷ represents N, CH or C—OC₁-C₃ alkyl, and when Z¹³ is N, Y⁷ can also represent a carbonyl group;
R¹⁵³ represents H, Br, Cl or F; and
R¹⁵⁴ represents H or CH₃.

Compounds useful as Cox-2 selective inhibitors of the present invention include 1,5-diarylpyrazoles that are described in U.S. Pat. No. 6,028,202. Such 1,5-diarylpyrazoles have the formula shown below in formula XXXI: 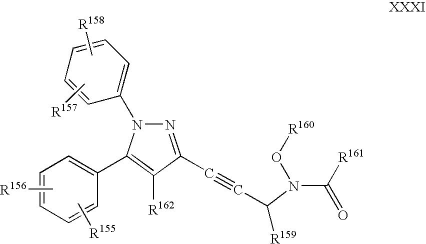
wherein:
R¹⁵⁵, R¹⁵⁶, R¹⁵⁷, and R¹⁵⁸ are independently selected from the groups consisting of hydrogen, C₁-C₅ alkyl, C₁-C₅ alkoxy, phenyl, halo, hydroxyl, C₁-C₅ alkylsulfonyl, C₁-C₅ alkylthio, trihaloC₁-C₅ alkyl, amino, nitro and 2-quinolinylmethoxy;
R¹⁵⁹ is hydrogen, C₁-C₅ alkyl, trihaloC₁-C₅ alkyl, phenyl, substituted phenyl where the phenyl substitutents are halogen, C₁-C₅ alkoxy, trihaloC₁-C₅ alkyl or nitro or R¹⁵⁹ is heteroaryl of 5-7 ring members where at least one of the ring members is nitrogen, sulfur or oxygen;
R¹⁶⁰ is hydrogen, C₁-C₅ alkyl, phenyl C₁-C₅ alkyl, substituted phenyl C₁-C₅ alkyl where the phenyl substitutents are halogen, C₁-C₅ alkoxy, trihaloC₁-C₅ alkyl or nitro, or R¹⁶⁰ is C₁-C₅ alkoxycarbonyl, phenoxycarbonyl, substituted phenoxycarbonyl where the phenyl substitutents are halogen, C₁-C₅ alkoxy, trihaloC₁-C₅ alkyl or nitro;
R¹⁶¹ is C₁-C₁₀ alkyl, substituted C₁-C₁₀ alkyl where the substituents are halogen, trihaloC₁-C₅ alkyl, C₁-C₅ alkoxy, carboxy, C₁-C₅ alkoxycarbonyl, amino, C₁-C₅ alkylamino, diC₁-C₅ alkylamino, diC₁-C₅ alkylaminoC₁-C₅ alkylamino, C₁-C₅ alkylaminoC₁-C₅ alkylamino or a heterocycle containing 4-8 ring atoms where one more of the ring atoms is nitrogen, oxygen or sulfur, where said heterocycle may be optionally substituted with C₁-C₅ alkyl; or R¹⁶¹ is phenyl, substituted phenyl (where the phenyl substitutents are one or more of C₁-C₅ alkyl, halogen, C₁-C₅ alkoxy, trihaloC₁-C₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or
R¹⁶¹ is NR¹⁶³R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁-C₅ alkyl; R¹⁶² is hydrogen, C₁-C₅ alkyl, nitro, amino, and halogen;
   and pharmaceutically acceptable salts thereof.

Materials that can serve as a Cox-2 selective inhibitor of the present invention include 2-substituted imidazoles that are described in U.S. Pat. No. 6,040,320. Such 2-substituted imidazoles have the formula shown below in formula XXXII: 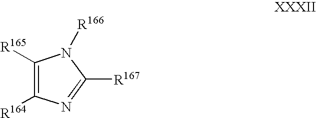
wherein:
R¹⁶⁴ is phenyl, heteroaryl wherein the heteroaryl contains 5 to 6 ring atoms, or
   substituted phenyl;
   wherein the substituents are independently selected from one or members of the group consisting of C₁₋₅ alkyl, halogen, nitro, trifluoromethyl and nitrile;
R¹⁶⁵ is phenyl, heteroaryl wherein the heteroaryl contains 5 to 6 ring atoms,
   substituted heteroaryl;
   wherein the substituents are independently selected from one or more members of the group consisting of C₁-C₅ alkyl and halogen, or substituted phenyl,
   wherein the substituents are independently selected from one or members of the group consisting of C₁-C₅ alkyl, halogen, nitro, trifluoromethyl and nitrile;
R¹⁶⁶ is hydrogen, 2-(trimethylsilyl)ethoxymethyl), C₁-C₅ alkoxycarbonyl, aryloxycarbonyl, arylC₁-C₅ alkyloxycarbonyl, arylC₁-C₅ alkyl, phthalimidoC₁-C₅ alkyl, aminoC₁-C₅ alkyl, diaminoC₁-C₅ alkyl, succinimidoC₁-C₅ alkyl, C₁-C₅ alkylcarbonyl, arylcarbonyl, C₁-C₅ alkylcarbonylC₁-C₅ alkyl, aryloxycarbonylC₁-C₅ alkyl, heteroarylC₁-C₅ alkyl where the heteroaryl contains 5 to 6 ring atoms, or substituted arylC₁-C₅ alkyl,
   wherein the aryl substituents are independently selected from one or more members of the group consisting of C₁-C₅ alkyl, C₁-C₅ alkoxy, halogen, amino, C₁-C₅ alkylamino, and diC₁-C₅ alkylamino;
R¹⁶⁷ is (A¹¹)ₙ-(CH¹⁶⁵)_{q}—X²⁴ wherein:
A¹¹ is sulfur or carbonyl;
n is 0 or 1;
q is 0-9;
X²⁴ is selected from the group consisting of hydrogen, hydroxyl, halogen, vinyl, ethynyl, C₁-C₅ alkyl, C₃-C₇ cycloalkyl, C₁-C₅ alkoxy, phenoxy, phenyl, arylC₁-C₅ alkyl, amino, C₁-C₅ alkylamino, nitrile, phthalimido, amido, phenylcarbonyl, C₁-C₅ alkylaminocarbonyl, phenylaminocarbonyl, arylC₁-C₅ alkylaminocarbonyl, C₁-C₅ alkylthio, C₁-C₅ alkylsulfonyl, phenylsulfonyl,
   substituted sulfonamido,
   wherein the sulfonyl substituent is selected from the group consisting of C₁-C₅ alkyl, phenyl, araC₁-C₅ alkyl, thienyl, furanyl, and naphthyl; substituted vinyl,
   wherein the substituents are independently selected from one or members of the group consisting of fluorine, bromine, chlorine and iodine, substituted ethynyl,
   wherein the substituents are independently selected from one or more members of the group consisting of fluorine, bromine chlorine and iodine, substituted C₁-C₅ alkyl,
   wherein the substituents are selected from the group consisting of one or more C₁-C₅ alkoxy, trihaloalkyl, phthalimido and amino, substituted phenyl,
   wherein the phenyl substituents are independently selected from one or more members of the group consisting of C₁-C₅ alkyl, halogen and C₁-C₅ alkoxy,
   substituted phenoxy,
   wherein the phenyl substituents are independently selected from one or more members of the group consisting of C₁-C₅ alkyl, halogen and C₁-C₅ alkoxy,
   substituted C₁-C₅ alkoxy,
   wherein the alkyl substituent is selected from the group consisting of phthalimido and amino,
   substituted arylC₁-C₅ alkyl,
   wherein the alkyl substituent is hydroxyl, substituted arylC₁-C₅ alkyl,
   wherein the phenyl substituents are independently selected from one or more members of the group consisting of C₁-C₅ alkyl, halogen and C₁-C₅ alkoxy,
   substituted amido,
   wherein the carbonyl substituent is selected from the group consisting of C₁-C₅ alkyl, phenyl, arylC₁-C₅ alkyl, thienyl, furanyl, and naphthyl, substituted phenylcarbonyl,
   wherein the phenyl substituents are independently selected from one or members of the group consisting of C₁-C₅ alkyl, halogen and C₁-C₅ alkoxy,
   substituted C₁-C₅ alkylthio,
   wherein the alkyl substituent is selected from the group consisting of hydroxyl and phthalimido,
   substituted C₁-C₅ alkylsulfonyl,
   wherein the alkyl substituent is selected from the group consisting of hydroxyl and phthalimido,
   substituted phenylsulfonyl,
   wherein the phenyl substituents are independently selected from one or members of the group consisting of bromine, fluorine, chlorine, C₁-C₅ alkoxy and trifluoromethyl,
   with the proviso:
if A¹¹ is sulfur and X²⁴ is other than hydrogen, C₁-C₅ alkylaminocarbonyl, phenylaminocarbonyl, arylC₁-C₅ alkylaminocarbonyl, C₁-C₅ alkylsulfonyl or phenylsulfonyl, then q must be equal to or greater than 1;
if A¹¹ is sulfur and q is 1, then X²⁴ cannot be C₁-C₂ alkyl;
if A¹¹ is carbonyl and q is 0, then X²⁴ cannot be vinyl, ethynyl, C₁-C₅ alkylaminocarbonyl, phenylaminocarbonyl, arylC₁-C₅ alkylaminocarbonyl, C₁-C₅ alkylsulfonyl or phenylsulfonyl;
if A¹¹ is carbonyl, q is 0 and X²⁴ is H, then R¹⁶⁶ is not 2-(trimethylsilyl)ethoxymethyl;
if n is 0 and q is 0, then X²⁴ cannot be hydrogen;
   and pharmaceutically acceptable salts thereof.

Materials that can serve as a Cox-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV: 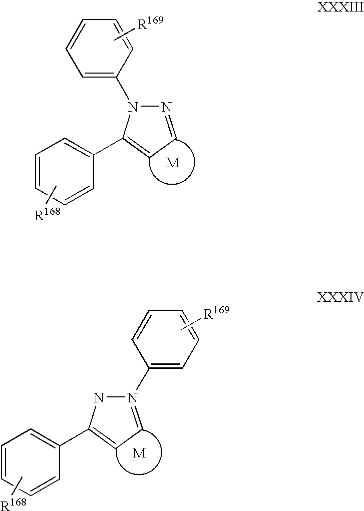
wherein:
R¹⁶⁸ and R¹⁶⁹ are independently selected from the group consisting of hydrogen, halogen, (C₁-C₆)alkyl, (C₁-C₆)alkoxy, nitro, amino, hydroxyl, trifluoro, —S(C₁-C₆)alkyl, —SO(C₁-C₆)alkyl and —SO₂ (C₁-C₆)alkyl; and the fused moiety M is a group selected from the group consisting of an optionally substituted cyclohexyl and cycloheptyl group having the formulae: 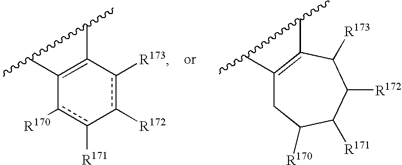
   wherein:
R¹⁷⁰ is selected from the group consisting of hydrogen, halogen, hydroxyl and carbonyl;
or R¹⁷⁰ and R¹⁷¹ taken together form a moiety selected from the group consisting of —OCOCH₂—, —ONH(CH₃)COCH₂—, —OCOCH═and —O—;
R¹⁷¹ and R¹⁷² are independently selected from the group consisting of hydrogen, halogen, hydroxyl, carbonyl, amino, (C₁-C₆)alkyl, (C₁-C₆)alkoxy, ═NOH, —NR¹⁷⁴R¹⁷⁵, —OCH₃, —OCH₂ CH₃, —OSO₂ NHCO₂ CH₃, ═CHCO₂ CH₂ CH₃, —CH₂ CO₂H, —CH₂ CO₂ CH₃, —CH₂ CO₂ CH₂ CH₃, —CH₂ CON(CH₃)₂, —CH₂ CO₂ NHCH₃, —CHCHCO₂ CH₂ CH₃, —OCON(CH₃)OH, —C(COCH₃)₂, di(C₁-C₆)alkyl and di(C₁-C₆)alkoxy;
R¹⁷³ is selected from the group consisting of hydrogen, halogen, hydroxyl, carbonyl, amino, (C₁-C₆)alkyl, (C₁-C₆)alkoxy and optionally substituted carboxyphenyl, wherein substituents on the carboxyphenyl group are selected from the group consisting of halogen, hydroxyl, amino, (C₁-C₆)alkyl and (C₁-C₆)alkoxy;
or R¹⁷² and R¹⁷³ taken together form a moiety selected from the group consisting of —O— and 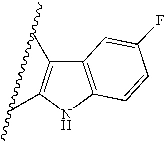
R¹⁷⁴ is selected from the group consisting of hydrogen, OH, —OCOCH₃, —COCH₃ and (C₁-C₆)alkyl; and
R¹⁷⁵ is selected from the group consisting of hydrogen, OH, —OCOCH₃, —COCH₃, (C₁-C₆)alkyl, —CONH₂ and —SO₂ CH₃;
   with the proviso that if M is a cyclohexyl group, then R¹⁷⁰ through R¹⁷³ may not all be hydrogen; and
   pharmaceutically acceptable salts, esters and pro-drug forms thereof.

Esters derived from indolealkanols and novel amides derived from indolealkylamides that are described in U.S. Pat. No. 6,306,890 can serve as Cox-2 selective inhibitors of the present invention. Such compounds have the general formula shown below in formula XXXV: 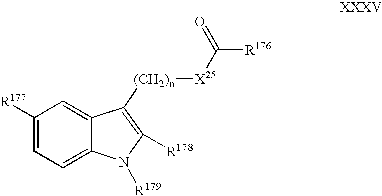
wherein:
R¹⁷⁶ is C₁-C₆ alkyl, C₁-C₆ branched alkyl, C₄-C₈ cycloalkyl, C₁-C₆ hydroxyalkyl, branched C₁-C₆ hydroxyalkyl, hydroxyl substituted C₄-C₈ aryl, primary, secondary or tertiary C₁-C₆ alkylamino, primary, secondary or tertiary branched C₁-C₆ alkylamino, primary, secondary or tertiary C₄-C₈ arylamino, C₁-C₆ alkylcarboxylic acid, branched C₁-C₆ alkylcarboxylic acid, C₁-C₆ alkylester, branched C₁-C₆ alkylester, C₄-C₈ aryl, C₄-C₈ arylcarboxylic acid, C₄-C₈ arylester, C₄-C₈ aryl substituted C₁-C₆ alkyl, C₄-C₈ heterocyclic alkyl or aryl with O, N or S in the ring, alkyl-substituted or aryl-substituted C₄-C₈ heterocyclic alkyl or aryl with O, N or S in the ring, or halo-substituted versions thereof, where halo is chloro, bromo, fluoro or iodo;
R¹⁷⁷ is C₁-C₆ alkyl, C₁-C₆ branched alkyl, C₄-C₈ cycloalkyl, C₄-C₈ aryl, C₄-C₈ aryl-substituted C₁-C₆ alkyl, C₁-C₆ alkoxy, C₁-C₆ branched alkoxy, C₄-C₈ aryloxy, or halo-substituted versions thereof or
R¹⁷⁷ is halo where halo is chloro, fluoro, bromo, or iodo;
R¹⁷⁸ is hydrogen, C₁-C₆ alkyl or C₁-C₆ branched alkyl;
R¹⁷⁹ is C₁-C₆ alkyl, C₄-C₈ aroyl, C₄-C₈ aryl, C₄-C₈ heterocyclic alkyl or aryl with O, N or S in the ring, C₄-C₈ aryl-substituted C₁-C₆ alkyl, alkyl-substituted or aryl-substituted C₄-C₈ heterocyclic alkyl or aryl with O, N or S in the ring, alkyl-substituted C₄₋₈ aroyl, or alkyl-substituted C₄C₈ aryl, or halo-substituted versions thereof where halo is chloro, bromo, or iodo;
n is 1, 2, 3, or 4; and
X²⁵ is O, NH, or N—R¹⁸⁰, where R¹⁸⁰ is C₁-C₆ or C₁-C₆ branched alkyl.

Materials that can serve as a Cox-2 selective inhibitor of the present invention include pyridazinone compounds that are described in U.S. Pat. No. 6,307,047. Such pyridazinone compounds have the formula shown below in formula XXXVI: 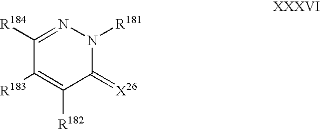
or a pharmaceutically acceptable salt, ester, or prodrug thereof, wherein:
X²⁶ is selected from the group consisting of O, S, —NR¹⁸⁵, —NOR^{a}, and —NNR^{b}R^{c};
R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;
R^{a}, R^{b}, and R^{c} are independently selected from the group consisting of alkyl, aryl, arylalkyl, cycloalkyl, and cycloalkylalkyl;
R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙ C(O)R¹⁸⁶, —(CH₂)ₙ CH(OH)R¹⁸⁶, —(CH₂)ₙ C(NOR^{d})R¹⁸⁶, —(CH₂)ₙ CH(NOR^{d})R¹⁸⁶, —(CH₂)ₙ CH(NR^{d}R^{e})R¹⁸⁶, —R¹⁸⁷ R¹⁸⁸ (CH₂)ₙ C≡CR¹⁸⁸—(CH₂)ₙ [CH(CX^{26′}₃)]ₘ (CH₂)ₚR¹⁸⁸, —(CH₂)ₙ (CX^{26′}₂)ₘ (CH₂)ₚR¹⁸⁸, and —(CH₂)ₙ (CHX^{26′})ₘ (CH₂)ₘR¹⁸⁸;
R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;
R¹⁸⁷ is selected from the group consisting of alkenylene, alkylene, halo-substituted alkenylene, and halo-substituted alkylene;
R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;
R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;
X^{26′} is halogen;
m is an integer from 0-5;
n is an integer from 0-10;
p is an integer from 0-10;
R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴; provided that one of R¹⁸²R¹⁸³ or R¹⁸⁴ must be Z¹⁴, and further provided that only one of R¹⁸², R¹⁸³, or R¹⁸⁴ is Z¹⁴;
Z¹⁴ is selected from the group consisting of: 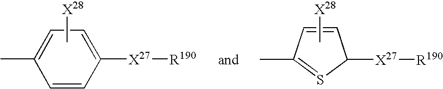
X²⁷ is selected from the group consisting of S(O)₂, S(O)(NR¹⁹¹), S(O), Se(0)₂, P(O)(OR¹⁹²), and P(O)(NR¹⁹³R¹⁹⁴);
X²⁸ is selected from the group consisting of hydrogen, alkenyl, alkyl, alkynyl and halogen;
R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;
R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹¹⁸⁸;
Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, NC(R¹⁹⁷)R¹⁹⁵, and —N(R¹⁹⁷)R¹⁹⁵;
R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹R²⁰⁰; and
R¹⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Benzosulphonamide derivatives that are described in U.S. Pat. No. 6,004,948 are useful as Cox-2 selective inhibitors of the present invention. Such benzosulphonamide derivatives have the formula shown below in formula XXXVII: 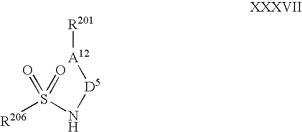
wherein:
A¹² denotes oxygen, sulphur or NH;
R²⁰¹ denotes a cycloalkyl, aryl or heteroaryl group optionally mono- or polysubstituted by halogen, alkyl, CF₃ or alkoxy;
D⁵ denotes a group of formula XXXVIII or XXXIX: 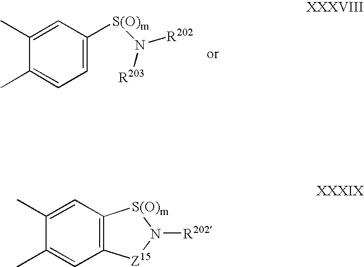
R²⁰² and R²⁰³ independently of each other denote hydrogen, an optionally polyfluorinated alkyl radical, an aralkyl, aryl or heteroaryl radical or a radical (CH₂)ₙ-X²⁹; or
R²⁰² and R²⁰³ together with the N-atom denote a three- to seven-membered, saturated, partially or totally unsaturated heterocycle with one or more heteroatoms N, O, or S, which may optionally be substituted by oxo, an alkyl, alkylaryl or aryl group or a group (CH₂)ₙ-X²⁹, R²⁰²′ denotes hydrogen, an optionally polyfluorinated alkyl group, an aralkyl, aryl or heteroaryl group or a group (CH₂)ₙ-X²⁹,
   wherein:
X²⁹ denotes halogen, NO₂, —OR²⁰⁴, —COR²⁰⁴, —CO₂R²⁰⁴, —OCO₂R²⁰⁴, —CN, —CONR²⁰⁴ OR²⁰⁵, —CONR²⁰⁴R²⁰⁵, —SR²⁰⁴ S(O)R²⁰⁴, —S(O)₂R²⁰⁴, —NR²⁰⁴R²⁰⁵, —NHC(O)R²⁰⁴, —NHS(O)R²⁰⁴;
Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂ NH—, —N═CH—, —NHCH—, —CH₂—CH₂—NH—, —CH═CH—, >N—R²⁰³, >C═O, >S(O)ₘ;
R²⁰⁴ and R²⁰⁵ independently of each other denote hydrogen, alkyl, aralkyl or aryl;
n is an integer from 0 to 6;
R²⁰⁶ is a straight-chained or branched C₁-C₄ alkyl group which may optionally be mono- or polysubstituted by halogen or alkoxy, or R²⁰⁶ denotes CF₃; and
m denotes an integer from 0 to 2;
   with the proviso that A¹² does not represent 0 if R²⁰⁶ denotes CF₃;
   and the pharmaceutically acceptable salts thereof.

Materials that can serve as Cox-2 selective inhibitors of the present invention include methanesulfonyl-biphenyl derivatives that are described in U.S. Pat. No. 6,583,321. Such methanesulfonyl-biphenyl derivatives have the formula shown below in formula XL: 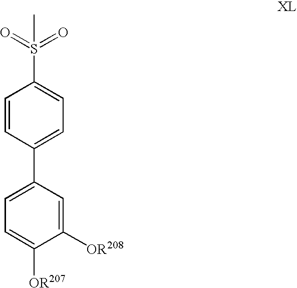
wherein:
R²⁰⁷ and R²⁰⁸ are respectively a hydrogen;
C₁-C₄-alkyl substituted or not substituted by halogens;
C₃-C₇-cycloalkyl;
C₁-C₅-alkyl containing 1-3 ether bonds and/or an aryl substitute;
substituted or not substituted phenyl;
or substituted or not substituted five or six ring-cycled heteroaryl containing more than one hetero atoms selected from a group consisting of nitrogen, sulfur, and oxygen (wherein phenyl or heteroaryl can be one- or multi-substituted by a substituent selected from a group consisting of hydrogen, methyl, ethyl, and isopropyl).

Cox-2 selective inhibitors such as 1H-indole derivatives described in U.S. Pat. No. 6,599,929 are useful in the present invention. Such 1H-indole derivatives have the formula shown below in formula XLI: 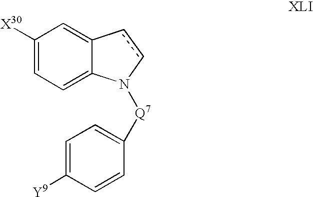
wherein:
X³⁰ is —NHSO₂R²⁰⁹ wherein R²⁰⁹ represents hydrogen or C₁-C₃-alkyl;
Y⁹ is hydrogen, halogen, C₁-C₃-alkyl substituted or not substituted by halogen, NO₂, NH₂, OH, OMe, CO₂H, or CN; and
Q⁷ is C═O, C═S, or CH₂.

Compounds that are useful as Cox-2 selective inhibitors of the present invention include prodrugs of Cox-2 inhibitors that are described in U.S. Pat. Nos. 6,436,967 and 6,613,790. Such prodrugs of Cox-2 inhibitors have the formula shown below in formula XLII: 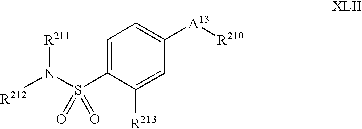
wherein:
A¹³ is a ring substituent selected from partially unsaturated heterocyclic, heteroaryl, cycloalkenyl and aryl, wherein A¹³ is unsubstituted or substituted with one or more radicals selected from alkylcarbonyl, formyl, halo, alkyl, haloalkyl, oxo, cyano, nitro, carboxyl, alkoxy, aminocarbonyl, alkoxycarbonyl, carboxyalkyl, cyanoalkyl, hydroxyalkyl, haloalkylsulfonyloxy, alkoxyalkyloxyalkyl, carboxyalkoxyalkyl, cycloalkylalkyl, alkenyl, alkynyl, heterocycloxy, alkylthio, cycloalkyl, aryl, heterocyclyl, cycloalkenyl, aralkyl, heterocyclylalkyl, alkylthioalkyl, arylcarbonyl, aralkylcarbonyl, aralkenyl, alkoxyalkyl, arylthioalkyl, aryloxyalkyl, aralkylthioalkyl, araalkoxyalkyl, alkoxycarbonylalkyl, aminocarbonylalkyl, alkylaminocarbonyl, N-arylaminocarbonyl, N-alkyl-N-arylaminocarbonyl, alkylaminocarbonylalkyl, alkylamino, -arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl;
R²¹⁰ is selected from heterocyclyl, cycloalkyl, cycloalkenyl, and aryl, wherein R²¹⁰ is unsubstituted or substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy, and alkylthio;
R²¹¹ is selected from hydrido and alkoxycarbonylalkyl;
R²¹² is selected from alkyl, carboxyalkyl, acyl, alkoxycarbonyl, heteroarylcarbonyl, alkoxycarbonylalkylcarbonyl, alkoxycarbonylcarbonyl, amino acid residue, and alkylcarbonylaminoalkylcarbonyl;
provided A¹³ is not tetrazolium, or pyridinium; and further provided A¹³ is not indanone when R²¹² is alkyl or carboxyalkyl; further provided A¹³ is not thienyl, when R²¹⁰ is 4-fluorophenyl, when R²¹¹ is hydrido, and when R²¹² is methyl or acyl; and
R²¹³ is hydrido;
   or a pharmaceutically acceptable salt thereof.

Specific non-limiting examples of substituted sulfonamide prodrugs of Cox-2 inhibitors disclosed in U.S. Pat. No. 6,436,967 that are useful in the present invention include:
N-[[4-[3-(difluoromethyl)-5-(3-fluoro-4-methoxyphenyl)-1H-pyrazol-1-yl]phenyl]sulfonyl]propanamide;
N-[[4-[3-(difluoromethyl)-5-(3-fluoro-4-methoxyphenyl)-1H-pyrazol-1-yl]phenyl]sulfonyl]butanamide;
N-[[4-[1,5-dimethyl)-3-phenyl-1H-pyrazol-4-yl]phenyl]sulfonyl]acetamide;
N-[[4-(2-(3-pyridinyl)-4-(trifluoromethyl)-1H-imidazol-1-yl)phenyl]sulfonyl]acetamide;
N-[[4-[2-(5-methylpyridin-3-yl)-4-(trifluoromethyl)-1H-imidazol-1-yl]phenyl]sulfonyl]acetamide;
N-[[4-[2-(2-methylpyridin-3-yl)-4-(trifluoromethyl)-1H-imidazol-1-yl]phenyl]sulfonyl]acetamide;
N-[[4-[2-(5-methylpyridin-3-yl)-4-(trifluoromethyl)-1H-imidazol-1-yl]phenyl]sulfonyl]butanamide;
N-[[4-[2-(2-methylpyridin-3-yl)-4-(trifluoromethyl)-1H-imidazol-1-yl]phenyl]sulfonyl]butanamide;
N-[[4-[2-(3-chloro-5-methylphenyl)-4-(trifluoromethyl)-1H-imidazol-1-yl]phenyl]sulfonyl]acetamide;
N-[[4-[3-(3-fluorophenyl)-5-methylisoxazol-4-yl]phenyl]sulfonyl]acetamide;
2-methyl-N-[[4-(5-methyl-3-phenylisoxazol-4-yl)phenyl]sulfonyl]propanamide;
N-[[4-(5-methyl-3-phenylisoxazol-4-yl)phenyl]sulfonyl]propanamide;
N-[[4-(5-methyl-3-phenylisoxazol-4-yl)phenyl]sulfonyl]benzamide;
2,2-dimethyl-N-[[4-(5-methyl-3-phenylisoxazol-4-yl)phenyl]sulfonyl]propanamide;
N-[[4-5-methyl-3-phenylisoxazol-4-yl)phenyl]sulfonyl]butanamide;
N-[[4-(5-methyl-3-phenylisoxazol-4-yl)phenyl]sulfonyl]pentanamide;
N-[[4-(5-methyl-3-phenylisoxazol-4-yl)phenyl]sulfonyl]hexanamide;
3-methoxy-N-[[4-(5-methyl-3-phenylisoxazol-4-yl)phenyl]sulfonyl]propanamide;
2-ethoxy-N-[[4-(5-methyl-3-phenylisoxazol-4-yl)phenyl]sulfonyl]acetamide;
N-[[4-[5-methyl-3-phenylisoxazol-4-yl]phenyl]sulfonyl]acetamide;
N-[[4-[5-(4-chlorophenyl)-3-(trifluoromethyl)-1H pyrazol-1-yl]phenyl]sulfonyl]propanamide;
N-[[4-[5-(4-chlorophenyl)-3-(trifluoromethyl)-1H-pyrazol-1-yl]phenyl]sulfonyl]butanamide;
N-[[4-[5-(4-chlorophenyl)-3-(trifluoromethyl)-1H-pyrazol-1-yl]phenyl]sulfonyl]acetamide;
N-[[4-[3-(difluoromethyl)-6-fluoro-1,5-dihydro-7-methoxy-[2]benzothiopyrano [4,3-c]pyrazol-1-yl)phenyl]sulfonyl]acetamide;
N-[[4-[6-fluoro-1,5-dihydro-7-methoxy-3-(trifluoromethyl)-[2]benzothiopyran o[4,3-c]pyrazol-1-yl]phenyl]sulfonyl]acetamide;
N-[[4-[3-(difluoromethyl)-5-(3-fluoro-4-methoxyphenyl)-1H-pyrazol-1-yl]phenyl]sulfonyl]acetamide;
N-[[4-(2-methyl-4-phenyloxazol-5-yl)phenyl]sulfonyl]acetamide;
methyl[[[4-(5-methyl-3-phenylisoxazol-4-yl)phenyl]sulfonyl]amino]oxoacetate;
2-methoxy-N-[[4-(5-methyl-3-phenylisoxazol-4-yl)phenyl]sulfonyl]acetamide;
N-[[4-[5-(difluoromethyl)-3-phenylisoxazol-4-yl]phenyl]sulfonyl]propanamide;
N-[[4-[5-(difluoromethyl)-3-phenylisoxazol-4-yl]phenyl]sulfonyl]butanamide;
N-[[4-(5-methyl-3-phenylisoxazol-4-yl)phenyl]sulfonyl]formamide;
1,1-dimethylethyl-N-[[4-(5-methyl-3-phenylisoxazol-4-yl)phenyl]sulfonyl]carbamate;
N-[[.sup.4-(5-methyl-3-phenylisoxazol-4-yl)phenyl]sulfonyl]glycine;
2-amino-N-[[4-(5-methyl-3-phenylisoxazol-4-yl)phenyl]sulfonyl]acetamide;
2-(acetylamino)-N-[[4-(5-methyl-3-phenylisoxazol-4-yl)phenyl]sulfonyl]acetamide;
methyl 4-[[[4-(5-methyl-3-phenylisoxazol-4-yl)phenyl]sulfonyl]amino]-4-oxobutanoate;
methyl N-[[4-(5-methyl-3-phenylisoxazol-4-yl)phenyl]sulfonyl]carbamate;
N-acetyl-N-[[4-(5-methyl-3-phenylisoxazol-4-yl)phenyl]sulfonyl]glycine,
ethyl ester;
N-[[4-(5-(4-methylphenyl)-3-(trifluoromethyl)-1H-pyrazol-1-yl)phenyl]sulfonyl]acetamide;
methyl 3-[[[4-(5-methyl-3-phenylisoxazol-4-yl)phenyl]sulfonyl]amino]-3-oxopropanoate;
4-[5-(3-bromo-5-fluoro-4-methoxyphenyl)-2-(trifluoromethyl)oxazol-4-yl]-N-methylbenezenesulfonamide;
N-(1,1-dimethylethyl)-4-(5-methyl-3-phenylisoxazol-4-yl)benzenesulfonamide;
4-[5-(4-fluorophenyl)-3-(trifluoromethyl)-1H-pyrazol-1-yl]-N-methylbenzenesulfonamide;
N-methyl-4-(5-methyl-3-phenylisoxazol-4-yl)benezenesulfonamide;
N-[[4-[5-(hydroxymethyl)-3-phenylisoxazol-4-yl]phenyl]sulfonyl]acetamide
N-[[4-[5-(acetoxymethyl)-3-phenylisoxazol-4-yl]phenyl]sulfonyl]acetamide;
N-[[4-[2-(3-chloro-4-fluorophenyl)cyclopenten-1-yl)phenyl]sulfonyl]acetamide;
4-[2-(4-fluorophenyl)-1H-pyrrol-1-yl]-N-methylbenzenesulfonamide;
N-[[4-(3,4-dimethyl-1-phenyl-1H-pyrazol-5-yl]phenyl]sulfonyl]propanamide;
N-[[4-[2-(2-methylpyridin-3-yl)-4-trifluoromethylimidazol-1-yl]phenyl]sulfonyl]propanamide;
4-[2-(4-fluorophenyl)cyclopenten-1-yl]-N-methylbenezenesulfonamide; and
N-[[4-(3-phenyl-2,3-dihydro-2-oxofuran-4-yl)phenyl]sulfonyl]propanamide.

Those prodrugs disclosed in U.S. Pat. No. 6,613,790 have the general formula shown above in formula XLII wherein:
A¹³ is a pyrazole group optionally substituted at a substitutable position with one or more radicals independently selected at each occurrence from the group consisting of alkylcarbonyl, formyl, halo, alkyl, haloalkyl, oxo, cyano, intro, carboxyl, alkoxy, aminocarbonyl, alkoxycarbonyl, carboxyalkyl, cyanoalkyl, hydroxyalkyl, haloalkylsulonyloxy, alkoxyalkyloxyalkyl, carboxyalkoxyalkyl, alkenyl, alkynyl, alkylthio, alkylthioalkyl, alkoxyalkyl, alkoxycarbonylalkyl, aminocarbonylalkyl, alkylaminocarbonyl, alkylaminocarbonylalkyl, alkylamino, aminoalkyl, alkylaminoalkyl, alkylsutfinyl, alkylsulfonyl, aminosulfonyl, and alkylaminosulfonyl;
R²¹⁰ is a phenyl group optionally substituted at a substitutable position with one or more radicals independently selected at each occurrence from the group consisting of alkyl, haloalkyl, cyano, carboxyl, alkoxycarbonyl, hydroxyl, hydroxyalkyl, haloalkoxy, amino, alkylamino, nitro, alkoxyalkyl, alkylsulfinyl, halo, alkoxy, and alkylthio;
R²¹¹ and R²¹² are independently selected from the group consisting of hydroxyalkyl and hydrido but at least one of R²¹¹ and R²¹² is other than hydrido; and
R²¹³ is selected from the group consisting of hydrido and fluoro.

Examples of prodrug compounds disclosed in U.S. Pat. No. 6,613,790 that are useful as Cox-2 inhibitors of the present invention include, but are not limited to, N-(2-hydroxyethyl)-4-[5-(4-methylphenyl)-3-(trifluoromethyl)-1H-pyrazol-1-yl]benzenesulfonamide, N,N-bis(2-hydroxyethyl)-4-[5-(4-methylphenyl)-3-(trifluoromethyl)-1H-pyrazol-1-yl]benzenesulfonamide, or pharmaceuticaly-acceptable salts thereof.

Cox-2 selective inhibitors such as sulfamoylheleroaryl pyrazole compounds that are described in U.S. Pat. No. 6,583,321 may serve as Cox-2 inhibitors of the present invention. Such sulfamoylheleroaryl pyrazole compounds have the formula shown below in formula XLIII: 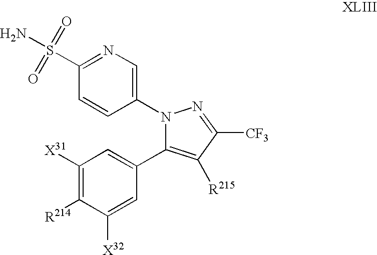
wherein:
R²¹⁴ is furyl, thiazolyl or oxazolyl;
R²¹⁵ is hydrogen, fluoro or ethyl; and
X³¹ and X³² are independently hydrogen or chloro.

Heteroaryl substituted amidinyl and imidazolyl compounds such as those described in U.S. Pat. No. 6,555,563 are useful as Cox-2 selective inhibitors of the present invention. Such heteroaryl substituted amidinyl and imidazolyl compounds have the formula shown below in formula XLIV 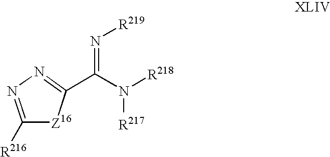
wherein:
Z¹⁶ is O or S,
R²¹⁶ is optionally substituted aryl,
R²¹⁷ is aryl optionally substituted with aminosulfonyl, and
R²¹⁸ and R²¹⁹ cooperate to form an optionally substituted 5-membered ring.

Materials that can serve as Cox-2 selective inhibitors of the present invention include substituted hydroxamic acid derivatives that are described in U.S. Pat. Nos. 6,432,999, 6,512,121, and 6,515,014. These compounds also act as inhibitors of the lipoxygenase-5 enzyme. Such substituted hydroxamic acid derivatives have the general formulas shown below in formulas XLV and XLVI:

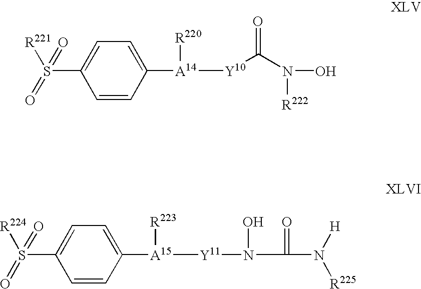

Pyrazole substituted hydroxamic acid derivatives described in U.S. Pat. No. 6,432,999 have the formula shown above in formula XLV, wherein:
A¹⁴ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;
Y¹⁰ is selected from lower alkenylene and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl; or a pharmaceutically acceptable salt thereof.

Pyrazole substituted hydroxamic acid derivatives described in U.S. Pat. No. 6,432,999 may also have the formula shown above in formula XLVI, wherein:
A¹⁵ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;
Y¹¹ is selected from lower alkylene, lower alkenylene and lower alkynylene;

R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;
R²²⁴ is selected from lower alkyl and amino; and
R²²⁵ is selected from hydrido, lower alkyl;
   or a pharmaceutically acceptable salt thereof.

Heterocyclo substituted hydroxamic acid derivatives described in U.S. Pat. No. 6,512,121 have the formula shown above in formula XLV, wherein:
A¹⁴ is a ring substiuent selected from oxazolyl, furyl, pyrrolyl, thiazolyl, imidazolyl, isochiazolyl, isoxazolyl, cyclopentenyl, phenyl, and pyridyl; wherein A¹⁴ is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;
Y¹⁰ is lower alkylene, lower alkenylene, and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl; or a pharmaceutically acceptable salt thereof.

Heterocyclo substituted hydroxamic acid derivatives described in U.S. Pat. No. 6,512,121 may also have the formula shown above in formula XLVI, wherein:
A¹⁵ is a ring substituent selected from oxazolyl, furyl, pyrrolyl, thiazolyl, imidazolyl, isothiazolyl, isoxazolyl, cyclopentenyl, phenyl, and pyridyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarboryl, lower carboxyalkyl, lower cyanoalkyl, and lower hydroxyalkyl;
Y¹¹ is selected from lower alkyl, lower alkenyl and lower alkynyl;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitto, lower alkoxyalkyl, lower alkylsulfinyl, halo, lower alkoxy and lower alkylthio;
R²²⁴ is selected from lower alkyl and amino; and
R²²⁵ is selected from hydrido and alkyl; or a pharmaceutically acceptable salt thereof.

Thiophene substituted hydroxamic acid derivatives described in U.S. Pat. No. 6,515,014 have the formula shown above in formula XLV, wherein:
A¹⁴ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;
Y¹⁰ is ethylene, isopropylene, propylene, butylene, lower alkenylene, and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl; or a pharmaceutically acceptable salt thereof.

Thiophene substituted hydroxamic acid derivatives described in U.S. Pat. No. 6,515,014 may also have the formula shown above in formula XLV, wherein:
A¹⁵ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;
Y¹¹ is selected from lower alkyl, lower alkenyl and lower alkynyl;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²⁴ is selected from lower alkyl and amino; and
R²²⁵ is selected from hydrido and alkyl; or a pharmaceutically acceptable salt thereof.

Compounds that are useful as Cox-2 selective inhibitors of the present invention include pyrazolopyridine compounds that are described in U.S. Pat. No. 6,498,166. Such pyrazolopyridine compounds have the formula shown below in formula XLVII: 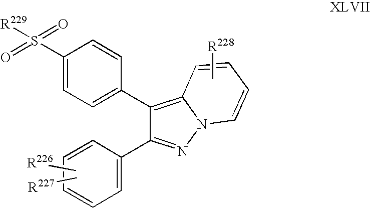
wherein:
R²²⁶ and R²²⁷ are independently selected from the group consisting of H, halogen, C₁-C₆ alkyl, C₁-C₆ alkoxy, and C₁-C₆ alkoxy substituted by one or more fluorine atoms;
R²²⁸ is halogen, CN, CON R²³⁰R²³¹, CO₂H, CO₂ C₁-C₆ alkyl, or NHSO₂R²³⁰;
R²²⁹ is C₁-C₆ alkyl or NH₂; and
R²²⁵ and R²²⁵ are independently selected from the group consisting of H, C₁-C₆ alkyl, phenyl, phenyl substituted by one or more atoms or groups selected from the group consisting of halogen, C₁-C₆ alkyl, C₁-C₆ alkoxy, and C₁-C₆ alkoxy substituted by one or more fluorine atoms, or a pharmaceutically acceptable salt, solvate, ester, or salt or solvate of such ester thereof.

Materials that are useful as Cox-2 selective inhibitors of the present invention include 4,5-diaryl-3(2H)-furanone derivatives that are described in U.S. Pat. No. 6,492,416. Such 4,5-diaryl-3(2H)-furanone derivatives have the formula shown below in formula XLVIII: 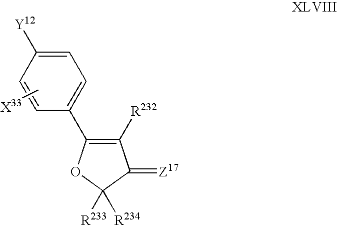
wherein:
X³³ represents halo, hydrido, or alkyl;
Y¹² represents alkylsulfonyl, aminosulfonyl, alkylsulfinyl, (N-acylamino)-sulfonyl, (N-alkylamino)sulfonyl, or alkylthio;
Z¹⁷ represents oxygen or sulfur atom;
R²³³ and R²³⁴ are selected independently from lower alkyl radicals; and R²³² represents a substituted or non-substituted aromatic group of 5 to 10 atoms;
   or a pharmaceutically acceptable salt thereof.

Cox-2 selective inhibitors that can be used in the present invention include 2-phenyl-1,2-benzisoselenazol-3(2H)-one derivatives and 2-phenylcarbomyl-phenylselenyl derivatives that are described in U.S. Pat. No. 6,492,416. Such 2-phenyl-1,2-benzisoselenazol-3(2H)-one derivatives and 2-phenylcarbomyl-phenylselenyl derivatives have the formulas shown below in formulas XLIX or XLIX′: 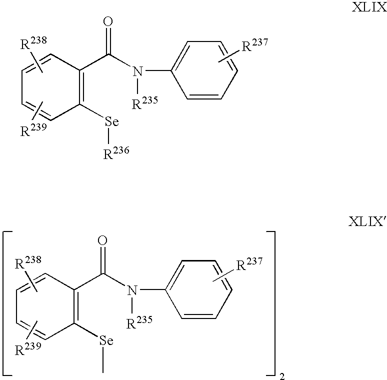
wherein:
R²³⁵ is a hydrogen atom or an alkyl group having 1-3 carbon atoms;
R²³⁶ is a hydrogen atom, a hydroxyl group, an organothiol group that is bound to the selenium atom by its sulfur atom, or R²³⁵ and R²³⁶ are joined to each other by a single bond;
R²³⁷ is a hydrogen atom, a halogen atom, an alkyl group having 1-3 carbon atoms, an alkoxyl group having 1-3 carbon atoms, a trifluoromethyl group, or a nitro group;
R²³⁸ and R²³⁹ are identical to or different from each other, and each is a hydrogen atom, a halogen atom, an alkoxyl group having 1-4 carbon atoms, a trifluoromethyl group, or R²³⁸ and R²³⁹ are joined to each other to form a methylenedioxy group,
   a salt thereof, or a hydrate thereof.

Pyrones such as those disclosed in U.S. Pat. No. 6,465,509 are also useful as Cox-2 inhibitors of the present invention. These pyrone compounds have the general formula shown below in formula L: 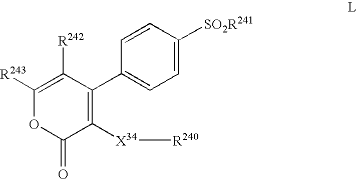
wherein:
X³⁴ is selected from the group consisting of:
   (a) a bond,
   (b) —(CH₂)ₘ—, wherein m 1 or 2,
   (c) —C(O)—,
   (d) —O—,
   (e) —S—, and
   (f) —N(R²⁴⁴)—;
R²⁴⁰ is selected from the group consisting of:
   (a) C₁-C₁₀ alkyl, optionally substituted with 1-3 substituents independently selected from the group consisting of: hydroxy, halo, C₁-C₁₀ alkoxy, C₁-C₁₀ alkylthio, and CN,
   (b) phenyl or naphthyl, and
   (c) heteroaryl, which is comprised of a monocyclic aromatic ring of 5 atoms having one hetero atom which is S, O or N, and optionally 1, 2, or 3 additional N atoms; or
   a monocyclic ring of 6 atoms having one hetero atom which is N, and optionally 1, 2, or 3 additional N atoms, wherein groups (b) and (c) above are each optionally substituted with 1-3 substituents independently selected from the group consisting of: halo, C₁-C₁₀ alkoxy, C₁-C₁₀ alkylthio, CN, C₁-C₁₀ alkyl, optionally substituted to its maximum with halo, and N₃;
R²⁴¹ is selected from the group consisting of
   (a) C₁-C₆ alkyl, optionally substituted to its maximum with halo,
   (b) NH₂, and
   (c) NHC(O)C₁-C₁₀ alkyl, optionally substituted to its maximum with halo;
R²⁴² and R²⁴³ are each independently selected from the group consisting of: hydrogen, halo, and C₁-C₆ alkyl, optionally substituted to its maximum with halo; and
R²⁴⁴ is selected from the group consisting of: hydrogen and C₁-C₆ alkyl, optionally substituted to its maximum with halo.

Examples of pyrone compounds that are useful as Cox-2 selective inhibitors of the present invention include, but are not limited to:
4-(4-Methylsulfonyl)phenyl-3-phenyl-pyran-2-one,
3-(4-Fluorophenyl)-6-methyl-4-(4-methylsulfonyl)phenyl-pyran-2-one,
3-(3-Fluorophenyl)-6-methyl-4-(4-methylsulfonyl)phenyl-pyran-2-one,
6-Methyl-4-(4-methylsulfonyl)phenyl-3-phenyl-pyran-2-one,
6-Difluoromethyl-4-(4-methylsulfonyl)phenyl-3-phenyl-pyran-2-one,
6-Fluoromethyl-4-(4-methylsulfonyl)phenyl-3-phenyl-pyran-2-one,
6-Methyl-4-(4-methylsulfonyl)phenyl-3-phenylthio-pyran-2-one,
6-Methyl-4-(4-methylsulfonyl)phenyl-3-phenoxy-pyran-2-one,
6-Methyl-4-(4-methylsulfonyl)phenyl-3-pyridin-3-yl-pyran-2-one,
3-Isopropylthio-6-methyl-4-(4-methylsulfonyl)phenyl-pyran-2-one,
4-(4-Methylsulfonyl)phenyl)-3-phenylthio-6-trifluoromethyl-pyran-2-one,
3-Isopropylthio-4-(4-methylsulfonyl)phenyl-6-trifluoromethyl-pyran-2-one,
4-(4-Methylsulfonyl)phenyl-3-phenyl-6-(2,2,2-trifluoroethyl)-pyran-2-one, and
3-(3-Hydroxy-3-methylbutyl)-6-methyl-4-(4-methylsulfonyl)phenyl-pyran-2-one.

Organically synthesized or purified from plant sources, free-B-ring flavanoids such as those described in U.S. Published Application No. 2003/0165588, are useful as Cox-2 selective inhibitors of the present invention. Such free-B-ring flavanoids have the general structure shown in formula LI: 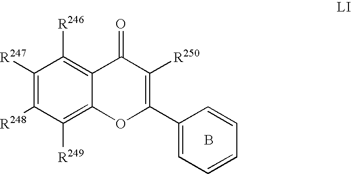
wherein:
R²⁴⁶, R²⁴⁷, R²⁴⁸, R²⁴⁹, and R²⁵⁰ are independently selected from the group consisting of: —H, —OH, —SH, —OR, —SR, —NH₂, —NHR²⁴⁵, —N(R²⁴⁵)₂, —N(R²⁴⁵)₃⁺X³⁵⁻, a carbon, oxygen, nitrogen or sulfur, glycoside of a single or a combination of multiple sugars including, aldopentoses, methyl-aldopentose, aldohexoses, ketohexose and their chemical derivatives thereof; wherein R²⁴⁵ is an alkyl group having between 1-10 carbon atoms; and X³⁵ is selected from the group of pharmaceutically acceptable counter anions including, hydroxyl, chloride, iodide, sulfate, phosphate, acetate, fluoride and carbonate.

Heterocyclo-alkylsulfonyl pyrazoles such as those described in European Patent Application No. EP 1312367 are useful as Cox-2 selective inhibitors of the present invention. Such heterocyclo-alkylsulfonyl pyrazoles have the general formula shown below in formula LII: 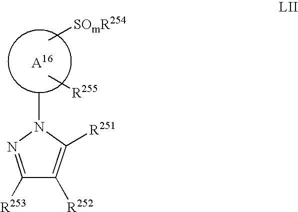
or a pharmaceutically acceptable salt thereof, wherein:
the ring of the formula (R²⁵⁵)-A-(SOₘR²⁵⁴) is selected from the group consisting of: 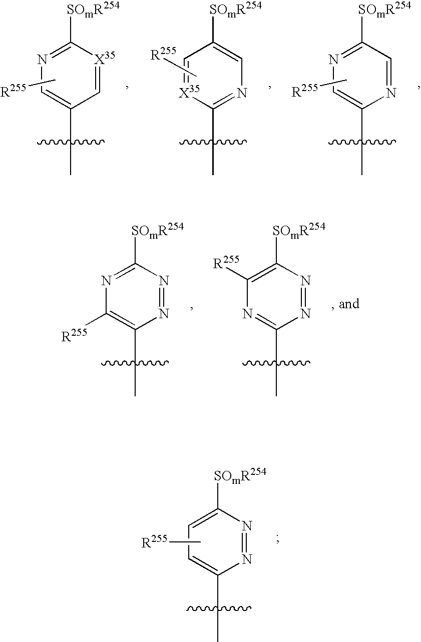
m is 0, 1 or 2;
X³⁵ is >CR²⁵⁵ or >N;
R²⁵¹ is a radical selected from the group consisting of H, NO₂, CN, (C₁-C₆)alkyl, (C₁-C₆)alkyl-SO₂—, (C₆-C₁₀)aryl-SO₂—, H—(C═O)—, (C₁ —C₆)alkyl-(C═O)—, (C₁-C₆)alkyl-)-(C═O)—, (C₁-C₉)heteroaryl-(C═O)—, (C₁-C₉)heterocyclyl-(C═O)—, H₂N—(C═O)—, (C₁-C₆)alkyl-NH—(C═O)—, [(C₁-C₆)alkyl]₂—N—(C═O)—, [(C₆-C₁₀)aryl]₂—NH—(C═O)—, [(C₁-C₆)alkyl]-[((C₆-C₁₀)aryl-N]-(C═O)—, HO—NH—(C═O)—, and (C₁-C₆)alkyl-O—NH—(C═O)—; R²⁵² is a radical selected from the group consisting of H, —NO₂, —CN, (C₂-C₆)alkenyl, (C₂-C₆)alkynyl, (C₃-C₇)cycloalkyl, (C₆-C₁₀)aryl, (C₁-C₉)heteroaryl, (C₁-C₉)heterocyclyl, (C₁-C₆)alkyl-O—, (C₃-C₇)cycloalkyl-O—, (C₆-C₁₀)aryl-O—, (C₁-C₉)heteroaryl-O—, (C₆-C₉)heterocyclyl-O—, H—(C═O)—, (C₁-C₆)alkyl-(C═O)—, (C₃-C₇)cycloalkyl-(C═O)—, (C₆-C₁₀)aryl-(C═O)—, (C₁-C₉)heteroaryl-(C═O)—, (C₁-C₉)heterocyclyl-(C═O)—, (C₁-C₆)alkyl-O—(C═O)—, (C₃-C₇)cycloalkyl-O—(C═O)—, (C₆-C₁₀)aryl-O—(C═O)—, (C₁-C₉)heteroaryl-O—(C═O)—, (C₁-C₉)heterocyclyl-O—(C═O)—, (C₁-C₆)alkyl-(C═O)—O—, (C₃-C₇)cycloalkyl-(C═O)—O—, (C₆-C₁₀)aryl-(C═O)—O—, (C₁-C₉)heteroaryl-(C═O)-O—, (C₁-C₉)heterocyclyl-(C═O)—O—, (C₁-C₆)alkyl-(C═O)—NH—, (C₃-C₇)cycloalkyl-(C=0)—NH—, (C₆-C₁₀aryl-(C═O)—NH—. (C₁-C₉)heteroaryl-(C═O)—NH—, (C₁-C₉)heterocyclyl-(C═O)—NH—, (C₁-C₆)alkyl-O—(C═O)—NH—, (C₁-C₆)alkyl-NH, [(C₁-C₆)alkyl]₂—N—, (C₃-C₇)cycloalkyl-NH—. [(C₃-C₇)cycloalkyl]₂—N—, [(C₆-C₁₀)aryl]-NH—, [(C₆-C₁₀)aryl]₂—N—, [(C₁-C₆)alkyl]-[((C₆-C₁₀)aryl)-N]—, [(C₁-C₉)heteroaryl]-NH—, [(C₁-C₉)heteroaryl]₂—N—, [(C₁-C₉)heterocycly]-NH—, [(C₁-C₉)heterocyclyl]₂—N—, H₂N—(C═O)—, HO—NH—(C═O)—, (C₁-C₆)alkyl-O—NH—(C═O)—, [(C₁-C₆)alkyl]-NH—(C═O)—, [(C₁-C₆)alkyl]₂—N—(C═O)—, [(C₃-C₇)cycloalkyl]-N H—(C═O)—, [(C₃-C₇)cycloalkyl]₂—N—(C═O)—, [(C₆-C₁₀)aryl]-N H—(C═O)—, [(C₆-C₁₀aryl]₂—N—(C═O)—, [(C₁-C₆)alkyl]-[((C₆-C₁₀)aryl)-N]-(C═O)—, [(C₁-C₉)heteroaryl]-NH—(C═O)—, [(C₁-C₉)heferoaryl]₂—N—(O═O)—, [(C₁-C₉)heterocyclyl]-NH—(C═O)—, (C₁-C₆)alkyl-S— and (C₁-C₆)alkyl optionally substituted by one —OH substituent or by one to four fluoro substituents; R²⁵³ is a saturated (3- to 4-membered)-heterocyclyl ring radical; or a saturated, partially saturated or aromatic (7- to 9-membered)-heterocyclyl ring radical;
wherein said saturated (3- to 4-membered)-heterocyclyl ring radical or said saturated, partially saturated or aromatic (7- to 9-membered)-heterocyclyl ring radical; may optionally contain one to four ring heteroatoms independently selected from the groups consisting of —N═, —NH—, —O—, and —S—;
wherein said saturated (3- to 4-membered)-heterooyclyl ring radical; or said saturated, partially saturated or aromatic (7- to 9-nembered)-heterocyclyl ring radical; may optionally be substituted on any ring carbon atom by one to three substituents per ring independently selected from the group consisting of halo, —OH, —CN, —NO₂, (C₂-C₆)alkenyl, (C₂-C₆)alkynyl, (C₃-C₇)cycloalkyl, (C₆-C₁₀)aryl, (C₂-C₉)hetorocyclyl, (C₁-C₆)alkyl-O—, H—(C═O)—, (C₁-C₆)alkyl-(C═O)—, HO—(C═O)—, (C₁-C₆)alkyl-O—(C═O)—, —NH₂, (C₁-C₆)alkyl-NH—, [(C₁-C₆) alkyl]₂—N—, (C₃-C₇)cycloalkyl-NH—, (C₆-C₁₀)aryl-NH—, [(C₁-C₆)alkyl]-[((C₆-C₁₀)aryl)-N]—, (C₁-C₉)heteroaryl-NH—, H₂N—(C═O)-[(C₁-C₆)alkyl]-NH—(C═O)—, [(C₁-C₆)alkyl]₂—N—(C═O)—, [(C₆-C₁₀)aryl]-NH—(C═O)—, [(C₁-C₆)alkyl]-[((C₆-C₁₀)aryl)-N]—(C═O)—, (C₁-C₆)alkyl-O—NH—(C═O)—, (C₁-C₆)alkyl-(C═O)—HN—, (C₁-C₆)alkyl-(C═O)-[(C₁-C₆)alkyl-N]—, —SH, (C₁-C₆)alkyl-S—, (C₁-C₆)alkyl-(S═O)—, (C₁-C₆)alkyl-SO₂— and (C₁-C₆)alkyl optionally substituted with one to fourfluoro moieties;
wherein said saturated (3- to 4-membered)-heterocyclyl ring radical; or said saturated, partially saturated or aromatic (7- to 9-membered)-heterocyclyl ring radical; may also optionally be substituted on any ring nitrogen atom by one to three substituents per ring independently selected from the group consisting of (C₃-C₇)cyoloalkyl, (C₆-C₁₀)aryl, (C₂-C₉)heterocyclyl, H—(C═O)—, (C₁-C₆)alkyl-(C═O)—, (C₁-C₆)alkyl-O—(C═O)—, H₂N—(C═O)—, [(C₁-C₆)alkyl]-NH—(C═O)—, [(C₁-C₆)alkyl]₂—N—(C═O)—, [(C₆-C₁₀)aryl]-NH—(C═O)—, [(C₁-C₆)alkyl]-[((C₆-C₁₀)aryl)-N]-(C═O)—, (C₁-C₆)alkyl-O—NH—(C═O)—, and (C₁-C₆)alkyl optionally substituted with one to four fluoro moieties;
R²⁵⁴ is an (C₁-C₆)alkyl radical optionally substituted by one to four fluoro substituents; and
R²⁵⁵ is a radical selected from the group consisting of H, halo, —OH, (C₁-C₆)alkyl-O—, (C₂-C₆)alkenyl, (C₂-C₆) alkynyl, (C₃-C₇)cycloalkyl, —CN, H—(C═O)—, (C₁-C₆)alkyl-(C═O)—, (C₁-C₆)alkyl-(C═O)—O—, HO—(C═O)—, (C₁-C₆)alkyl-O—(C═O)—, (C₁-C₆)alkyl-NH—. [(C₁-C₆)alkyl]₂—N—, (C₃-C₇)cycloalkyl-NH—, (C₆-C₁₀)aryl-NH—, [(C₁-C₆)alkyl]-[((C₆-C₁₀)aryl)-N]—, (C₁-C₉)heteroaryl-NH—, H₂N—(C═O)—, (C₁-C₆)alkyl-NH—(C═O)—. [(C₁-C₆)alkyl]₂—N—(C═O)—, (C₆-C₁₀)aryl-(C═O)—, [(C₁-C₆)alkyl]-[((C₆-C₁₀)aryl)-N]-(C═O)—, (C₁-C₆)alkyl-O—NH—(C═O)—, (C₁-C₆)alkyl-S—, and (C₁-C₆)alkyl optionally substituted by one to four fluoro substituents.

2-phenylpyran-4-one derivatives such as those described in U.S. Pat. No. 6,518,303 are also useful as Cox-2 selective inhibitors of the present invention. Such 2-phenylpyran-4-one derivatives have the general formula shown below in formula LIII: 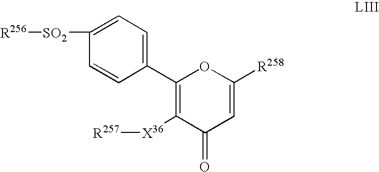
wherein:
R²⁵⁶ represents an alkyl or —NR²⁵⁹R²⁶⁰ group, wherein R²⁵⁹ and
R²⁶⁰ each independently represents a hydrogen atom or an alkyl group;
R²⁵⁷ represents an alkyl, C₃-C₇ cycloalkyl, naphthyl, tetrahydronaphthyl or indanyl group, or a phenyl group which may be unsubstituted or substituted by one or more halogen atoms or alkyl, trifluoromethyl, hydroxy, alkoxy, methylthio, amino, mono- or dialkylamino, hydroxyalkyl or hydroxycarbonyl groups;
R²⁵⁸ represents a methyl, hydroxymethyl, alkoxymethyl, C₃-C₇ cycloalkoxymethyl, benzyloxymethyl, hydroxycarbonyl, nitrile, trifluoromethyl or difluoromethyl group or a CH₂—R²⁶¹ group wherein R²⁶¹ represents an alkyl group; and X³⁶ represents a single bond, an oxygen atom, a sulfur atom or a methylene group;
   or a pharmaceutically acceptable salt thereof.

Examples of 2-phenylpyran-4-one derivatives useful in the present invention include, but are not limited to:
3-(4-fluorophenyl)-2-(4-methanesulfonylphenyl)-6-methylpyran-4-one,
3-(2-fluorophenyl)-2-(4-methanesulfonylphenyl)-6-methylpyran-4-one,
3-(4-chlorophenyl)-2-(4-methanesulfonylphenyl)-6-methylpyran-4-one,
3-(4-bromophenyl)-2-(4-methylsulfonylphenyl)-6-methylpyran-4-one,
3-(2,4-difluorophenyl)-2-(4-methanesulfonylphenyl)-6-methylpyran-4-one,
3-(3,4-dichlorophenyl)-2-(4-methanesulfonylphenyl)-6-methylpyran-4-one,
3-(3-chloro-4-methylphenyl)-2-(4-methanesulfonylphenyl)-6-methylpyran-4-one,
2-(4-methanesulfonylphenyl)-6-methyl-3-phenoxypyran-4-one,
3-(4-fluorophenoxy)-2-(4-methanesulfonylphenyl)-6-methylpyran-4-one,
3-(2-fluorophenoxy)-2-(methanesulfonylphenyl)-6-methylpyran-4-one,
3-(4-chlorophenoxy)-2-(methanesulfonylphenyl)-6-methylpyran-4-one,
3-(2-chlorophenoxy)-2-(methanesulfonylphenyl)-6-methylpyran-4-one,
3-(4-bromophenoxy)-2-(4-methanesulfonylphenyl)-6-methylpyran-4-one,
2-(4-methanesulfonylphenyl)-6-methyl-3-(4-methylphenoxy)pyran-4-one,
3-(2,4-difluorophenoxy)-2-(4-methanesulfonylphenyl)-6-methylpyran-4-one,
3-(2,5-difluorophenoxy)-2-(methanesulfonylphenyl)-6-methylpyran-4-one,
3-(4-chlorophenyl)-2-(4-methanesulfonylphenyl)-6-methoxymethylpyran-4-one,
3-(4-chlorophenyl)-6-difluoromethyl-2-(4-methanesulfonylphenyl)pyran-4-one,
   and pharmaceutically acceptable salts thereof.

Cox-2 selective inhibitors that are useful in the subject method and compositions can also include the compounds that are described in U.S. Pat. No. 6,472,416 (sulfonylphenylpyrazoles); U.S. Pat. No. 6,451,794 (2,3-diaryl-pyrazolo[1,5-b]pyridazines); U.S. Pat. Nos. 6,169,188, 6,020,343, and 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); U.S. Pat. Nos. 6,359,182 and 6,538,116 (C-nitroso compounds); U.S. Published Application No. 2003/0065011 (substituted pyridines); U.S. Published Application No. 2003/0207897 (substituted indole derivatives); and mixtures thereof.

Examples of specific compounds that are useful as Cox-2 selective inhibitors include, without limitation:
a1) 8-acetyl-3-(4-fluorophenyl)-2-(4-methylsulfonyl)phenyl-imidazo(1,2-a)pyridine;
a2) 5,5-dimethyl-4-(4-methylsulfonyl)phenyl-3-phenyl-2-(5H)-furanone;
a3) 5-(4-fluorophenyl)-1-[4-(methylsulfonyl)phenyl]-3-(trifluoromethyl)pyrazole;
a4) 4-(4-fluorophenyl)-5-[4-(methylsulfonyl)phenyl]-1-phenyl-3-(trifluoromethyl)pyrazole;
a5) 4-(5-(4-chlorophenyl)-3-(4-methoxyphenyl)-1H-pyrazol-1-yl)benzenesulfonamide
a6) 4-(3,5-bis(4-methylphenyl)-1H-pyrazol-1-yl)benzenesulfonamide;
a7) 4-(5-(4-chlorophenyl)-3-phenyl-1H-pyrazol-1-yl)benzenesulfonamide;
a8) 4-(3,5-bis(4-methoxyphenyl)-1H-pyrazol-1-yl)benzenesulfonamide;
a9) 4-(5-(4-chlorophenyl)-3-(4-methylphenyl)-1H-pyrazol-1-yl)benzenesulfonamide;
a10) 4-(5-(4-chlorophenyl)-3-(4-nitrophenyl)-1H-pyrazol-1-yl)benzenesulfonamide;
b1) 4-(5-(4-chlorophenyl)-3-(5-chloro-2-thienyl)-1H-pyrazol-1-yl)benzenesulfonamide;
b2) 4-(4-chloro-3,5-diphenyl-1H-pyrazol-1-yl)benzenesulfonamide b3) 4-[5-(4-chlorophenyl)-3-(trifluoromethyl)-1H-pyrazol-1-yl]benzenesulfonamide;
b4) 4-[5-phenyl-3-(trifluoromethyl)-1H-pyrazol-1-yl]benzenesulfonamide;
b5) 4-[5-(4-fluorophenyl)-3-(trifluoromethyl)-1H-pyrazol-1-yl]benzenesulfonamide;
b6) 4-[5-(4-methoxyphenyl)-3-(trifluoromethyl)-1H-pyrazol-1-yl]benzenesulfonamide;
b7) 4-[5-(4-chlorophenyl)-3-(difluoromethyl)-1H-pyrazol-1-yl]benzenesulfonamide;
b8) 4-[5-(4-methylphenyl)-3-(trifluoromethyl)-1H-pyrazol-1-yl]benzenesulfonamide;
b9) 4-[4-chloro-5-(4-chlorophenyl)-3-(trifluoromethyl)-1H-pyrazol-1-yl]benzenesulfonamide;
b10) 4-[3-(difluoromethyl)-5-(4-methylphenyl)-1H-pyrazol-1-yl]benzenesulfonamide;
c1) 4-[3-(difluoromethyl)-5-phenyl-1H-pyrazol-1-yl]benzenesulfonamide;
c2) 4-[3-(difluoromethyl)-5-(4-methoxyphenyl)-1H-pyrazol-1-yl]benzenesulfonamide;
c3) 4-[3-cyano-5-(4-fluorophenyl)-1H-pyrazol-1-yl]benzenesulfonamide;
c4) 4-[3-(difluoromethyl)-5-(3-fluoro-4-methoxyphenyl)-1H-pyrazol-1-yl]benzenesulfonamide;
c5) 4-[5-(3-fluoro-4-methoxyphenyl)-3-(trifluoromethyl)-1H-pyrazol-1-yl]benzenesulfonamide;
c6) 4-[4-chloro-5-phenyl-1H-pyrazol-1-yl]benzenesulfonamide;
c7) 4-[5-(4-chlorophenyl)-3-(hydroxymethyl)-1H-pyrazol-1-yl]benzenesulfonamide;
c8) 4-[5-(4-(N,N-dimethylamino)phenyl)-3-(trifluoromethyl)-1H-pyrazol-1-yl]benzenesulfonamide;
c9) 5-(4-fluorophenyl)-6-[4-(methylsulfonyl)phenyl]spiro[2.4]hept-5-ene;
c10) 4-[6-(4-fluorophenyl)spiro[2.4]hept-5-en-5-yl]benzenesulfonamide;
d1) 6-(4-fluorophenyl)-7-[4-(methylsulfonyl)phenyl]spiro[3.4]oct-6-ene;
d2) 5-(3-chloro-4-methoxyphenyl)-6-[4-(methylsulfonyl)phenyl]spiro[2.4]hept-5-ene;
d3) 4-[6-(3-chloro-4-methoxyphenyl)spiro[2.4]hept-5-en-5-yl]benzenesulfonamide;
d4) 5-(3,5-dichloro-4-methoxyphenyl)-6-[4-(methylsulfonyl)phenyl]spiro[2.4]hept-5-ene;
d5) 5-(3-chloro-4-fluorophenyl)-6-[4-(methylsulfonyl)phenyl]spiro[2.4]hept-5-ene;
d6) 4-[6-(3,4-dichlorophenyl)spiro[2.4]hept-5-en-5-yl]benzenesulfonamide;
d7) 2-(3-chloro-4-fluorophenyl)-4-(4-fluorophenyl)-5-(4-methylsulfonylphenyl)thiazole;
d8) 2-(2-chlorophenyl)-4-(4-fluorophenyl)-5-(4-methylsulfonylphenyl)thiazole;
d9) 5-(4-fluorophenyl)-4-(4-methylsulfonylphenyl)-2-methylthiazole;
d10) 4-(4-fluorophenyl)-5-(4-methylsulfonylphenyl)-2-trifluoromethylthiazole;
e1) 4-(4-fluorophenyl)-5-(4-methylsulfonylphenyl)-2-(2-thienyl)thiazole;
e2) 4-(4-fluorophenyl)-5-(4-methylsulfonylphenyl)-2-benzylaminothiazole;
e3) 4-(4-fluorophenyl)-5-(4-methylsulfonylphenyl)-2-(1-propylamino)thiazole;
e4) 2-[(3,5-dichlorophenoxy)methyl)-4-(4-fluorophenyl)-5-]4-(methylsulfonyl)phenyl]thiazole;
e5) 5-(4-fluorophenyl)-4-(4-methylsulfonylphenyl)-2-trifluoromethylthiazole;
e6) 1-methylsulfonyl-4-[1,1-dimethyl-4-(4-fluorophenyl)cyclopenta-2,4-dien-3-yl]benzene;
e7) 4-[4-(4-fluorophenyl)-1,1-dimethylcyclopenta-2,4-dien-3-yl]benzenesulfonamide;
e8) 5-(4-fluorophenyl)-6-[4-(methylsulfonyl)phenyl]spiro[2.4]hepta-4,6-diene;
e9) 4-[6-(4-fluorophenyl)spiro[2.4]hepta-4,6-dien-5-yl]benzenesulfonamide;
e10) 6-(4-fluorophenyl)-2-methoxy-5-[4-(methylsulfonyl)phenyl]-pyridine-3-carbon itrile;
f1) 2-bromo-6-(4-fluorophenyl)-5-[4-(methylsulfonyl)phenyl]-pyridine-3-carbonitrile;
f2) 6-(4-fluorophenyl)-5-[4-(methylsulfonyl)phenyl]-2-phenyl-pyridine-3-carbonitrile;
f3) 4-[2-(4-methylpyridin-2-yl)-4-(trifluoromethyl)-1H-imidazol-1-yl]benzenesulfonamide;
f4) 4-[2-(5-methylpyridin-3-yl)-4-(trifluoromethyl)-1H-imidazol-1-yl]benzenesulfonamide;
f5) 4-[2-(2-methylpyridin-3-yl)-4-(trifluoromethyl)-1H-imidazol-1-yl]benzenesulfonamide;
f6) 3-[1-[4-(methylsulfonyl)phenyl]-4-(trifluoromethyl)-1H-imidazol-2-yl]pyridine;
f7) 2-[1-[4-(methylsulfonyl)phenyl-4-(trifluoromethyl)-1H-imidazol-2-yl]pyridine;
f8) 2-methyl-4-[1-[4-(methylsulfonyl)phenyl-4-(trifluoromethyl)-1H-imidazol-2-yl]pyridine;
f9) 2-methyl-6-[1-[4-(methylsulfonyl)phenyl-4-(trifluoromethyl)-1H-imidazol-2-yl]pyridine;
f10) 4-[2-(6-methylpyridin-3-yl)-4-(trifluoromethyl)-1H-imidazol-1-yl]benzenesulfonamide;
g1) 2-(3,4-difluorophenyl)-1-[4-(methylsulfonyl)phenyl]-4-(trifluoromethyl)-1H-imidazole;
g2) 4-[2-(4-methylphenyl)-4-(trifluoromethyl)-1H-imidazol-1-yl]benzenesulfonamide;
g3) 2-(4-chlorophenyl)-1-[4-(methylsulfonyl)phenyl]-4-methyl-1H-imidazole;
g4) 2-(4-chlorophenyl)-1-[4-(methylsulfonyl)phenyl]-4-phenyl-1H-imidazole;
g5) 2-(4-chlorophenyl)-4-(4-fluorophenyl)-1-[4-(methylsulfonyl)phenyl]-1H-imidazole;
g6) 2-(3-fluoro-4-methoxyphenyl)-1-[4-(methylsulfonyl)phenyl-4-(trifluoromethyl)-1H-imidazole;
g7) 1-[4-(methylsulfonyl)phenyl]-2-phenyl-4-trifluoromethyl-1H-imidazole;
g8) 2-(4-methylphenyl)-1-[4-(methylsulfonyl)phenyl]-4-trifluoromethyl-1H-imidazole;
g9) 4-[2-(3-chloro-4-methylphenyl)-4-(trifluoromethyl)-1H-imidazol-1-yl]benzenesulfonamide;
g10) 2-(3-fluoro-5-methylphenyl)-1-[4-(methylsulfonyl)phenyl]-4-(trifluoromethyl)-1H-imidazole;
h1) 4-[2-(3-fluoro-5-methylphenyl)-4-(trifluoromethyl)-1H-imidazol-1-yl]benzenesulfonamide;
h2) 2-(3-methylphenyl)-1-[4-(methylsulfonyl)phenyl]-4-trifluoromethyl-1H-imidazole;
h3) 4-[2-(3-methylphenyl)-4-trifluoromethyl-1H-imidazol-1-yl]benzenesulfonamide;
h4) 1-[4-(methylsulfonyl)phenyl]-2-(3-chlorophenyl)-4-trifluoromethyl-1H-imidazole;
h5) 4-[2-(3-chlorophenyl)-4-trifluoromethyl-1H-imidazol-1-yl]benzenesulfonamide;
h6) 4-[2-phenyl-4-trifluoromethyl-1H-imidazol-1-yl]benzenesulfonamide;
h7) 4-[2-(4-methoxy-3-chlorophenyl)-4-trifluoromethyl-1H-imidazol-1-yl]benzenesulfonamide;
h8) 1-allyl-4-(4-fluorophenyl)-3-[4-(methylsulfonyl)phenyl]-5-(trifluoromethyl)-1H-pyrazole;
h9) 4-[1-ethyl-4-(4-fluorophenyl)-5-(trifluoromethyl)-1H-pyrazol-3-yl]benzenesulfonamide;
i1) N-phenyl-[4-(4-luorophenyl)-3-[4-(methylsulfonyl)phenyl]-5-(trifluoromethyl)-1H-pyrazol-1-yl]acetamide;
i2) ethyl [4-(4-fluorophenyl)-3-[4-(methylsulfonyl)phenyl]-5-(trifluoromethyl)-1H-pyrazol-1-yl]acetate;
i3) 4-(4-fluorophenyl)-3-[4-(methylsulfonyl)phenyl]-1-(2-phenylethyl)-1H-pyrazole;
i4) 4-(4-fluorophenyl)-3-[4-(methylsulfonyl)phenyl]-1-(2-phenylethyl)-5-(trifluoromethyl)pyrazole;
i5) 1-ethyl-4-(4-fluorophenyl)-3-[4-(methylsulfonyl)phenyl]-5-(trifluoromethyl)-1H-pyrazole;
i6) 5-(4-fluorophenyl)-4-(4-methylsulfonylphenyl)-2-trifluoromethyl-1H-imidazole;
i7) 4-[4-(methylsulfonyl)phenyl]-5-(2-thiophenyl)-2-(trifluoromethyl)-1H-imidazole;
i8) 5-(4-fluorophenyl)-2-methoxy-4-[4-(methylsulfonyl) phenyl]-6-(trifluoromethyl)pyridine;
i9) 2-ethoxy-5-(4-fluorophenyl)-4-[4-(methylsulfonyl)phenyl]-6-(trifluoromethyl)pyridine;
i10) 5-(4-fluorophenyl)-4-[4-(methylsulfonyl)phenyl]-2-(2-propynyloxy)-6-(trifluoromethyl)pyridine;
j1) 2-bromo-5-(4-fluorophenyl)-4-[4-(methylsulfonyl)phenyl]-6-(trifluoromethyl)pyridine;
j2) 4-[2-(3-chloro-4-methoxyphenyl)-4,5-difluorophenyl]benzenesulfonamide;
j3) 1-(4-fluorophenyl)-2-[4-(methylsulfonyl)phenyl]benzene;
j4) 5-difluoromethyl-4-(4-methylsulfonylphenyl)-3-phenylisoxazole;
j5) 4-[3-ethyl-5-phenylisoxazol-4-yl]benzenesulfonamide;
j6) 4-[5-difluoromethyl-3-phenylisoxazol-4-yl]benzenesulfonamide;
j7) 4-[5-hydroxymethyl-3-phenylisoxazol-4-yl]benzenesulfonamide;
j8) 4-[5-methyl-3-phenyl-isoxazol-4-yl]benzenesulfonamide;
j9) 1-[2-(4-fluorophenyl)cyclopenten-1-yl]-4-(methylsulfonyl)benzene;
j10) 1-[2-(4-fluoro-2-methylphenyl)cyclopenten-1-yl]-4-(methylsulfonyl)benzene;
k1) 1-[2-(4-chlorophenyl)cyclopenten-1-yl]-4-(methylsulfonyl)benzene;
k2) 1-[2-(2,4-dichlorophenyl)cyclopenten-1-yl]-4-(methylsulfonyl)benzene;
k3) 1-[2-(4-trifluoromethylphenyl)cyclopenten-1-yl]-4-(methylsulfonyl)benzene;
k4) 1-[2-(4-methylthiophenyl)cyclopenten-1-yl]-4-(methylsulfonyl)benzene;
k5) 1-[2-(4-fluorophenyl)-4,4-dimethylcyclopenten-1-yl]-4-(methylsulfonyl)benzene;
k6) 4-[2-(4-fluorophenyl)-4,4-dimethylcyclopenten-1-yl]benzenesulfonamide;
k7) 1-[2-(4-chlorophenyl)-4,4-dimethylcyclopenten-1-yl]-4-(methylsulfonyl)benzene;
k8) 4-[2-(4-chlorophenyl)-4,4-dimethylcyclopenten-1-yl]benzenesulfonamide;
k9) 4-[2-(4-fluorophenyl)cyclopenten-1-yl]benzenesulfonamide;
k10) 4-[2-(4-chlorophenyl)cyclopenten-1-yl]benzenesulfonamide;
l1) 1-[2-(4-methoxyphenyl)cyclopenten-1-yl]-4-(methylsulfonyl)benzene;
l2) 1-[2-(2,3-difluorophenyl)cyclopenten-1-yl]-4-(methylsulfonyl)benzene;
l3) 4-[2-(3-fluoro-4-methoxyphenyl)cyclopenten-1-yl]benzenesulfonamide;
l4) 1-[2-(3-chloro-4-methoxyphenyl)cyclopenten-1-yl]-4-(methylsulfonyl)benzene;
l5) 4-[2-(3-chloro-4-fluorophenyl)cyclopenten-1-30 yl]benzenesulfonamide;
l6) 4-[2-(2-methylpyridin-5-yl)cyclopenten-1-yl]benzenesulfonamide;
l7) ethyl 2-[4-(4-fluorophenyl)-5-[4-(methylsulfonyl)phenyl]oxazol-2-yl]-2-benzyl-acetate;
l8) 2-[4-(4-fluorophenyl)-5-[4-(methylsulfonyl)phenyl]oxazol-2-yl]acetic acid;
l9) 2-(tert-butyl)-4-(4-fluorophenyl)-5-[4-(methylsulfonyl)phenyl]oxazole;
l10) 4-(4-fluorophenyl)-5-[4-(methylsulfonyl)phenyl]-2-phenyloxazole;
m1) 4-(4-fluorophenyl)-2-methyl-5-[4-(methylsulfonyl)phenyl]oxazole; and
m2) 4-[5-(3-fluoro-4-methoxyphenyl)-2-trifluoromethyl-4-oxazolyl]benzenesulfonamide.
m3) 6-chloro-2-trifluoromethyl-2H-1-benzopyran-3-carboxylic acid;
m4) 6-chloro-7-methyl-2-trifluoromethyl-2H-1-benzopyran-3-carboxylic acid;
m5) 8-(1-methylethyl)-2-trifluoromethyl-2H-1-benzopyran-3-carboxylic acid;
m6) 6-chloro-7-(1,1-dimethylethyl)-2-trifluoromethyl-2H-1-benzopyran-3-carboxylic acid;
m7) 6-chloro-8-(1-methylethyl)-2-trifluoromethyl-2H-1-benzopyran-3-carboxylic acid;
m8) 2-trifluoromethyl-3H-naphthopyran-3-carboxylic acid;
m9) 7-(1,1-dimethylethyl)-2-trifluoromethyl-2H-1-benzopyran-3-carboxylic acid;
m10) 6-bromo-2-trifluoromethyl-2H-1-benzopyran-3-carboxylic acid;
n1) 8-chloro-2-trifluoromethyl-2H-1-benzopyran-3-carboxylic acid;
n2) 6-trifluoromethoxy-2-trifluoromethyl-2H-1-benzopyran-3-carboxylic acid;
n3) 5,7-dichloro-2-trifluoromethyl-2H-1-benzopyran-3-carboxylic acid;
n4) 8-phenyl-2-trifluoromethyl-2H-1-benzopyran-3-carboxylic acid;
n5) 7,8-dimethyl-2-trifluoromethyl-2H-1-benzopyran-3-carboxylic acid;
n6) 6,8-bis(dimethylethyl)-2-trifluoromethyl-2H-1-benzopyran-3-carboxylic acid;
n7) 7-(1-methylethyl)-2-trifluoromethyl-2H-1-benzopyran-3-carboxylic acid;
n8) 7-phenyl-2-trifluoromethyl-2H-1-benzopyran-3-carboxylic acid;
n9) 6-chloro-7-ethyl-2-trifluoromethyl-2H-1-benzopyran-3-carboxylic acid;
n10) 6-chloro-8-ethyl-2-trifluoromethyl-2H-1-benzopyran-3-carboxylic acid;
o1) 6-chloro-7-phenyl-2-trifluoromethyl-2H-1-benzopyran-3-carboxylic acid;
o2) 6,7-dichloro-2-trifluoromethyl-2H-1-benzopyran-3-carboxylic acid;
o3) 6,8-dichloro-2-trifluoromethyl-2H-1-benzopyran-3-carboxylic acid;
o4) 2-trifluoromethyl-3H-naptho[2,1-b]pyran-3-carboxylic acid;
o5) 6-chloro-8-methyl-2-trifluoromethyl-2H-1-benzopyran-3-carboxylic acid;
o6) 8-chloro-6-methyl-2-trifluoromethyl-2H-1-benzopyran-3-carboxylic acid;
o7) 8-chloro-6-methoxy-2-trifluoromethyl-2H-1-benzopyran-3-carboxylic acid;
o8) 6-bromo-8-chloro-2-trifluoromethyl-2H-1-benzopyran-3-carboxylic acid;
o9) 8-bromo-6-fluoro-2-trifluoromethyl-2H-1-benzopyran-3-carboxylic acid;
o10) 8-bromo-6-methyl-2-trifluoromethyl-2H-1-benzopyran-3-carboxylic acid;
p1) 8-bromo-5-fluoro-2-trifluoromethyl-2H-1-benzopyran-3-carboxylic acid;
p2) 6-chloro-8-fluoro-2-trifluoromethyl-2H-1-benzopyran-3-carboxylic acid;
p3) 6-bromo-8-methoxy-2-trifluoromethyl-2H-1-benzopyran-3-carboxylic acid;
p4) 6-[[(phenylmethyl)amino]sulfonyl]-2-trifluoromethyl-2H-1-benzopyran-3-carboxylic acid;
p5) 6-[(dimethylamino)sulfonyl]-2-trifluoromethyl-2H-1-benzopyran-3-carboxylic acid;
p6) 6-[(methylamino)sulfonyl]-2-trifluoromethyl-2H-1-benzopyran-3-carboxylic acid;
p7) 6-[(4-morpholino)sulfonyl]-2-trifluoromethyl-2H-1-benzopyran-3-carboxylic acid;
p8) 6-[(1,1-dimethylethyl)aminosulfonyl]-2-trifluoromethyl-2H-1-benzopyran-3-carboxylic acid;
P9) 6-[(2-methylpropyl)aminosulfonyl]-2-trifluoromethyl-2H-1-benzopyran-3-carboxylic acid;
p10) 6-methylsulfonyl-2-trifluoromethyl-2H-1-benzopyran-3-carboxylic acid;
q1) 8-chloro-6-[[(phenylmethyl)amino]sulfonyl]-2-trifluoromethyl-2H-1-benzopyran-3-carboxylic acid;
q2) 6-phenylacetyl-2-trifluoromethyl-2H-1-benzopyran-3-carboxylic acid;
q3) 6,8-dibromo-2-trifluoromethyl-2H-1-benzopyran-3-carboxylic acid;
q4) 8-chloro-5,6-dimethyl-2-trifluoromethyl-2H-1-benzopyran-3-carboxylic acid;
q5) 6,8-dichloro-(S)-2-trifluoromethyl-2H-1-benzopyran-3-carboxylic acid;
q6) 6-benzylsulfonyl-2-trifluoromethyl-2H-1-benzopyran-3-carboxylic acid;
q7) 6-[[N-(2-furylmethyl)amino]sulfonyl]-2-trifluoromethyl-2H-1-benzopyran-3-carboxylic acid;
q8) 6-[[N-(2-phenylethyl)amino]sulfonyl]-2-trifluoromethyl-2H-1-benzopyran-3-carboxylic acid;
q9) 6-iodo-2-trifluoromethyl-2H-1-benzopyran-3-carboxylic acid;
q10) 7-(1,1-dimethylethyl)-2-pentafluoroethyl-2H-1-benzopyran-3-carboxylic acid;
r1) 5,5-dimethyl-3-(3-fluorophenyl)-4-(4-methyl-sulphonyl-2(5H)-fluranone;
r2) 6-chloro-2-trifluoromethyl-2H-1-benzothiopyran-3-carboxylic acid;
r3) 4-[5-(4-chlorophenyl)-3-(trifluoromethyl)-1H-pyrazol-1-yl]benzenesulfonamide;
r4) 4-[5-(4-methylphenyl)-3-(trifluoromethyl)-1H-pyrazol-1-yl]benzenesulfonamide;
r5) 4-[5-(3-fluoro-4-methoxyphenyl)-3-(difluoromethyl)-1H-pyrazol-1-yl]benzenesulfonamide;
r6) 3-[1-[4-(methylsulfonyl)phenyl]-4-trifluoromethyl-1H-imidazol-2-yl]pyridine;
r7) 2-methyl-5-[1-[4-(methylsulfonyl)phenyl]-4-trifluoromethyl-1H-imidazol-2-yl]pyridine;
r8) 4-[2-(5-methylpyridin-3-yl)-4-(trifluoromethyl)-1H-imidazol-1-yl]benzenesulfonamide;
r9) 4-[5-methyl-3-phenylisoxazol-4-yl]benzenesulfonamide;
r10) 4-[5-hydroxymethyl-3-phenylisoxazol-4-yl]benzenesulfonamide;
s1) [2-trifluoromethyl-5-(3,4-difluorophenyl)-4-oxazolyl]benzenesulfonamide;
s2) 4-[2-methyl-4-phenyl-5-oxazolyl]benzenesulfonamide; or
s3) 4-[5-(3-fluoro-4-methoxyphenyl-2-trifluoromethyl)-4-oxazolyl]benzenesulfonamide;
   or a pharmaceutically acceptable salt or prodrug thereof.

Cox-2 inhibitors that are useful in the methods and compositions of present invention can be supplied by any source as long as the Cox-2 inhibitor is pharmaceutically acceptable. Likewise, Cox-2 inhibitors that are useful in the compositions and methods of present invention can be synthesized, for example, according to the description in Example 1. Several Cox-2 inhibitors that are suitable for use with the compositions and methods of the present invention may be synthesized by the methods described in, for example, in U.S. Pat. No. 5,466,823 to Talley, et al.

Preferred Cox-2 selective inhibitor compounds are those compounds selected from the group consisting of celecoxib, parecoxib, deracoxib, valdecoxib, etoricoxib, meloxicam, rofecoxib, lumiracoxib, RS 57067, T-614, BMS-347070 (Bristol Meyers Squibb, described in U.S. Pat. No. 6,180,651), JTE-522 (Japan Tabacco), S-2474 (Shionogi), SVT-2016, CT-3 (Atlantic Pharmaceutical), ABT-963 (Abbott), SC-58125 (GD Searle), nimesulide, flosulide, NS-398 (Taisho Pharmaceutical), L-745337 (Merck), RWJ-63556, L-784512 (Merck), darbufelone (Pfizer), CS-502 (Sankyo), LAS-34475 (Almirall Prodesfarma), LAS-34555 (Almirall Prodesfarma), S-33516 (Servier), SD-8381 (Pharmacia, described in U.S. Pat. No. 60,340,256), MK-966 (Merck), L-783003 (Merck), T-614 (Toyama), D-1376 (Chiroscience), L-748731 (Merck), CGP-28238 (Novartis), BF-389 (Biofor/Scherer), GR-253035 (Glaxo Wellcome), prodrugs of any of them, and mixtures thereof.

More preferred is that the Cox-2 selective inhibitor is selected from the group consisting of celecoxib, parecoxib, deracoxib, valdecoxib, lumiracoxib, etoricoxib, rofecoxib, prodrugs of any of them, and mixtures thereof.

Even more preferred still is that the Cox-2 selective inhibitor is celecoxib.

Cox-2 inhibitors that are useful in the methods and compositions and methods of present invention can be supplied by any source as long as the Cox-2 inhibitor is pharmaceutically acceptable.

Various classes of Cox-2 inhibitors useful in the present invention can be prepared as follows. Pyrazoles can be prepared by methods described in WO 95/15316. Pyrazoles can further be prepared by methods described in WO 95/15315. Pyrazoles can also be prepared by methods described in WO 96/03385.

Thiophene analogs useful in the present invention can be prepared by methods described in WO 95/00501. Preparation of thiophene analogs is also described in WO 94/15932.

Oxazoles useful in the present invention can be prepared by the methods described in WO 95/00501. Preparation of oxazoles is also described in WO 94/27980.

Isoxazoles useful in the present invention can be prepared by the methods described in WO 96/25405.

Imidazoles useful in the present invention, can be prepared by the methods described in WO 96/03388. Preparation of imidazoles is also described in WO 96/03387.

Cyclopentene Cox-2 inhibitors useful in the present invention can be prepared by the methods described in U.S. Pat. No. 5,344,991. Preparation of cyclopentene Cox-2 inhibitors is also described in WO 95/00501.

Terphenyl compounds useful in the present invention can be prepared by the methods described in WO 96/16934.

Thiazole compounds useful in the present invention can be prepared by the methods described in WO 96/03,392.

Pyridine compounds useful in the present invention can be prepared by the methods described in WO 96/03392. Preparation of pyridine compounds is also described in WO 96/24,585.

Benzopyranopyrazolyl compounds useful in the present invention can be prepared by the methods described in WO 96/09304.

Chromene compounds useful in the present invention can be prepared by the methods described in WO 98/47890. Preparation of chromene compounds is also described in WO 00/23433. Chromene compounds can further be prepared by the methods described in U.S. Pat. No. 6,077,850. Preparation of chromene compounds is further described in U.S. Pat. No. 6,034,256.

Arylpyridazinones useful in the present invention can be prepared by the methods described in WO 00/24719. Preparation of arylpyridazinones is also described in WO 99/10332. Arylpyridazinones can further be prepared by the methods described in WO 99/10331.

5-Alkyl-2-arylaminophenylacetic acids and derivatives useful in the present invention can be prepared by the methods described in WO 99/11605.

Diarylmethylidenefuran derivative Cox-2 selective inhibitors useful in the present invention can be prepared by the methods described in U.S. Pat. No. 6,180,651.

The celecoxib used in the compositions and methods of the present invention can be prepared in the manner set forth in U.S. Pat. No. 5,466,823.

The valdecoxib used in the compositions and methods of the present invention can be prepared in the manner set forth in U.S. Pat. No. 5,633,272.

The parecoxib used in the compositions and methods of the present invention can be prepared in the manner set forth in U.S. Pat. No. 5,932,598.

The rofecoxib used in the compositions and methods of the present invention can be prepared in the manner set forth in U.S. Pat. No. 5,474,995.

The deracoxib used in the compositions and methods of the present invention can be prepared in the manner set forth in U.S. Pat. No. 5,521,207.

The etoricoxib used in the compositions and methods of the present invention can be prepared in the manner set forth in WO 98/03484.

The meloxicam used in the compositions and methods of the present invention can be prepared in the manner set forth in U.S. Pat. No. 4,233,299.

The compound 4-(4-cyclohexyl-2-methyloxazol-5-yl)-2-fluorobenzenesulfonamide used in the compositions and methods of the present invention can be prepared in the manner set forth in U.S. Pat. No. 5,994,381.

The compound 2-(3,4-difluorophenyl)-4-(3-hydroxy-3-methylbutoxy)-5-[4-(methylsulfonyl)phenyl]-3(2H)-pyridazinone used in the compositions and methods of the present invention can be prepared in the manner set forth in WO 00/24719.

The compound 2-(3,5-difluorophenyl)-3-[4-(methylsulfonyl)phenyl]-2-cyclopenten-1-one used in the compositions and methods of the present invention can be prepared in the manner set forth in EP 863134.

The compound 2-[(2-chloro-6-fluorophenyl)amino]-5-methyl-benzeneacetic acid used in the compositions and methods of the present invention can be prepared in the manner set forth in WO 99/11605.

The compound N-[2-(cyclohexyloxy)-4-nitrophenyl]methanesulfonamide used in the compositions and methods of the present invention can be prepared in the manner set forth in U.S. Pat. No. 4,885,367.

The compound (3Z)-3-[(4-chlorophenyl)[4-(methylsulfonyl)phenyl]methylene]dihydro-2(3H)-furanone used in the compositions and methods of the present invention can be prepared in the manner set forth in U.S. Pat. No. 6,180,651.

Cox-2 inhibitors can also be isolated and purified from natural sources. Cox-2 inhibitors should be of a quality and purity that is conventional in the trade for use in pharmaceutical products.

A second component of the present invention is a muscarinic receptor antagonist that is administered in combination with a Cox-2 inhibitor to a subject.

As used herein, the phrase “muscarinic receptor antagonist” means an agent or compound, or a combination of two or more of such agents or compounds, which inhibits the activity of a muscarinic receptor, including any of the subtypes of muscarinic receptors. Currently, muscarinic receptors exhibit 5 different subtypes, namely M₁, M₂, M₃, M₄ and M₅.

The present invention encompasses the use of muscarinic receptor antagonists, including non-selective muscarinic receptor antagonists. In preferred embodiments, the muscarinic receptor antagonist is a selective muscarinic receptor antagonist.

For purposes of the present invention, the use of the terms “non-selective muscarinic receptor antagonist” refers to a muscarinic receptor antagonist that is capable of inhibiting, to varying degrees, all of the muscarinic subtypes. The use of the terms “selective muscarinic receptor antagonist” refers to a muscarininc receptor antagonist that is capable of inhibiting or antagonizing the activity of one or more muscarinic receptor subtypes to a greater degree than other muscarinic receptor subtypes.

For example, the present invention encompasses muscarinic receptor antagonists that inhibit the muscarinic receptor subtypes M₁, M₂, M₃, M₄, and M₅, or is capable of blocking the action of acetylcholine at a muscarinic receptor. However, it has been found that the method and compositions of the present invention are particularly effective when the muscarinic receptor antagonist is selective for the inhibition of the muscarinic receptor subtype M₁ or the subtype M₃, or both.

In one embodiment, tiotropium bromide or (Spiriva®), in particular, has been found to be a preferred muscarinic receptor antagonist that is selective for the muscarinic receptor subtypes M₁ and M₃.

In still another embodiment, the present invention encompasses the muscarinic receptor antagonists described in Table 3 below.

TABLE 3
Muscarinic Receptor Antagonists

| | | Trade | Drug | | | |
|---|---|---|---|---|---|---|
| No. | Compound Name | Name(s) | Class | Dose | Manufacturer | Reference |
| A1 | Tiotropium Bromide | Spiriva ® | M₁ and M₃ | Once | Boehringer | Littner, M., et al., Long-acting |
| | (1α, 2β, 4β, 5α, 7β)-7- | | selective | daily | Ingelheim | bronchodilation with once- |
| | [(hydroxy-2- | | muscarinic | inhaler | Pharmaceuticals, | daily dosing of tiotropium |
| | thienylacetyl)oxy]-9,9- | | receptor antagonist | | Inc. | (Spiriva) in stable chronic |
| | dimethyl-3-oxa-9- | | | | | obstructive pulmonary |
| | azoniatricyclo | | | | | disease, Am. J. Respir. Crit. |
| | [3.3.1.0.sup.2,4]nonane | | | | | Care Med.,161: 1136-1142 |
| | bromide monohydrate | | | | | (2000). |
| A2 | Quinuclidinyl benzilate | | Non-specific | | | Saunders, P.A., et al., |
| | quinuclidinyl-a- | | muscarinic | | | Antagonists have a greater |
| | hydroxydiphenylacetate | | receptor | | | selectivity for muscarinic |
| | | | antagonist | | | receptor subtypes in intact |
| | | | | | | cerebellar granule cells than |
| | | | | | | in membranes Brain Res. |
| | | | | | | 713: 29-35 (1996) |
| A3 | Butylscopolamine | Buscopan ® | Non-specific | 30-80 | Boehringer | Merck Index 12 1624. |
| | bromide | | muscarinic | mg | Ingelheim | Pfaffendorf, et al., |
| | [(7S)-(1a,2β,4β,5a,7β)[- | | receptor | daily | Pharmaceuticals, | Comparison of various |
| | 9-butyl-7-(3-hydroxy-1 - | | antagonist | | Inc. | spasmolytic drugs on guinea- |
| | oxo-2-phenylpropoxy)-9- | | | | | pig isolated common bile |
| | methyl-3-oxa-9- | | | | | duct. Naunyn-Schmied. Arch. |
| | azoniatricyclo[3.3.1.02,4] | | | | | Pharmacol. 344: 114 (1991). |
| | nonane bromide | | | | | |
| A4 | 4-DAMP | | M₃-selective | | | Barlow, et al., A further |
| | 1,1-dimethyl-4- | | muscarinic | | | search for selective |
| | diphenylacetoxypiperi- | | receptor | | | antagonists at M₂-muscarinic |
| | dinium iodide | | antagonist | | | receptors. Br. J. Pharmacol. |
| | | | | | | 89: 837 (1986). |
| A5 | Ipratropium bromide | Atrovent ® | Non-specific | 500 μg | Boehringer | Merck Index 12 5089. |
| | (endo,syn)-(±)-3-(3- | | muscarinic | 3-4 | Ingelheim | Fryer, et at., Ipratropium |
| | hydroxy-1-oxo-2- | | receptor | times a | Pharmaceuticals, | bromide potentiates |
| | phenylpropoxy)-8- | | antagonist; | day by | Inc. | bronchoconstriction induced |
| | methyl-8-(1- | | broncho- | oral | | by vagal nerve stimulation in |
| | methylethyl)-8- | | dilator. | nebulization | the guinea-pig. Eur. J. | |
| | azoniabicyclo | | | 6 to 8 | | Pharmacol. 139: 187 (1987). |
| | [3.2.1]octane bromide | | | hours apart. | | |
| A6 | Nitrocaramiphen | | M₁ selective | | | Hudkins, et at., Caramiphen, |
| | hydrochloride | | muscarinic | | | iodocaramiphen and |
| | 2-diethylaminoethyl 1-(4- | | receptor | | | nitrocaramiphen are potent, |
| | nitrophenyl) | | antagonist. | | | competitive, muscarine M₁ |
| | cyclopentanecarboxylate | | | | | receptor selective agents. |
| | | | | | | Eur. J. Pharmacol. 231: 485 |
| | | | | | | (1993). |
| A7 | Pirenzepine | | M₁ selective | | | Hammer, et al., Pirenzepine |
| | dihydrochloride | | muscarinic | | | distinguishes between |
| | 5,11-dihydro-11-[(4- | | receptor | | | different subclasses of |
| | methyl-1- | | antagonist. | | | muscarinic receptors. Nature |
| | piperazinyl)acetyl]-6H- | | | | 283: 90 (1980). | |
| | pyrido [2,3- | | | | | |
| | b][1,4]benzodiazepin-6- | | | | | |
| | one | | | | | |
| A8 | Scopolamine | | Non-specific | | | Parkes, An examination of |
| | hydrobromide | | muscarinic | | | central actions characteristic |
| | (hyoscine | | receptor | | | of scopolamine: comparison |
| | hydrobromide) | | antagonist | | | of central and peripheral |
| | α-(hydroxymethyl) | | | | | activity in scopolamine, |
| | benzeneacetic acid 9- | | | | | atropine and some synthetic |
| | methyl-3-oxa-9- | | | | | basic esters. |
| | azatricyclo [3.3.1.02,4] | | | | | Psychopharmacologia 7:1 |
| | non-7-yl ester | | | | | (1965). |
| | hydrobromide | | | | | |
| A9 | Telenzepine | | M₁ -selective | | | Galvan, et al., Interaction of |
| | dihydrochloride | | muscarinic | | | telenzepine with muscarinic |
| | 4,9-dihydro-3-methyl-4- | | receptor | | | receptors in mammalian |
| | [(4-methyl-1- | | antagonist | | | sympathetic ganglia. |
| | piperazinyl)acetyl]-10H- | | | | | Eur. J. Pharmacol. 167: 1 |
| | thieno[3,4- | | | | | (1989). |
| | b][1,5]benzodiazepin-10- | | | | | |
| | one Dihydrochioride | | | | | |
| A10 | Tropicamide | Mydriacyl ®*, | M₄ selective | | *Alcon | Lazareno, et al., Characterisation of |
| | N-Ethyl-3-hydroxy-2- | Opticyl ®, | muscarinic | | Laboratories, | muscarinic M₄ binding sites in |
| | phenyl-N- | Tropicacyl ®** | antagonist. | | Inc.; **Akorn, | rabbit lung, chicken heart and NG |
| | (pyridinylmethyl) | | | | Inc, | 108-15 cells. Mol. Pharmacol. |
| | propanamide | | | | | 38: 805 (1990). |
| | 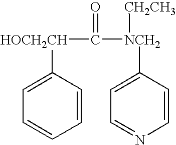 | | | | | |
| A11 | W-84 | | Stabilises | | | Mohr, et al., Equipotent |
| | Hexamethylene-bis- | | muscarinic | | | allosteric effect of W84 on |
| | [dimethyl-(3- | | antagonist- | | | [³H]-NMS binding to cardiac |
| | phthalimidopropyl) | | receptor | | | muscarinic receptors from |
| | ammonium] bromide | | complexes | | | guinea-pig, rat and pig. |
| | | | by an | | | Pharmacol. Toxicol. 70: 198 |
| | | | allosteric | | | (1992). |
| | | | effect. | | | |
| A12 | Atropine sulfate | Atropine | Non-specific | 2 mg | Abbott | Zwart, R., and Vijverberg, |
| | 1αH,5αH-tropan-3-α | sulfate | muscarinic | doses | Laboratories | H.P., Potentiation and inhibition |
| | ol (±)-tropate(ester), | injection | receptor | | | of neuronal nicotinic receptors by |
| | sulfate (2:1) | | antagonist | | | atropine: competitive and |
| | (salt) | | | | | noncompetitive effects Mol. |
| | monohydrate | | | | | Pharmacol. 52: 886-895 (1997). |
| | 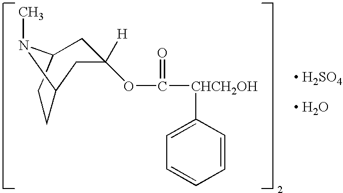 | | | | | |
| A13 | Glycopyrrolate | Robinul ® | Non-specific | 2-6 mgs | American | Haddad, F., et al., Pharmacological |
| | 3-[(cyclopentylhydroxyphenyl- | and | muscarinic | daily | Home Products | characteriztion of the muscarinic |
| | acetyl)oxy]-1,1-dimethylpyrrolidinium bromide | Robinul ® | receptor | | Corp. | receptor antagonis, glycopyrrolate, in |
| | | | | | | human and guinea-pig airways. Br. J. |
| | | | | | | Pharmacol. 127: 413-20 (1999). |
| A14 | Scopolamine | Transderm | Non-specific | 1 mg over 3 | Novartis | Clissold, etal., Trandsdermal hyoscine |
| | aα-(hydroxymethyl) benzeneacetic acid 9- | | muscarinic | days | | (scopolamine), a preliminary review of |
| | methyl-3-oxa-9-azatricyclo [3.3.1.02,4] | | receptor | | | its pharmacodynamic properties and |
| | non-7-yl ester | | | | | therapeutic efficacy. |
| | | | | | | drugs, 29: 189-207 (1985). |
| 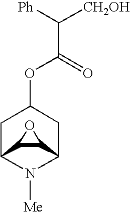 | | | | | | |
| A15 | Benztropine mesylate | Congentin ® | Non-specific | 1-2 mg | Merck & Co., | Goff, D.C., et al., The effect of |
| | 8-azabicyclo[3.2.1] | | muscarinic | daily; | Inc. | benztropine on haloperidol- |
| | octane, 3- | | receptor | up to 6 | | induced dystonia, clinical |
| | (diphenylmethoxy)-, endo, | | antagonist | mg daily | | efficacy and pharmacokinetics: a |
| | methanesulfonate | | | daily | | prospective, double-blind trial. |
| | | | | | | J Gli Psychopharmacol. |
| | | | | | | 11(2): 106-12(1991). |
| A16 | YM-905 (solifenacinsuccinate) | | M₃-selective | | | Kobayashi, S., et al., Effects |
| | (1S,3′R)-quinuclidin-3′-yl | | muscarinic | | | of YM905, a novel muscarinic |
| | 1-phenyl-1,2,3,4- | | receptor | | | M3-receptor antagonist, on |
| | tetrahydroisoquinoline-2- | | antagonist | | | experimental models of bowel |
| | carboxylate | | | | | dysfunction in vivo. Jpn J |
| | | | | | | Pharmacol, 86(3): 281-8 (2001). |
| A17 | Tripitramine | | M₂-selective | | | Roffel, A.F., et al., |
| | 1,1,24-tris[[5,11-dihydro- | | muscarinic | | | Characterization of the |
| | 6-oxo-6H-pyrido [2,3- | | receptor | | | muscarinic receptor |
| | b][1,4]-benzodiazepin- | | antagonist | | | subtype(s) mediating |
| | 11-yl)carbonyl]methyl]-8, | | | | | contraction of the guinea-pig |
| | 17-dimethyl-1,8,17,24- | | | | | lung strip and inhibition of |
| | tetraazatetracosane | | | | | acetylcholine release in the |
| | tetraoxalate | | | | | guinea-pig trachea with the |
| | | | | | | selective muscarinic receptor |
| | | | | | | antagonist tripitramine. Br. J. |
| | | | | | | Pharmacol., 122: 133-41 (1997). |
| A18 | Cyclopentolate | Cyclogyl ® | Non-specific | | Alcon | Ishikawa H., et al., Selectivity |
| | hydrochloride | Pentolair ® | muscarinic | | Laboratories, | of muscarinic agonists |
| | 2-(Dimethylamino)ethyl | AK- | receptor | | Inc. | including (+/−)-aceclidine and |
| | 1-hydroxy-a- | Pentolate ® | antagonist | | Bausch & | antimuscarinics on the human |
| | phenylcyclopentane | | | | Lomb | intraocular muscles. |
| | acetate hydrochloride | | | | Pharmaceuticals, | J Ocul Pharmacol Ther. |
| | | | | | Inc. | 14: 363-73 (1998). |
| | | | | | Akom, Inc. | |
| A19 | J-104129 | | M₃-selective | | | Mitsuya, M., et al., J-104129, |
| | (2R)-N-[1-(4-methyl-3- | | muscarinic | | | a novel muscarinic M₃ |
| | pentenyl)piperidin-4-yl]- | | receptor | | | receptor antagonist with high |
| | 2-cyclopentyl-2-hydroxy- | | antagonist | | | selectivity for M₃ over M₂ |
| | 2-phenylacetamide | | | | | receptors. Bioorg Med Chem, 7: |
| | | | | | | 2555-67 (1999). |
| A20 | (+/−)-Terodiline | | M₁-selective | | | Noronha-Blob, L., et al., (+/−)- |
| | N-tert-Butyl-1-methyl- | | muscarinic | | | Terodiline, an M₁-selective |
| | 3,3-diphenylpropylamine | | receptor | | | muscarinic receptor |
| | | | antagonist | | | antagonist. In vivo effects at |
| | | | | | | muscarinic receptors |
| | | | | | | mediating urinary bladder |
| | | | | | | contraction, mydriasis and |
| | | | | | | salivary secretion. Eur J |
| | | | | | | Pharmacol, 201: 135-42 (1991). |
| A21 | Methoctramine tetrahydrochloride | | M₂-selective | | | Watson, N., et al., Actions of |
| | N,N′-bis[6-[[(2- | | muscarinic | | | methoctramine, a muscarinic |
| | Methoxyphenyl)methyl]amino] | | receptor | | | M₂ receptor antagonist, on |
| | hexyl]-1,8-octane diamine | | | | | muscarinic and nicotinic |
| | tetrahydrochloride | | | | | cholinoceptors in guinea-pig |
| | | | | | | airways in vivo and in vitro. |
| | | | | | | Br. J. Pharmacol., 105: 107-12 (1992). |
| A22 | (2R)-2-[(1R)-3,3- | | M₃-selective | | | Mitsuya, M., et al., A potent, |
| | difluorocyclopentyl]-2- | | muscarinic | | | long-acting, orally active (2R)- |
| | hydroxy-2-phenylacetamide | | receptor | | | 2-[(1R)-3,3- |
| | | | antagonist | | | difluorocyclopentyl]-2- |
| | | | | | | hydroxy-2-phenylacetamide: |
| | | | | | | novel muscarinic M(3) |
| | | | | | | receptor antagonist with high |
| | | | | | | selectivity for M(3) over M(2) |
| | | | | | | receptors. J Med Chem. |
| | | | | | | 43: 5017-29 (2000). |
| A23 | Clozapine | Clozaril ®, | Non-specific | 300- | Novartis | Bolden, C., et al., Clozapine is |
| | 8-chloro-11-(4-methyl-1- | Leponex ® | muscarinic | 900 | Pharmaceuticals | a potent and selective |
| | piperazinyl)-5H-dibenzo | | receptor | mg/day | | muscarinic antagonist at the |
| | [b,e] [1,4] diazepine | | antagonist | | | five cloned human muscarinic |
| | | | | | | acetylcholine receptors |
| | | | | | | expressed in CHO-K1 cells. |
| | | | | | | Eur J Pharmacol, 192: 205-6 |
| | | | | | | (1991). |
| A24 | PNU-171990 | | Non-specific | | | Modiri, A.R., et al., |
| | 2-diisopropyl aminoethyl | | muscarinic | | | Characterization of a new |
| | 1-phenylcyclopentane | | receptor | | | muscarinic receptor |
| | carboxylate | | antagonist | | | antagonist PNU-171990 in |
| | hydrochloride | | | | | guinea pig, cat and human |
| | | | | | | smooth muscle. Eur J |
| | | | | | | Pharmacol, 451: 171-5 (2002). |
| A25 | Tolterodine tartrate | Detrol ® | Non-specific | 2-4 | Pharmacia & | Nilvebrant, L., Tolterodine and |
| | (R)-2-[3-[bis(1- | | muscarinic | mg/day | Upjohn Co. | its active 5-hydroxymethyl |
| | methylethyl)-amino]-1- | | receptor | | | metabolite: pure muscarinic |
| | phenylpropyl]-4- | | antagonist | | | receptor antagonists. |
| | methylphenol [R- | | | | | Pharmacol Toxicol, 90: 260-7 |
| | (R*,R*)]-2,3-dihydroxybutanedioate | | | | | (2002). |
| A26 | Cyclohexylmethyl- | | M₃ selective | | | Sagara, Y., et al., |
| | piperidinyltriphenyl- | | muscarinic | | | Cyclohexylmethylpiperidinyltri- |
| | propioamide | | receptor | | | phenylpropioamide: a |
| | | | antagonist | | | selective muscarinic M(3) |
| | | | | | | antagonist discriminating |
| | | | | | | against the other receptor |
| | | | | | | subtypes. J Med Chem, |
| | | | | | | 45: 984-7 (2002). |
| A27 | SCH 57790 | | M₂ selective | | | Carey, G. J., et al., SCH |
| | 4-cyclohexyl-alpha-[4-[[4- | | muscarinic | | | 57790, a selective muscarinic |
| | methoxyphenyl]sulfinyl]- | | receptor | | | M(2) receptor antagonist, |
| | phenyl]-1-piperazineacetonitrile | | | | | releases acetylcholine and |
| | | | | | | produces cognitive |
| | | | | | | enhancement in laboratory |
| | | | | | | animals. Eur J Pharmacol, |
| | | | | | | 431: 189-200 (2001). |
| A28 | Tiquizium bromide | | Non-specific | | | Shioya, T., et al., |
| | [3-(di-2- | | muscarinic | | | Antimuscarinic effect of |
| | thienylmethylene)-5 | | receptor | | | tiquizium bromide in vitro and |
| | methyl-trans- | | antagonist | | | in vivo. Fur J Clin Pharmacol, |
| | guinolizidinium bromide | | | | | 50: 375-80 (1996). |
| A29 | Oxitropium bromide | Oxivent ® | Non-specific | 400- | Boehringer | Flohr E., and Bischoff K.O., |
| | (8r)-6β,7β-epoxy-8-ethyl- | | muscarinic | 600 μg | Ingeiheim | Oxitropium bromide, a new |
| | 3a-hydroxy-1 aH,5aH- | | receptor | per day | International | anticholinergic drug, in a |
| | tropanium bromide (−)- | | antagonist | | | dose-response and placebo |
| | tropate | | | | | comparison in obstructive |
| | | | | | | airway diseases. Respiration. |
| | | | | | | 38: 98-104 (1979). |
| A30 | Pirenzepine Dihydrochloride | | M₁-selective | | | Lammers, J.W., et al., The |
| | 5,11-dihydro-11-[(4- | | muscarinic | | | role of pirenzepine-sensitive |
| | methyl-1- | | receptor | | | (M₁) muscarinic receptors in |
| | piperazinyl)acetyl]-6H- | | antagonist | | | vagally mediated |
| | pyrido[2,3- | | | | | bronchoconstriction in |
| | b][1,4]benzodiazepin-6- | | | | | humans. Am Rev Respir Dis. |
| | one dihydrochloride | | | | | 139: 446-9 (1989). |
| | 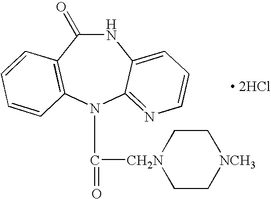 | | | | | |
| A31 | Revatropate | | M₁ and M₃- | | | Alabaster V.A., Discovery & |
| | (R)-3-quinuclidinyl (S)-β- | | selective | | | development of selective M₃ |
| | hydroxy-a-[2-(R)- | | muscarinic | | | antagonists for clinical use. |
| | methylsulfinyl]ethyl]hydrtropate | | receptor | | | Life Sd 60M₃-selective |
| | | | antagonist | | | muscarinic receptor |
| | | | | | | antagonist: 1053-60 (1997). |
| A32 | AQ-RA 721 | | M₁ and M₃- | | | Doods H.N., Selective |
| | | | selective | | | muscarinic antagonists as |
| | | | muscarinic | | | bronchodilators. Agents |
| | | | receptor | | | Actions Suppl 34: 117-30 |
| | | | antagonist | | | (1991). |
| A33 | DAC 5889 | | Muscarinic | | | Progress in Inflammation |
| | | | receptor | | | Research, (M. J. Parnham, |
| | | | antagonist | | | Ed.) 2001 Birkhäuser Verlag, |
| | | | | | | Basel (Switzerland) |
| | | | | | | Novel perspectives in |
| | | | | | | anticholinergic therapy |
| | | | | | | By Bernd Disse |
| A34 | Hyoscyamine sulfate | Levsin ® | Non-specific | 0.125- | Schwarz | Claussen, D.W. Levsin |
| | benzeneacetic acid | and | muscarinic | 0.5 mg | Pharma, Inc. | (hyoscyamine sulfate USP). |
| | α-(hydroxymethyl)-, 8- | Levsinex ® | receptor | 3-4 times | | Gastroenterol Nurs. 17: 37-8 |
| | methyl-8-azabicyclo | | antagonist | dialy and | | (1994). |
| | [3.2.1.] oct-3-yl ester, | | | 0.375-0.75 | | |
| | [3(S)-endo)-, sulfate | | | mg every | | |
| | (2:1),dihydrate | | | 12 hrs | | |
| A35 | Oxybutynin | Ditropan ®, | Non-specific | 10-20 | Ortho-McNeil | Staskin, D.R., Treatment of |
| | d, I (reacemic) 4- | Ditripan XL ® | muscarinic | mg/day | Pharmaceutical, | overactive bladder with once- |
| | diethylamino-2-butynyl | | receptor | | Inc. | daily extended-release |
| | phenylcyclohexylglycolate | | antagonist | | | tolterodine or oxybutynin: the |
| | | | | | | Antimuscarinic Clinical |
| | | | | | | Effectiveness Trial (ACET). |
| | | | | | | Curr Urol Rep. 3: 31-3 (2002). |
| A36 | Propantheline bromide | Pro- | Non-specific | 75 mg/day | Roberts | Yu, J.C. and Sung, R.J., |
| | 2-(Hydroxyethyl)diisopropyl | Banthine ® | muscarinic | | Laboratories, | Clinical efficacy of |
| | methylammonium bromide | | receptor | | Inc. | propantheline bromide in |
| | xanthene-9-carboxylate | | antagonist | | | neurocardiogenic syncope: |
| | | | | | | pharmacodynamic |
| | | | | | | implications. Cardiovasc |
| | | | | | | Drugs Ther. 10: 687-92 |
| | | | | | | (1997). |
| A37 | Flavoxate | Urispas ® | Non-specific | 300-800 | Smith-Kline | Uckert, S., et al., Responses |
| | 2-piperidinoethyl 3- | | muscarinic | mg/day | Beecham | of isolated normal human |
| | methyl-4-oxo-2-phenyl- | | receptor | | | detrusor muscle to various |
| | 4H-1-benzopyran-8- | | antagonist | | | spasmolytic drugs commonly |
| | carboxylate | | | | | used in the treatment of the |
| | | | | | | overactive bladder. |
| | | | | | | Arzneimittelforschung. 50: |
| | | | | | | 456-60 (2000). |
| A38 | Dicyclomine | Bentyl ® | M₁-selective | 80-160 | Merrell | Jiang, Z.W., etal., |
| | [bicyclohexyl-]1- | | muscarinic | mg/day | Pharmaceuticals, | Dicyclomine, an M₁ |
| | carboxylic acid, 2- | | receptor | | Inc. | muscarinic antagonist, |
| | (diethylammo) ethyl ester | | antagonist | | | reduces infarct volume in a rat |
| | | | | | | subdural hematoma model. |
| | | | | | | Brain Fes. 852: 37-44 2000. |
| A39 | PD 102807 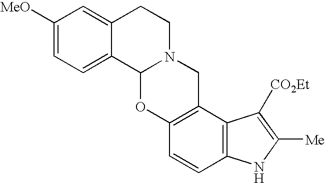 | | M₄ selective muscarinic receptor antagonist | | | Olianis, M.C. and Onali, P., PD 102807, a novel muscarinic M₄ receptor antagonist, discriminates between striatal and cortical muscarinic receptors coupled to cyclic AMP. Life Sci. 65: 2233-40 (1999). |
| A40 | Gallamine triethiodide | Flaxedil | M₂ selective | 1-2 mg/kg | | Gnagey, A.L., et al., Site-directed |
| | 2,2′,2-[1,2,3- | | muscarinic | body weight | | mutagenesis reveals two epitopes |
| | Benzenetriyltris(oxy)]tris | | receptor | | | involed in the subtype selectivity |
| | [N,N,N- | | antagonist | | | of the allosteric iteractions of |
| | triethylethanaminium]triiodide | | | | | gallamine at muscarinic |
| | 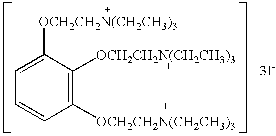 | | | | | acetylcholine receptors. Mol. Pharmacol. 56: 1245-53 (1999). |
| A41 | Himbacine | | M₂ and M₄ | | | Dorje, F., et al., Antagonist |
| | (3S,3αR,4R,4αS,8αR,9αS)- | | selective | | | binding profiles of five cloned |
| | 4-[(1E)-2-[(2R,6S)- | | muscarinic | | | human muscarinic receptor |
| | 1,6-dimethyl-2- | | receptor | | | subtypes J. Pharmacol. Exp. |
| | piperdinyl[ethenyl[decahydro- | | antagonist | | | Ther. 256: 727-33 (1991). |
| | 3-methyl-naphtho[2,3-c]furan-1(3H)-one | | | | | |
| A42 | AF-DX116/Otenzepad | | M₂ selective | | | Hammer, et al., Binding |
| | 11-([2- | | muscarinic | | | profile of a novel |
| | [(diethylamino)methyl]-1- | | receptor | | | cardioselective muscarinic |
| | piperdinyl]acetyl0-5,11- | | antagonist | | | receptor antagonist, AF-DX |
| | dihydro-6-pyrido[2,3- | | | | | 116, to membranes of |
| | b][1,4]benzodiazepin-6-one | | | | | peripheral tissues and brain in |
| | | | | | | the rat. Life Sd. 38: 1653 (1986). |
| A43 | AF-DX 384 | | M₂ and M₄ | | | Martin, J., et al., Syntheses of |
| | 5,11-dihydro-11-[2-[2- | | selective | | | R and S isomers of AF-DX |
| | [(N,N- | | muscarinic | | | 384, a selective antagonist of |
| | dipropylaminomethyl)piperidin-1- | | receptor | | | muscarinic M₂ receptors. |
| | yl]ethylamino]- | | antagonist | | | Bioorg Med Chem. 8: 591-600 |
| | carbonyl]6H-pyrido[2,3- | | | | | (2000). |
| | b[[1,4] benzodiazepin-6-one | | | | | |
| A44 | p-Fluorohexahydro- | | M₃ selective | | | Lambrecht, G., et al., Affinity |
| | sila-difenidol | | muscarinic | | | profiles of hexahydro-sila- |
| | hydrochloride | | receptor | | | difenidol analogues at |
| | Cyclohexyl-(4- | | antagonist | | | muscarinic receptor subtypes |
| | fluorophenyl)-(3-N- | | | | | Eur. J. Pharmacol. 168: 71-80 |
| | piperidinopropyl)silanol | | | | | (1989). |
| | hydrochloride | | | | | |
| A45 | Olanzapine | Zyprexa ®, | Non-specific | 10-15 | Eli Lilly and | Kennedy, J.S. et al., The |
| | 2-methyl-4-(4-methyl-1- | Lanzac ® | muscarinic | mg | Co. | central cholinergic system |
| | piperazinyl)-10H- | | receptor | daily | | profile of olanzapine |
| | thieno[2,3- | | antagonist | | | compared with placebo in |
| | b][1,5]benzodiazepine | | | | | Alzheimer's disease. Int J |
| | | | | | | Geriatr Psychiatry. Suppl |
| | | | | | | 1: S24-32 (2001). |
| A46 | Methscolpamine | Pamine | Non-specific | 12.5 mg | Bradley | Domino, E.F, et al., Central |
| | bromide | | muscarinic | daily | Pharmaceuticals, | and peripheral effects of |
| | 3-oxa-9-azoniatricyclo | | receptor | | Inc. | muscarinic cholinergic |
| | [3.3.1.0]nonane, 7-(3- | | antagonist | | | blocking agents in man. |
| | hydroxy-1-oxo-2- | | | | | Anesthesiology, 28: 568-574 |
| | phenylpropoxy)-9, 9- | | | | | (1967). |
| | dimethyl-, bromide, | | | | | |
| | [7(S)-(1α,2β,4β,5α,7β)] | | | | | |
| A47 | Trihexyphenidyl | Artane ® | Non-specific | 5-15 mg | American | Mavridis, M., et al., Chronic |
| | hydrochloride | | muscarinic | daily | Cyanamid | blockade of muscarinic |
| | 3-(1-piperidyl)-1-phenyl- | | receptor | | Company | cholinergic receptors by |
| | cyclohexyl-1-propanol | | antagonist | | | systemic trihexyphenidyl |
| | hydrochloride | | | | | (Artane) administration |
| | | | | | | modulates but does not |
| | | | | | | mediate the dopaminergic |
| | | | | | | regulation of striatal |
| | | | | | | prepropeptide messenger |
| | | | | | | RNA expression. |
| | | | | | | Neuroscience. 66: 37≅53 |
| | | | | | | (1995). |
| A48 | Darifenacin | Enablex ® | M₃ selective | N/A | Pfizer | Nunn P.A., et al., The binding |
| | (S)-1-[2-(2,3-Dihydro-5- | | muscarinic | | | profile of the novel muscarinic |
| | benzofuranyl)ethyl]-a,a- | | receptor | | | receptor antagonist |
| | diphenyl-3- | | antagonist | | | darifenacin against the five |
| | pyrrolidineacetamide | | | | | cloned human muscarinic |
| | | | | | | receptors expressed in CHO |
| | | | | | | cells. Br J Pharmacol |
| | | | | | | 117: 130P (1996). |
| A49 | Homatropine | Isopto | Non-specific | | Alcon | Cantor, E.H., et al., Structure |
| | hydrobromide | Homatropine ® | muscarinic | | Laboratories, | activity requirements for |
| | 3a-Hydroxy-1aH,5aH- | | receptor | | Inc. | hypotension and alpha- |
| | tropanium mandelate | | antagonist | | | adrenergic receptor blockade |
| | (ester) hydrobromide | | | | | by analogues of atropine. Eur |
| | | | | | | J Pharmacol. 90: 75-83 |
| | | | | | | (1983). |
| A50 | Lu 25-109 | | M₂ and M₃- | | | Waldeck K., et al., Actions of |
| | 2-ethyl-5-(1-methyl- | | selective | | | the new antimuscarinic |
| | 1,2,5,6- | | muscarinic | | | compound Lu 25-109 on |
| | tetrahydropyridyl)-2H- | | receptor | | | isolated human and pig |
| | tetrazole maleate | | antagonist; | | | detrusor. Neurourol Urodyn |
| | | M₁-selective | | | 21: 92-8 (2002). | |
| | | muscarinic | | | | |
| | | receptor | | | | |
| | | agonist | | | | |
| A51 | Fesoterodine | | Non-specific | | Schwarz | Bayes M., et al., Gateways to |
| | 2-[(1R)-3- | | muscarinic | | Pharma | Clinical Trials. Methods Find |
| | (Diisopropylamino)-1- | | receptor | | | Exp Clin Pharmacol, 24: 703- |
| | phenylpropyl]-4- | | agonist | | | 29 (2002). |
| | (hydroxymethyl)phenyl | | | | | |
| | isobutyrate | | | | | |
| A52 | 1,3-dihydro-1-{1- | | Non-specific | | | U.S. Pat. No. 5,691,323 |
| | [piperidin-4- | | muscarinic | | | |
| | yl]piperidin-4-yl}-2H- | | receptor | | | |
| | benzimidizol-2-ones | | agonist | | | |
| A53 | 1,3-dihydro-1-{4-amino- | | Non-specific | | | U.S. Pat. No. 5,691,323 |
| | 1-cyclohexyl}-2H- | | muscarinic | | | |
| | benzimidazol-2-ones | | receptor | | | |
| | | | agonist | | | |
| A54 | Benzocycloalkylenylamine | | M₂ and M₃- | | | U.S. Pat. No. 6,500,822 |
| | derivatives | | selective | | | |
| | | | muscarinic | | | |
| | | | receptor | | | |
| | | | antagonist | | | |
| A55 | (2R)-N-[1-(6- | | M₃-selective | | | Hirose,H., et al., |
| | aminopyridin-2- | | muscarinic | | | Pharmacological properties of |
| | ylmethyl)piperidin-4- | | receptor | | | (2R)-N-[1-(6-aminopyridin-2- |
| | yl]-2-[(1R)-3,3- | | antagonist | | | ylmethyl)piperidin-4-yl]-2- |
| | difluorocyclopentyl]-2- | | | | | [(1R)-3,3-difluorocyclopentyl]- |
| | hydroxy-2-phenylacetamide | | | | | 2-hydroxy-2- |
| | | | | | | phenylacetamide: a novel |
| | | | | | | muscarinic antagonist with |
| | | | | | | M₂-sparing antagonistic |
| | | | | | | sctivity. J. Pharmacol. Exp. |
| | | | | | | Ther., 297: 790-797(2001). |
| A56 | 2-[(1S,3S)-3- | | M₃-selective | | | Mitsuya M., et al., Discovery |
| | sulfonylaminocyclopentyl] | | muscarinic | | | of a muscarinic M₃ receptor |
| | phenylacetamide derivatives | | receptor | | | antagonist with high |
| | | | antagonist | | | selectivity for M₃ over M₂ |
| | | | | | | receptors among 2-[(1S,3S)-3- |
| | | | | | | sulfonylaminocyclopentyl] |
| | | | | | | phenylacetamide derivatives. |
| | | | | | | Bioorg Med Chem, 8: 825-32 |
| | | | | | | 2000. |
| A57 | Zamifenacin | | M₃-selective | | | Houghton L.A., et al., |
| | (R)-3- | | muscarinic | | | Zamifenacin (UK-76, 654) a |
| | (Diphenylmethoxy)-1 - | | receptor | | | potent gut M₃ selective |
| | [3,4-(methylenedioxy) | | antagonist | | | muscarinic antagonist, |
| | phenethyl]piperidine | | | | | reduces colonic motor activity |
| | | | | | | in patients with irritable bowel |
| | | | | | | syndrome. Ailment Pharmacol |
| | | | | | | Ther 11: 561-8 (1997). |
| A58 | (2S,3′R) 3- | | M₃-selective | | | Ghelardini C., et al., In vitro |
| | quinuclidinyl tropate | | muscarinic | | | characterization of a novel, |
| | | | receptor | | | potent and selective M₃ |
| | | | antagonist | | | antagonist. Life Sci 61: 1217- |
| | | | | | | 26 (1997). |
| A59 | 3-quinuclidinyl 3- | | M₃-selective | | | U.S. Pat. No. 5,543,419 |
| | hydroxymethyl 2- | | muscarinic | | | |
| | phenyl alkanoates | | receptor | | | |
| | | | antagonist | | | |
| A60 | 3-quinuclidinyl 3- | | M₃-selective | | | U.S. Pat. No. 5,543,419 |
| | hydroxymethyl 2- | | muscarinic | | | |
| | thienyl alkanoates | | receptor | | | |
| | | | antagonist | | | |
| A61 | 1-azabicyclo[2.2.2]octan- | | M₃-selective | | | U.S. Pat. No. 4,843,074 |
| | 3-yl 2-aryl-3-azacyclo- | | muscarinic | | | |
| | 2-hydroxypropionates | | receptor | | | |
| | | | antagonist | | | |
| A62 | 3-quinuclidinol esters | | M₁-selective | | | U.S. Pat. No. 4,644,033 |
| | | | muscarinic | | | |
| | | | receptor | | | |
| | | | antagonist | | | |
| A63 | unsymmetrical alpha- | | M₁-selective | | | U.S. Pat. No. 4,644,033 |
| | disubstituted glycolic | | muscarinic | | | |
| | acids | | receptor | | | |
| | | | antagonist | | | |
| A64 | KRP-197 | | M1 & M3 | | | |
| | 4-(2-methylimidazolyl)- | | receptor subtype | | | |
| | 2,2-diphenylbutyramide | | selective antagonist | | | |

The present invention encompasses the muscarinic receptor antagonists selected from the group consisting of tiotropium bromide, butylscopolamine bromide, quinuclidinyl benzilate quinuclidinyl-a-hydroxydiphenylacetate, 1,1-dimethyl-4-diphenylacetoxypiperidinium iodide, ipratropium bromide, nitrocaramiphen hydrochloride, pirenzepine dihydrochloride, scopolamine hydrobromide, telenzepine dihydrochloride, tropicamide, hexamethylene-bis-[dimethyl-(3-phthalimidopropyl) ammonium]bromide, atropine sulfate, glycopyrrolate, scopolamine, benztropine mesylate (1S, 3′R)-quinuclidin-3′-yl 1-phenyl-1, 2, 3, 4-tetrahydroisoquinoline-2-carboxylate, tripitramine, cyclopentolate hydrochloride, clozapine, (2R)-N-[1-(4-methyl-3-pentenyl)piperidin-4-yl]-2-cyclopentyl-2-hydroxy-2-phenylacetamide, (+/−)-terodiline, methoctramine tetrahydrochloride, (2R)-2-[(1R)-3,3-difluorocyclopentyl]-2-hydroxy-2-phenylacetamide, 2-diisopropyl aminoethyl 1-phenylcyclopentane carboxylate hydrochloride, tolterodine tartrate, cyclohexylmethyl-piperidinyltriphenyl-propioamide, 4-cyclohexyl-alpha-[4-[[4-methoxyphenyl]sulfinyl]-phenyl]-1-piperazine acetonitrile, tiquizium bromide, oxitropium bromide, pirenzepine dihydrochloride, revatropate, AQ-RA 721, DAC 5889, hyoscyamine sulfate, oxybutynin, propantheline bromide, flavoxate, dicyclomine, PD 102807, gallamine triethiodide, himbacine, otenzepad, 5,11-dihydro-11-[2-[2-[(N,N-dipropylaminomethyl)piperidin-1-yl]ethylamino]-carbonyl]6H-pyrido[2,3-B][1,4]benzodiazepin-6-one, pfluorohexahydro-sila-difenidol hydrochloride, cyclohexyl-(4-fluorophenyl)-(3-N-piperidinopropyl)silanol hydrochloride, olanzapine, methscolpamine bromide, trihexyphenidyl hydrochloride, darifenacin, homatropine hydrobromide, 2-ethyl-5-(1-methyl-1,2,5,6-tetrahydropyridyl)-2H-tetrazole maleate, 1,3-dihydro-1-{1-[piperidin-4-yl]piperidin-4-yl}-2H-benzimidizol-2-ones, 1,3-dihydro-1-{4-amino-1-cyclohexyl}-2H-benzimidazol-2-ones, benzocycloalkylenylamine derivatives, fesoterodine, (2R)-N-[1-(6-aminopyridin-2-ylmethyl)piperidin-4-yl]-2-[(1R)-3,3-difluorocyclopentyl]-2-hydroxy-2-phenylacetamide, 2-[(1S, 3S)-3-sulfonylaminocyclopentyl]phenylacetamide derivatives, zamifenacin, (2S, 3′R) 3-quinuclidinyl tropate, 3-quinuclidinyl 3-hydroxymethyl 2-phenyl alkanoates, 3-quinuclidinyl 3-hydroxymethyl 2-thienyl alkanoates, 1-azabicyclo[2.2.2]octan-3-yl 2-aryl-3-azacyclo-2-hydroxypropionates, 3-quinuclidinol esters, unsymmetrical alpha-disubstituted glycolic acids, benztropine, methscopolamine, KRP-197, tropicamide, and mixtures thereof.

Also encompassed by the present invention is a muscarinic receptor antagonist, tiotropium bromide (tiotropium), which selectively blocks M₁ and M₃ receptors. In patients with COPD, tiotropium has been shown to produce a dose-dependent and prolonged bronchodilator effect for as long as 48 hours. Tiotropium also produces prolonged bronchodilation in subjects suffering from asthma. Thus, also encompassed by the present invention is tiotropium, and any pharmaceutically acceptable salt thereof, including for example, the tiotropium salt, tiotropium bromide (1α, 2β, 4β, 5α, 7β)-7-[(hydroxy-2-thienylacetyl)oxy]-9,9-dimethyl-3-oxa-9-azoniatricyclo[3.3.1.0.sup.2,4]nonane bromide monohydrate].

In addition to classification based on selectivity for a particular muscarinic receptor subtype, the muscarinic receptor antagonists of the present invention can also be classified, in one embodiment, into additional categories. Thus, in another embodiment, the muscarinic receptor antagonists that are suitable for use with the present invention include those muscarinic receptor antagonists that are selected from the group consisting of 1) naturally-occurring compounds: including atropine, hyoscine (scopolamine) and homatropine (semi-synthetic); 2) tertiary amines: including dicyclomine, tropicamide, benztropine; and 3) quarternary amines: propantheline, glycopyrrolate, ipratropium.

Any combination of the Cox-2 inhibitors and muscarininc receptor antagonists that are described above can be used in novel compositions, pharmaceutical compositions and kits of the present invention. For example, a Cox-2 inhibitor such as celecoxib can be combined with any of the aforementioned muscarinic receptor antagonists described in table 3, including the muscarinic receptor antagonist, tiotropium bromide or any other muscarinic receptor antagonists, including glycopyrronium bromide and esters of bi- and tricyclic amino alcohols, are suitable for use with the present invention, such as are known from European disclosure document 0 418 716 and International Patent Application WO 92/16528.

In one embodiment, the present invention encompasses a novel therapuetic composition comprising a Cox-2 inhibitor and a muscarinic receptor antagonist.

In the present invention, a composition comprising a Cox-2 inhibitor in combination with a muscarinic receptor antagonist is administered to a subject according to standard routes of drug delivery that are well known to one of ordinary skill in the art.

Each of the Cox-2 inhibitors and muscarinic receptor antagonists of the present invention can be supplied in the form of a salt, or prodrug, if desirable. Cox-2 inhibitors and muscarinic receptor antagonists that are useful in the present invention can be of any purity or grade, as long as the preparation is of a quality suitable for pharmaceutical use. The Cox-2 inhibitors and muscarinic receptor antagonists can be provided in pure form, or it can be accompanied with impurities or commonly associated compounds that do not affect its physiological activity or safety.

The Cox-2 inhibitors and muscarinic receptor antagonists can be supplied in the form of a pharmaceutically active salt, a prodrug, an isomer, a tautomer, a racemic mixture, or in any other chemical form or combination that, under physiological conditions, still provides for inhibition of the Cox-2 enzyme and any physiological function that the muscarinic receptor antagonist may perform. The present invention includes all possible diastereomers as well as their racemic and resolved, enantiomerically pure forms.

The compounds useful in the present invention can have no asymmetric carbon atoms, or, alternatively, the useful compounds can have one or more asymmetric carbon atoms. When the useful compounds have one or more asymmetric carbon atoms, they, therefore, include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention. Also included in the methods, combinations and compositions of the present invention are the tautomeric forms of the described compounds.

Also included in the methods and compositions of the present invention are the prodrugs of the described compounds and the pharmaceutically acceptable salts thereof. The term “prodrug” refers to drug precursor compounds which, following administration to a subject and subsequent absorption, are converted to an active species in vivo via some process, such as a metabolic process. Other products from the conversion process are easily disposed of by the body. More preferred prodrugs produce products from the conversion process that are generally accepted as safe. A nonlimiting example of a “prodrug” that will be useful in the methods, combinations and compositions of the present invention is parecoxib (N-[[4-(5-methyl-3-phenyl-4-isoxazolyl)phenyl]sulfonyl]propanamide).

The term “pharmaceutically acceptable” is used adjectivally herein to mean that the modified noun is appropriate for use in a pharmaceutical product.

The compounds of the present invention can also be supplied in the form of a pharmaceutically acceptable salt. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids.

Exemplary pharmaceutically acceptable salts include the salts of hydrochloric acid and trifluoroacetic acid. All of the above salts can be prepared by those skilled in the art by conventional means from the corresponding compound of the present invention.

In another embodiment of the present invention, the combination of a Cox-2 inhibitor and a muscarinic receptor antagonist can be provided in a “pharmaceutically acceptable carrier” or “pharmaceutically acceptable excipient”, both of which are used interchangeably herein, to form a pharmaceutical composition. Thus, in one embodiment, the present invention encompasses a pharmaceutical composition comprising a Cox-2 inhibitor, a muscarinic receptor antagonist, and a pharmaceutically acceptable carrier.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. The pharmaceutically acceptable carrier can also be selected on the basis of the desired route of administration of the compound. For example, in a preferred embodiment the carrier is suitable for oral administration.

The carrier should be acceptable in the sense of being compatible with the other ingredients of the composition and not be deleterious to the recipient. The carrier can be a solid or a liquid, or both, and is preferably formulated with the compound as a unit-dose composition, for example, a tablet, which can contain from 0.05% to 95% by weight of the active compound.

Other pharmacologically active substances can also be present, including other compounds of the present invention. The pharmaceutical compositions of the invention can be prepared by any of the well-known techniques of pharmacy, such as by admixing the components.

The Cox-2 inhibitors or the muscarinic receptor antagonists can be administered by any conventional means available for use in conjunction with pharmaceuticals, either as individual therapeutic compounds or as a combination of therapeutic compounds or as a single pharmaceutical composition or as independent multiple pharmaceutical compositions.

Pharmaceutical compositions according to the present invention include those suitable for oral, inhalation spray, rectal, topical, buccal (e.g., sublingual), or parenteral (e.g., subcutaneous, intramuscular, intravenous, intrathecal, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

The compositions of the present invention can be administered enterally, by inhalation spray, rectally, topically, buccally or parenterally in dosage unit formulations containing conventional nontoxic pharmaceutically acceptable carriers, adjuvants, and vehicles as desired. Parenteral administration includes subcutaneous, intramuscular, intradermal, intramammary, intravenous, and other administrative methods known in the art. Enteral administration includes solution, tablets, sustained release capsules, enteric-coated capsules, and syrups. When administered, the pharmaceutical composition may be at or near body temperature.

In combination therapy, administration of two or more of the therapeutic agents useful in the methods and compositions of the present invention may take place sequentially in separate formulations, or may be accomplished by simultaneous administration in a single formulation or in a separate formulation. The formulation may be in the form of a bolus, or in the form of aqueous or non-aqueous isotonic sterile injection solutions or suspensions. For example, the therapeutic compounds which make up the combination therapy may be a combined dosage form or in separate dosage forms intended for substantially simultaneous oral administration. The therapeutic compounds, which make up the combination therapy, may also be administered sequentially, with either therapeutic compound being administered by a regimen calling for two-step ingestion. Thus, a regimen may call for sequential administration of the therapeutic compounds with spaced-apart ingestion of the separate, active agents. The time period between the multiple ingestion steps may range from, for example, a few minutes to several hours to days depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of any of the pharmaceutically acceptable excipients, diluents or other formulations components described herein. Thus, the combination of therapeutic compounds may be administered by any combination of, for example, oral/oral, oral/parenteral, or parenteral/parenteral route.

The compounds of the present invention can be delivered orally either in a solid, in a semi-solid, or in a liquid form. Oral (intra-gastric) is a preferred route of administration. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin. Solutions and suspensions may be prepared from sterile powders or granules having one or more pharmaceutically acceptable carriers or diluents, or a binder such as gelatin or hydroxypropylmethyl cellulose, together with one or more of a lubricant, preservative, surface active or dispersing agent.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of a muscarinic receptor antagonist, the dose range may be from about 0.01 mg to about 5,000 mg or any other dose, dependent upon the specific modulator, as is known in the art. When in a liquid or in a semi-solid form, the combinations of the present invention can, for example, be in the form of a liquid, syrup, or contained in a gel capsule (e.g., a gel cap). In one embodiment, when a muscarinic receptor antagonist is used in a combination of the present invention, the muscarinic receptor antagonist can be provided in the form of a liquid, syrup, or contained in a gel capsule. In another embodiment, when a Cox-2 inhibitor is used in a combination of the present invention, the Cox-2 inhibitor can be provided in the form of a liquid, syrup, or contained in a gel capsule.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Syrups and elixirs containing the Cox-2 inhibitor and muscarinic receptor antagonist may be formulated with sweetening agents, for example glycerol, sorbitol, or sucrose. Such formulations may also contain a demulcent, a preservative and flavoring and coloring agents. Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

The subject method of prescribing a Cox-2 inhibitor and muscarinic receptor antagonist and compositions comprising the same can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents, which have been mentioned above or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these therapeutic compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Administration of either one or both of the Cox-2 inhibitor and muscarinic receptor antagonist can also be by inhalation, in the form of aerosols or solutions for nebulizers. Therefore, in one embodiment, the Cox-2 inhibitor and muscarinic receptor antagonist are administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Aerosols of liquid particles comprising the active materials may be produced by any suitable means, such as inhalatory delivery systems. Nebulizers are commercially available devices, which transform solutions, or suspensions of the active ingredient into a therapeutic aerosol mist by means of acceleration of compressed gas, typically either air or oxygen, through a narrow venturi orifice or by means of ultrasonic agitation. Suitable formulations for use in nebulizers consist of the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles comprising the active materials may likewise be produced with any solid particulate medicament aerosol generator. Aerosol generators for administering solid particulate medicaments to a subject produce particles, which are respirable, as explained above, and generate a volume of aerosol containing a predetermined metered dose of a medicament at a rate suitable for human administration.

One type of solid particulate aerosol generator is an insufflator. Suitable formulations for administration by insufflation include finely comminuted powders, which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by means of air drawn through the device upon inhalation or by means of a manually operated pump. The powder employed in the insufflator either consists solely of the active ingredient or of a powder blend comprising the active materials, a suitable powder diluent, such as lactose, and an optional surfactant.

A second type of aerosol generator is a metered dose inhaler. Metered dose inhalers are pressurized aerosol dispensers, typically containing a suspension or solution formulation of the Cox-2 inhibitor and the muscarinic receptor antagonist in a liquefied propellant. During use, the metered dose inhaler discharges the formulation through a valve, adapted to deliver a metered volume, to produce a fine particle spray containing the active materials. Any propellant may be used for aerosol delivery, including both chlorofluorocarbon-containing propellants and non-chlorofluorocarbon-containing propellants.

A third type of aerosol generator is a electrohydrodynamic (EHD) aerosol generating device, which has the advantage of being adjustable to create substantially monomodal aerosols having particles more uniform in size than aerosols generated by other devices or methods. Typical EHD devices include a spray nozzle in fluid communication with a source of liquid to be aerosolized, at least one discharge electrode, a first voltage source for maintaining the spray nozzle at a negative (or positive) potential relative to the potential of the discharge electrode, and a second voltage source for maintaining the discharge electrode at a positive (or negative) potential relative to the potential of the spray nozzle. Most EHD devices create aerosols by causing a liquid to form droplets that enter a region of high electric field strength. The electric field then imparts a net electric charge to these droplets, and this net electric charge tends to remain on the surface of the droplet. The repelling force of the charge on the surface of the droplet balances against the surface tension of the liquid in the droplet, thereby causing the droplet to form a cone-like structure known as a Taylor Cone. In the tip of this cone-like structure, the electric force exerted on the surface of the droplet overcomes the surface tension of the liquid, thereby generating a stream of liquid that disperses into a many smaller droplets of roughly the same size. These smaller droplets form a mist, which constitutes the aerosol cloud that the user ultimately inhales.

Administration of the compositions of the present invention can also be rectally. Pharmaceutical compositions suitable for rectal administration are preferably presented as unit-dose suppositories. These can be prepared by admixing a compound or compounds of the present invention with one or more suitable non-irritating excipients, for example, cocoa butter, synthetic mono- di- or triglycerides, fatty acids and polyethylene glycols that are solid at ordinary temperatures, but liquid at the rectal temperature and will therefore melt in the rectum and release the drug; and then shaping the resulting mixture.

Administration may also be by transvaginal delivery through the use of an intravaginal device. Transvaginal delivery may be desirable for many certain subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Pharmaceutical compositions suitable for topical application to the skin preferably take the form of an ointments, creams, lotions, pastes, gels, sprays, powders, jellies, collyriums, solutions or suspensions, aerosols, or oils. Carriers, which can be used, include petroleum jelly (e.g., Vaseline®), lanolin, polyethylene glycols, alcohols, and combinations of two or more thereof. The active compound or compounds are generally present at a concentration of from 0.1 to 50% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research 3(6):318 (1986).

The compositions of the present invention can optionally be supplemented with additional agents such as, for example, viscosity enhancers, preservatives, surfactants and penetration enhancers.

Viscosity is an important attribute of many medications. Drops that have a high viscosity tend to stay in the body for longer periods and thus, increase absorption of the active compounds by the target tissues or increase the retention time. Such viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives are optionally employed to prevent microbial contamination during use. Suitable preservatives include polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g., Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a Cox-2 inhibitor and muscarinic receptor antagonist topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

Other methods for administration of the Cox-2 inhibitor compound and the muscarinic receptor antagonist include dermal patches that release the medicaments directly into a subject's skin.

Topical delivery systems are also encompassed by the present invention and include ointments, powders, sprays, creams, jellies, collyriums, solutions or suspensions.

Powders have the advantage of sticking to moist surfaces, and consequently, can remain on the skin for long periods. Therefore, powders are especially attractive for certain purulent respiratory disorders.

Pharmaceutically acceptable excipients and carriers encompass all the foregoing and the like. The above considerations concerning effective formulations and administration procedures are well known in the art and are described in standard textbooks. See e.g., Gennaro, A. R., Remington: The Science and Practice of Pharmacy, 20^{th} Edition, (Lippincott, Williams and Wilkins), (2000); Hoover, John E., Remington's Pharmaceutical Sciences, Mack Publishing Co., Easton Pa., (1975); Liberman, et al., Eds., Pharmaceutical Dosage Forms, Marcel Decker, New York, N.Y., (1980); and Kibbe, et al., Eds., Handbook of Pharmaceutical Excipients (3^{rd} Ed.), American Pharmaceutical Association, Washington, (1999).

Preferably, the present methods and compositions comprise a combination therapy, which can be used for preventing or treating a respiratory disorder, such as asthma and COPD, in a subject that is in need of the prevention or treatment of this type of disease or disorder.

For purposes of the present invention, it is preferred that the amount of a Cox-2 inhibitor and the amount of a muscarinic receptor antagonist comprise an effective amount of each of the two treatment agents. Further preferred is that the amount of the combination therapy with the Cox-2 inhibitor and muscarinic receptor antagonist together comprises a therapeutically effective amount of the combined therapy.

As used herein, an “effective amount” means the dose or amount to be administered to a subject and the frequency of administration to the subject, which is readily determined by one having ordinary skill in the art, by the use of known techniques and by observing results obtained under analogous circumstances.

In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances.

As used herein, the terms “therapeutically effective” are intended to qualify the amount of an agent for use in therapy that will achieve the goal of preventing or improving the severity of the disorder being treated, while avoiding adverse side effects typically associated with alternative therapies. A respiratory disorder symptom or a respiratory disorder-related complication symptom is considered ameliorated or improved if any benefit is achieved, no matter how slight.

For example, any reduction in inflammation, bronchospasm, bronchoconstriction, shortness of breath, wheezing, lower extremity edema, ascites, productive cough, hemoptysis, or cyanosis in a subject suffering from a respiratory disorder such as COPD, no matter how slight, would be considered an ameliorated symptom.

As used herein, the terms “prophylactically effective” refer to an amount of a Cox-2 inhibitor in combination with a muscarinic receptor antagonist that causes a decrease in the frequency of incidence of respiratory disorders or respiratory disorder-related complication. The term “prophylactic” refers to the prevention of respiratory disorders or a respiratory disorder-related complication, whereas the term “therapeutic” refers to the effective treatment of an existing disorder such as respiratory disorders or a respiratory disorder-related complication.

It will be appreciated that the amount of the Cox-2 inhibitor and the muscarinic receptor antagonist required for use in the treatment or prevention of respiratory disorders and respiratory disorder-related complications will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages.

The appropriate dosage level of a Cox-2 inhibitor will generally be from about 0.01 mg per kg to about 140 mg per kg subject body weight per day, which may be administered in single or multiple doses. Preferably, the dosage level will be about 0.1 mg/kg to about 25 mg/kg per day; more preferably about 0.5 mg/kg to about 10 mg/kg per day.

In larger mammals, for example humans, a typical indicated dose is about 0.5 mg to 7 grams orally per day. A compound may be administered on a regimen of several times per day, for example 1 to 4 times per day, preferably once or twice per day.

The amount of the Cox-2 inhibitor that may be combined with the carrier materials to produce a single dosage form will vary depending upon the host treated and the particular mode of administration. For example, a formulation intended for the oral administration of humans may contain from 0.5 mg to 7 g of active agent compounded optionally with an appropriate and convenient amount of carrier material, which may vary from about 5 to about 95 percent of the total composition. Dosage unit forms for the Cox-2 inhibitor will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

The dosage level of a muscarinic receptor antagonist will necessarily depend on the particular muscarinic receptor antagonist that is used. However, in general, the appropriate dosage level of a muscarinic receptor antagonist will generally be from about 0.0001 mg per kg to about 200 mg per kg subject body weight per day, which may be administered in single or multiple doses. Preferably, the dosage level will be about 0.001 mg per kg to about 100 mg per kg per day; more preferably about 0.01 mg per kg to about 50 mg per kg per day; even more preferably about 0.1 mg per kg to about 10 mg per kg subject body weight.

A combination therapy comprising a muscarinic receptor antagonist that is intended for the oral administration of humans may contain from about 10 micrograms to about 10 grams of active agent optionally compounded with an appropriate and convenient amount of carrier material, which may vary from about 5 to about 95 percent of the total composition. More preferably, the muscarinic receptor antagonist is dosed at between about 0.1 mg and about 1 gram. Even more preferably, the muscarinic receptor antagonist is dosed at between about 1 mg and about 750 mg. Even more preferably still, the muscarinic receptor antagonist is dosed at between about 100 mg and about 500 mg.

The exact dosage and regimen for administering a Cox-2 inhibitor in combination with a muscarinic receptor antagonist will necessarily depend upon the potency and duration of action of the compounds used, the nature and severity of the illness to be treated, as well as the sex, age, weight, general health, and individual responsiveness of the patient to be treated, and other relevant circumstances. Those skilled in the art will appreciate that dosages may also be determined with guidance from Goodman & Gilman's The Pharmacological Basis of Therapeutics, Ninth Edition (1996), Appendix II, pp. 1707-1711.

The effectiveness of a particular dosage of a Cox-2 inhibitor in combination with a muscarinic receptor antagonist is determined by monitoring the effect of a given dosage on the progress or prevention of a particular respiratory disorder must be monitored.

Because early COPD may produce no visible symptoms or signs, laboratory tests can be used to diagnose and/or follow the presence or degree of airflow obstruction. For example, the degree and severity of asthma and COPD can be determined by measuring lung expiratory flow volume and expiratory flow rates. Such a measurement can be accomplished with, for example, a spirometer, flow volume loop, or pneumotach, before and after each of the treatments. The use of spirometry can be a standard test for determining the efficacy of a combination of Cox-2 inhibitors and muscarinic receptor antagonists after administration to a subject suffering from a pulmonary inflammatory disorder.

Spirometry is a medical test that measures the physical volume of air an individual forcibly inhales or exhales into a device. The objective of spirometry is to assess ventilatory function. A device called a spirometer is used to measure how much air the lungs can hold and how well the respiratory system is able to move air into and out of the lungs. An estimate of flow rate, or the rate at which the volume is changing as a function of time can also be calculated with spirometery. See College of Physicians and Surgeons of Alberta, “Guidelines For Spirometry & Flow Volume Measurements”(1998).

<www.cpsa.ab.ca/qoc/Guidelines%20for%20Spirometry%20&%20Flow%20Volume%20Measurements.doc>.

Common parameters that spirometry measures are Forced Vital Capacity (FVC)—the maximum volume of air, measured in liters that can be forcibly and rapidly exhaled. Another parameter is Forced Expiratory Volume (FEV1)—the volume of air expelled in the first second of a forced expiration. Normal parameters for a subject not suffering from an inflammatory disorder such as asthma or COPD is: Tidal volume —5 to 7 milliliters per kilogram of body weight; Expiratory reserve volume —25% of vital capacity; Inspiratory capacity—75% of vital capacity forced expiratory volume—75% of vital capacity after 1 second, 94% after 2 seconds, and 97% after 3 seconds. Healthatoz.com, wellness, test & procedures, spirometry <http://www.healthatoz.com/atoz/TestProcedures/TPspirometry.html>.

Spirometry results are expressed as a percentage, and are considered abnormal if less than 80% of the normal predicted value. An abnormal result usually indicates the presence of some degree of obstructive lung disease such as COPD and chronic bronchitis, or restrictive lung disease such as pulmonary fibrosis or asthma. For example, an abnormally low FEV1.0/FVC means that a subject's airflow is obstructed. If someone has COPD, a low FEV1.0 not only reveals that the person has obstructive lung disease, but measures how severe the obstruction is.

Thus, with the methods and compositions of the present invention, spirometric comparisons of pulmonary airflow in a subject suffering from a respiratory disorder before and after treatment will elucidate similarities and differences that enable one of skill to determine the effectiveness of the treatment methods.

As used herein, the term “subject” for purposes of treatment includes any subject, and preferably is a subject who is in need of the treatment of respiratory disorders, or who needs treatment of a respiratory disorder-related complication. For purposes of prevention, the subject is any subject, and preferably is a subject that is at risk for, or is predisposed to, developing a respiratory disorder or a respiratory disorder-related complication. The subject is typically an animal, and yet more typically is a mammal. “Mammal”, as that term is used herein, refers to any animal classified as a mammal, including humans, domestic and farm animals, zoo, sports, or pet animals, such as dogs, horses, cats, cattle, etc. Preferably, the mammal is a human. For purposes of the present invention, an adult human weighs approximately seventy kilograms.

As used herein, the terms “predisposed to” and “at risk for,” both of which are used interchangeably herein, mean any subject at risk for developing respiratory disorders or any respiratory disorder-related complication. The subject may be a human subject who is at risk for developing respiratory disorders or a respiratory disorder-related complication. The subject may be at risk due to genetic predisposition, diet, age, exposure to a lung truama, exposure to a potentially traumatic environment, exposure to respiratory disorder-causing agents, such as cigarette smoke, and the like. The subject may also be at risk due to physiological factors such as anatomical and biochemical abnormalities in the lung.

As used herein, the terms “subject is one that is in need of the prevention or treatment of a respiratory disorder or a respiratory disorder-related complication” refer to any subject who is suffering from or is predisposed to respiratory disorders or respiratory disorder-related complication described herein. The terms “subject is one that is in need of the prevention or treatment of a respiratory disorder or a respiratory disorder-related complication” also refer to any subject that requires a lower dose of conventional respiratory disorder treatment agents. In addition, the terms “subject is one that is in need of the prevention or treatment of a respiratory disorder or a respiratory disorder-related complication” means any subject who requires a reduction in the side effects of a conventional respiratory disorder treatment agent. Furthermore, the terms “subject is one that is in need of the prevention or treatment of a respiratory disorder or a respiratory disorder-related complication” means any subject who requires improved tolerability to any conventional respiratory disorder treatment agent for respiratory disorders therapy.

In other embodiments, the present invention encompasses a kit comprising one dosage form comprising a Cox-2 inhibitor and a second dosage form comprising a muscarinic receptor antagonist.

A therapy comprising a Cox-2 inhibitor in combination with a muscarinic receptor antagonist encompasses the treatment and prevention of such respiratory disorder symptoms as, for example, coughing, inflammation, congestion, dyspnea, wheezing, hyperventilation, difficulty breathing, bronchospasm, and bronchoconstriction dyspnea, fluid accumulation within the lung, and difficulty breathing in a subject suffering from such symptoms.

As used herein, the terms “respiratory disorder” is defined as having any disorder or disease of the lung, throat, mouth, nose, or sinus cavity or even a post-surgical condition of the lung, throat, mouth, nose, or sinus cavity. Respiratory disorders include any condition of the lung or airways that does not normally occur in or on the airways. As used herein, the term “airway” includes any component or structure found within or on the lung, throat, mouth, nose, or sinus cavity.

The terms “respiratory disorder” also include any complications that arise from having such a disorder. For example, lung abscesses may develop from a prolonged untreated respiratory disorder, such as COPD, lung cancer, or tuberculosis. Thus, the terms “respiratory disorder,” “respiratory disorder complication” and “respiratory disorder-related complication,” used interchangeably herein, includes any subsequent disease, disorder, injury or condition that may arise from having a respiratory disorder. The term “respiratory disorder-related complication” refers to any condition where developing a respiratory disorder is a risk factor for developing additional health complications.

The respiratory disorders that may be treated with the compositions and methods described herein, include one or more of, but are not limited to asthma, COPD, bronchitis, chronic bronchitis, acute bronchitis, rhinitis, cystic fibrosis, tuberculosis, pneumonia, lung cancer, tracheal cancer, chronic obstructive bronchitis, emphysema, adult respiratory distress syndrome, respiratory failure, bronchiectasis, atelectasis, pulmonary embolism, occupational lung diseases, Goodpasture's Syndrome, idiopathic interstitial lung diseases, pulmonary alveolar proteinosis, giant bullae, Legionnaires' disease, psittacosis, pulmonary fibrosis, interstitial pneumonia, pleurisy, pleural effusion, pleural fibrosis, pneumothorax, postoperative and posttraumatic injury, postoperative and posttraumatic pneumonia, and pleural disorders.

The methods and compositions of the present invention also encompass the prevention and treatment of the respiratory disorder, COPD.

As used herein, the term “chronic obstructive pulmonary disease” or “COPD” refers to a set of physiological symptoms including chronic cough, expectoration, exertional dyspnea and a significant, progressive reduction in airflow that may or may not be partly reversible. COPD is a disease characterized by a progressive airflow limitation caused by an abnormal inflammatory reaction to the chronic inhalation of particles.

In subjects with the disorder, poor gas exchange in the lungs leads to decreased oxygen levels in the blood, increased levels of carbon dioxide and shortness of breath. Chronic airflow obstruction in COPD is complicated by the loss of lung elasticity resulting from enzymatic destruction of the lung parenchyma. Rather than a single pathologic condition, COPD is an umbrella term encompassing chronic obstructive bronchitis and emphysema.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims, which follow the examples. In the examples, all percentages are given on a weight basis unless otherwise indicated.

### EXAMPLE 1

This example shows the preparation of the Cox-2 inhibitor, celecoxib.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇H₁₄N₃O₂SF₃; C, 53.54; H, 3.70; N, 11.02. The composition that was found by analysis was: C, 53.17; H, 3.81; N, 10.90.

### EXAMPLE 2

This example shows a general outline for the synthesis of the muscarinic receptor antagonist, tiotropium bromide.

The Grignard condensation of oxalic acid dimethyl ester (I—from the outline depicted below) with 2-thienylmagnesium bromide (II) gives 2-hydroxy-2,2-bis(2-thienyl)acetic acid methyl ester (III), which is submitted to transesterification with scopine (IV) catalyzed by Na or NaOEt, providing the corresponding ester (V). Finally, (V) is quaternized with methyl bromide in dichloromethane/acetonitrile (1).

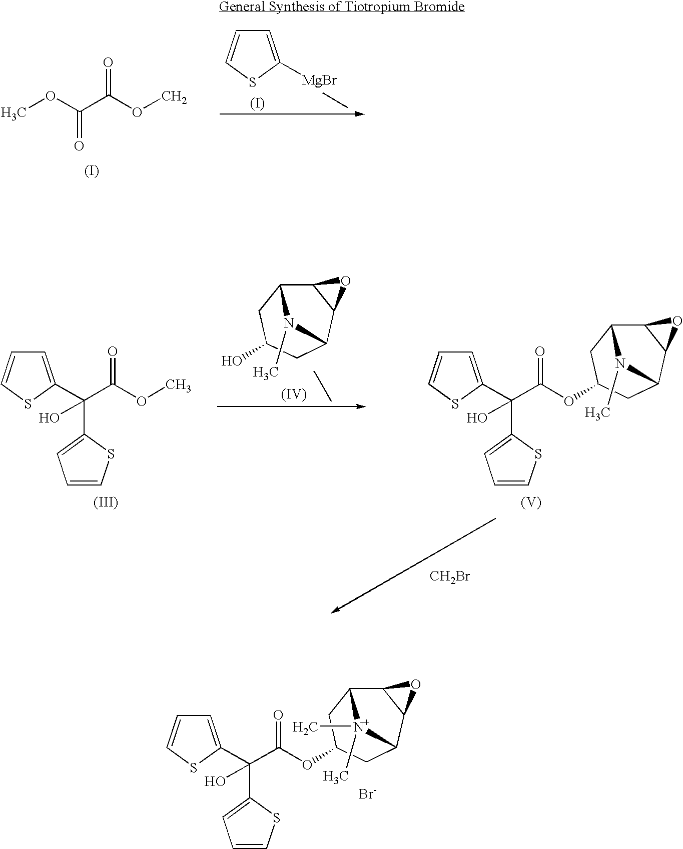

Therapeutic and pharmaceutical compositions comprising a combination of any of the Cox-2 inhibitors and any of the muscarinic receptor antagonist active ingredients that are described above can be formed by similar methods.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense. In addition, it should be understood that aspects of the various embodiments may be interchanged both in whole or in part.

1. A method of preventing or treating respiratory disorders and respiratory disorder-related complications in a subject comprising administering to the subject a Cox-2 inhibitor in combination with a muscarinic receptor antagonist.

2. The method according to claim 1, wherein the subject is one that is in need of the prevention or treatment of a respiratory disorder or a respiratory disorder-related complication.

3. The method according to claim 1, wherein the Cox-2 inhibitor comprises a non-steroidal anti-inflammatory drug.

4. The method according to claim 1, wherein the Cox-2 inhibitor is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester, and mixtures thereof.

5. The method according to claim 1, wherein the Cox-2 inhibitor comprises a Cox-2 selective inhibitor.

6. The method according to claim 5, wherein the Cox-2 selective inhibitor is selected from the group consisting of celecoxib, parecoxib, deracoxib, valdecoxib, meloxicam, rofecoxib, lumiracoxib, etoricoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

7. The method according to claim 5, wherein the Cox-2 selective inhibitor comprises a tricyclic Cox-2 selective inhibitor.

8. The method according to claim 7, wherein the tricyclic Cox-2 selective inhibitor is selected from the group consisting of celecoxib, parecoxib, deracoxib, valdecoxib, etoricoxib, rofecoxib, prodrugs of any of them, and mixtures thereof.

9. The method according to claim 8, wherein the Cox-2 selective inhibitor comprises celecoxib.

10. The method according to claim 4, wherein the Cox-2 selective inhibitor is a chromene Cox-2 selective inhibitor.

11. The method according to claim 10, wherein the chromene Cox-2 selective inhibitor comprises at least one compound that is selected from the group consisting of:
6-chloro-2-trifluoromethyl-2H-1-benzopyran-3-carboxylic acid,
6-chloro-7-methyl-2-trifluoromethyl-2H-1-benzopyran-3-carboxylic acid,
8-(1-methylethyl)-2-trifluoromethyl-2H-1-benzopyran-3-carboxylic acid,
6-chloro-7-(1,1-dimethylethyl)-2-trifluoromethyl-2H-1-benzopyran-3-carboxylic acid,
6-chloro-8-(1-methylethyl)-2-trifluoromethyl-2H-1-benzopyran-3-carboxylic acid,
2-trifluoromethyl-3H-naphthopyran-3-carboxylic acid,
7-(1,1-dimethylethyl)-2-trifluoromethyl-2H-1-benzopyran-3-carboxylic acid,
6-bromo-2-trifluoromethyl-2H-1-benzopyran-3-carboxylic acid,
8-chloro-2-trifluoromethyl-2H-1-benzopyran-3-carboxylic acid,
6-trifluoromethoxy-2-trifluoromethyl-2H-1-benzopyran-3-carboxylic acid,
5,7-dichloro-2-trifluoromethyl-2H-1-benzopyran-3-carboxylic acid,
8-phenyl-2-trifluoromethyl-2H-1-benzopyran-3-carboxylic acid,
7,8-dimethyl-2-trifluoromethyl-2H-1-benzopyran-3-carboxylic acid,
6,8-bis(dimethylethyl)-2-trifluoromethyl-2H-1-benzopyran-3-carboxylic acid,
7-(1-methylethyl)-2-trifluoromethyl-2H-1-benzopyran-3-carboxylic acid,
7-phenyl-2-trifluoromethyl-2H-1-benzopyran-3-carboxylic acid,
6-chloro-7-ethyl-2-trifluoromethyl-2H-1-benzopyran-3-carboxylic acid,
6-chloro-8-ethyl-2-trifluoromethyl-2H-1-benzopyran-3-carboxylic acid,
6-chloro-7-phenyl-2-trifluoromethyl-2H-1-benzopyran-3-carboxylic acid,
6,7-dichloro-2-trifluoromethyl-2H-1-benzopyran-3-carboxylic acid,
6,8-dichloro-2-trifluoromethyl-2H-1-benzopyran-3-carboxylic acid,
2-trifluoromethyl-3H-naptho[2,1-b]pyran-3-carboxylic acid,
6-chloro-8-methyl-2-trifluoromethyl-2H-1-benzopyran-3-carboxylic acid,
8-chloro-6-methyl-2-trifluoromethyl-2H-1-benzopyran-3-carboxylic acid,
8-chloro-6-methoxy-2-trifluoromethyl-2H-1-benzopyran-3-carboxylic acid,
6-bromo-8-chloro-2-trifluoromethyl-2H-1-benzopyran-3-carboxylic acid,
8-bromo-6-fluoro-2-trifluoromethyl-2H-1-benzopyran-3-carboxylic acid,
8-bromo-6-methyl-2-trifluoromethyl-2H-1-benzopyran-3-carboxylic acid,
8-bromo-5-fluoro-2-trifluoromethyl-2H-1-benzopyran-3-carboxylic acid,
6-chloro-8-fluoro-2-trifluoromethyl-2H-1-benzopyran-3-carboxylic acid,
6-bromo-8-methoxy-2-trifluoromethyl-2H-1-benzopyran-3-carboxylic acid,
6-[[(phenylmethyl)amino]sulfonyl]-2-trifluoromethyl-2H-1-benzopyran-3-carboxylic acid,
6-[(dimethylamino)sulfonyl]-2-trifluoromethyl-2H-1-benzopyran-3-carboxylic acid,
6-[(methylamino)sulfonyl]-2-trifluoromethyl-2H-1-benzopyran-3-carboxylic acid,
6-[(4-morpholino)sulfonyl]-2-trifluoromethyl-2H-1-benzopyran-3-carboxylic acid,
6-[(1,1-dimethylethyl)aminosulfonyl]-2-trifluoromethyl-2H-1-benzopyran-3-carboxylic acid,
6-[(2-methylpropyl)aminosulfonyl]-2-trifluoromethyl-2H-1-benzopyran-3-carboxylic acid,
6-methylsulfonyl-2-trifluoromethyl-2H-1-benzopyran-3-carboxylic acid,
8-chloro-6-[[(phenylmethyl)amino]sulfonyl]-2-trifluoromethyl-2H-1-benzopyran-3-carboxylic acid,
6-phenylacetyl-2-trifluoromethyl-2H-1-benzopyran-3-carboxylic acid,
6,8-dibromo-2-trifluoromethyl-2H-1-benzopyran-3-carboxylic acid,
8-chloro-5,6-dimethyl-2-trifluoromethyl-2H-1-benzopyran-3-carboxylic acid,
6,8-dichloro-(S)-2-trifluoromethyl-2H-1-benzopyran-3-carboxylic acid,
6-benzylsulfonyl-2-trifluoromethyl-2H-1-benzopyran-3-carboxylic acid,
6-[[N-(2-furylmethyl)amino]sulfonyl]-2-trifluoromethyl-2H-1-benzopyran-3-carboxylic acid,
6-[[N-(2-phenylethyl)amino]sulfonyl]-2-trifluoromethyl-2H-1-benzopyran-3-carboxylic acid,
6-iodo-2-trifluoromethyl-2H-1-benzopyran-3-carboxylic acid,
7-(1,1-dimethylethyl)-2-pentafluoroethyl-2H-1-benzopyran-3-carboxylic acid,
6-chloro-2-trifluoromethyl-2H-1-benzothiopyran-3-carboxylic acid.
6-chloro-2-trifluoromethyl-2H-1-benzopyran-3-carboxylic acid,
(S)-6-chloro-2-trifluoromethyl-2H-1-benzopyran-3-carboxylic acid,
6-chloro-7-(1,1-dimethylethyl)-2-trifluoromethyl-2H-1-benzopyran-3-carboxylic acid,
(S)-6-chloro-7-(1,1-dimethylethyl)-2-(trifluoromethyl)-2H-1-benzopyran-3-carboxylic acid,
6-trifluoromethoxy-2-trifluoromethyl-2H-1-benzopyran-3-carboxylic acid,
(S)-6-trifluoromethoxy-2-trifluoromethyl-2H-1-benzopyran-3-carboxylic acid,
6-formyl-2-(trifluoromethyl)-2H-1-benzopyran-3-carboxylic acid,
6-(difluoromethyl)-2-(trifluoromethyl)-2H-1-benzopyran-3-carboxylic acid,
6,8-dichloro-7-methyl-2-(trifluoromethyl)-2H-1-benzopyran-3-carboxylic acid,
6,8-dichloro-2-trifluoromethyl-2H-1-benzopyran-3-carboxylic acid,
(S)-6,8-dichloro-2-(trifluoromethyl)-2H-1-benzopyran-3-carboxylic acid,
6-chloro-1,2-dihydro-2-(trifluoromethyl)-3-quinolinecarboxylic acid,
(S)-6-chloro-1,2-dihydro-2-(trifluoromethyl)-3-quinolinecarboxylic acid,
6,8-dichloro-1,2-dihydro-2-(trifluoromethyl)-3-quinolinecarboxylic acid,
7-(1,1-dimethylethyl)-2-trifluoromethyl-2H-1-benzopyran-3-carboxylic acid,
6,7-dichloro-2-trifluoromethyl-2H-1-benzopyran-3-carboxylic acid,
5,6-dichloro-2-(trifluoromethyl)-2H-1-benzopyran-3-carboxylic acid,
2,6-bis(trifluoromethyl)-2H-1-benzopyran-3-carboxylic acid,
5,6,7-trichloro-2-(trifluoromethyl)-2H-1-benzopyran-3-carboxylic acid,
6,7,8-trichloro-2-(trifluoromethyl)-2H-1-benzopyran-3-carboxylic acid,
6-iodo-1,2-dihydro-2-(trifluoromethyl)-3-quinolinecarboxylic acid,
6-bromo-1,2-dihydro-2-(trifluoromethyl)-3-quinolinecarboxylic acid,
6-chloro-7-methyl-2-(trifluoromethyl)-2H-1-benzothiopyran-3-carboxylic acid,
6,8-dichloro-2-trifluoromethyl-2H-1-benzothiopyran-3-carboxylic acid, and mixtures thereof.

12. The method according to claim 10, wherein the chromene Cox-2 selective inhibitor is selected from the group consisting of
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid, and
(2S)-6-chloro-5,7-dimethyl-2-(trifluoromethyl)-2H-chromene-3-carboxylic acid, and mixtures thereof.

13. The method according to claim 1, wherein the muscarinic receptor antagonist is a non-selective muscarinic receptor antagonist.

14. The method according to claim 1, wherein the muscarinic receptor antagonist is a selective muscarinic receptor antagonist.

15. The method according to claim 1, wherein the muscarinic receptor antagonist is a selective muscarinic receptor antagonist that is selective for the muscarinic receptor subtypes M₁ and M₃.

16. The method according to claim 1, wherein the muscarinic receptor antagonist is selected from the group consisting of tiotropium bromide, butyiscopolamine bromide, quinuclidinyl benzilate quinuclidinyl-a-hydroxydiphenylacetate, 1,1-dimethyl-4-diphenylacetoxypiperidinium iodide, ipratropium bromide, nitrocaramiphen hydrochloride, pirenzepine dihydrochloride, scopolamine hydrobromide, telenzepine dihydrochloride, tropicamide, hexamethylene-bis-[dimethyl-(3-phthalimidopropyl) ammonium]bromide, atropine sulfate, glycopyrrolate, scopolamine, benztropine mesylate (1S, 3′R)-quinuclidin-3′-yl 1-phenyl-1, 2, 3, 4-tetrahydroisoquinoline-2-carboxylate, tripitramine, cyclopentolate hydrochloride, clozapine, (2R)-N-[1-(4-methyl-3-pentenyl)piperidin-4-yl]-2-cyclopentyl-2-hydroxy-2-phenylacetamide, (+/−)-terodiline, methoctramine tetrahydrochloride, (2R)-2-[(1R)-3,3-difluorocyclopentyl]-2-hydroxy-2-phenylacetamide, 2-diisopropyl aminoethyl 1-phenylcyclopentane carboxylate hydrochloride, tolterodine tartrate, cyclohexylmethyl-piperidinyltriphenyl-propioamide, 4-cyclohexyl-alpha-[4-[[4-methoxyphenyl]sulfinyl]-phenyl]-1-piperazine acetonitrile, tiquizium bromide, oxitropium bromide, pirenzepine dihydrochloride, revatropate, AQ-RA 721, DAC 5889, hyoscyamine sulfate, oxybutynin, propantheline bromide, flavoxate, dicyclomine, PD 102807, gallamine triethiodide, himbacine, otenzepad, 5,11-dihydro-11-[2-[2-[(N,N-dipropylaminomethyl)piperidin-1-yl]ethylamino]-carbonyl]6H-pyrido[2,3-B][1,4]benzodiazepin-6-one, pfluorohexahydro-sila-difenidol hydrochloride, cyclohexyl-(4-fluorophenyl)-(3-N-piperidinopropyl)silanol hydrochloride, olanzapine, methscolpamine bromide, trihexyphenidyl hydrochloride, darifenacin, homatropine hydrobromide, 2-ethyl-5-(1-methyl-1,2,5,6-tetrahydropyridyl)-2H-tetrazole maleate, 1,3-dihydro-1-{1-[piperidin-4-yl]piperidin-4-yl}-2H-benzimidizol-2-ones, 1,3-dihydro-1-{4-amino-1-cyclohexyl}-2H-benzimidazol-2-ones, benzocycloalkylenylamine derivatives, fesoterodine, (2R)-N-[1-(6-aminopyridin-2-ylmethyl)piperidin-4-yl]-2-[(1R)-3,3-difluorocyclopentyl]-2-hydroxy-2-phenylacetamide, 2-[(1S,3S)-3-sulfonylaminocyclopentyl]phenylacetamide derivatives, zamifenacin, (2S, 3′R) 3-quinuclidinyl tropate, 3-quinuclidinyl 3-hydroxymethyl 2-phenyl alkanoates, 3-quinuclidinyl 3-hydroxymethyl 2-thienyl alkanoates, 1-azabicyclo[2.2.2]octan-3-yl 2-aryl-3-azacyclo-2-hydroxypropionates, 3-quinuclidinol esters, unsymmetrical alpha-disubstituted glycolic acids, benztropine, methscopolamine, KRP-197, and tropicamide, prodrugs of any of them, and mixtures thereof.

17. The method according to claim 1, wherein the muscarinic receptor antagonist is tiotropium bromide.

18. The method according to claim 1, wherein the subject suffers from or is predisposed to one or more respiratory disorders selected from the group consisting of asthma, COPD, bronchitis, chronic bronchitis, acute bronchitis, rhinitis, cystic fibrosis, tuberculosis, pneumonia, lung cancer, tracheal cancer, chronic obstructive bronchitis, emphysema, adult respiratory distress syndrome, respiratory failure, bronchiectasis, atelectasis, pulmonary embolism, occupational lung diseases, Goodpasture's Syndrome, idiopathic interstitial lung diseases, pulmonary alveolar proteinosis, giant bullae, Legionnaires' disease, psittacosis, pulmonary fibrosis, interstitial pneumonia, pleurisy, pleural effusion, pleural fibrosis, pneumothorax, postoperative and posttraumatic injury, postoperative and posttraumatic pneumonia, and pleural disorders.

19. The method according to claim 1, wherein the subject suffers from, or is predisposed to, COPD.

20. The method according to claim 1, further comprising administering an amount of a Cox-2 inhibitor and an amount of a muscarinic receptor antagonist wherein the amount of the Cox-2 inhibitor and the amount of the muscarinic receptor antagonist together comprise a therapeutically effective amount.

21. A therapeutic composition comprising at least one Cox-2 inhibitor and one or more muscarinic receptor antagonists.

22. The therapeutic composition according to claim 21, wherein the Cox-2 inhibitor comprises a Cox-2 selective inhibitor.

23. The therapeutic composition according to claim 22, wherein the Cox-2 selective inhibitor is selected from the group consisting of celecoxib, parecoxib, deracoxib, valdecoxib, meloxicam, rofecoxib, lumiracoxib, etoricoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

24. The therapeutic composition according to claim 22, wherein the Cox-2 selective inhibitor comprises a tricyclic Cox-2 selective inhibitor.

25. The therapeutic composition according to claim 24, wherein the tricyclic Cox-2 selective inhibitor is selected from the group consisting of celecoxib, parecoxib, deracoxib, valdecoxib, etoricoxib, rofecoxib, prodrugs of any of them, and mixtures thereof.

26. The therapeutic composition according to claim 22, wherein the Cox-2 selective inhibitor comprises celecoxib.

27. The therapeutic composition according to claim 4, wherein the Cox-2 selective inhibitor is a chromene Cox-2 selective inhibitor.

28. The therapeutic composition according to claim 27, wherein the chromene Cox-2 selective inhibitor comprises at least one compound that is selected from the group consisting of:
6-chloro-2-trifluoromethyl-2H-1-benzopyran-3-carboxylic acid,
6-chloro-7-methyl-2-trifluoromethyl-2H-1-benzopyran-3-carboxylic acid,
8-(1-methylethyl)-2-trifluoromethyl-2H-1-benzopyran-3-carboxylic acid,
6-chloro-7-(1,1-dimethylethyl)-2-trifluoromethyl-2H-1-benzopyran-3-carboxylic acid,
6-chloro-8-(1-methylethyl)-2-trifluoromethyl-2H-1-benzopyran-3-carboxylic acid,
2-trifluoromethyl-3H-naphthopyran-3-carboxylic acid,
7-(1,1-dimethylethyl)-2-trifluoromethyl-2H-1-benzopyran-3-carboxylic acid,
6-bromo-2-trifluoromethyl-2H-1-benzopyran-3-carboxylic acid,
8-chloro-2-trifluoromethyl-2H-1-benzopyran-3-carboxylic acid,
6-trifluoromethoxy-2-trifluoromethyl-2H-1-benzopyran-3-carboxylic acid,
5,7-dichloro-2-trifluoromethyl-2H-1-benzopyran-3-carboxylic acid,
8-phenyl-2-trifluoromethyl-2H-1-benzopyran-3-carboxylic acid,
7,8-dimethyl-2-trifluoromethyl-2H-1-benzopyran-3-carboxylic acid,
6,8-bis(dimethylethyl)-2-trifluoromethyl-2H-1-benzopyran-3-carboxylic acid,
7-(1-methylethyl)-2-trifluoromethyl-2H-1-benzopyran-3-carboxylic acid,
7-phenyl-2-trifluoromethyl-2H-1-benzopyran-3-carboxylic acid,
6-chloro-7-ethyl-2-trifluoromethyl-2H-1-benzopyran-3-carboxylic acid,
6-chloro-8-ethyl-2-trifluoromethyl-2H-1-benzopyran-3-carboxylic acid,
6-chloro-7-phenyl-2-trifluoromethyl-2H-1-benzopyran-3-carboxylic acid,
6,7-dichloro-2-trifluoromethyl-2H-1-benzopyran-3-carboxylic acid,
6,8-dichloro-2-trifluoromethyl-2H-1-benzopyran-3-carboxylic acid,
2-trifluoromethyl-3H-naptho[2,1-b]pyran-3-carboxylic acid,
6-chloro-8-methyl-2-trifluoromethyl-2H-1-benzopyran-3-carboxylic acid,
8-chloro-6-methyl-2-trifluoromethyl-2H-1-benzopyran-3-carboxylic acid,
8-chloro-6-methoxy-2-trifluoromethyl-2H-1-benzopyran-3-carboxylic acid,
6-bromo-8-chloro-2-trifluoromethyl-2H-1-benzopyran-3-carboxylic acid,
8-bromo-6-fluoro-2-trifluoromethyl-2H-1-benzopyran-3-carboxylic acid,
8-bromo-6-methyl-2-trifluoromethyl-2H-1-benzopyran-3-carboxylic acid,
8-bromo-5-fluoro-2-trifluoromethyl-2H-1-benzopyran-3-carboxylic acid,
6-chloro-8-fluoro-2-trifluoromethyl-2H-1-benzopyran-3-carboxylic acid,
6-bromo-8-methoxy-2-trifluoromethyl-2H-1-benzopyran-3-carboxylic acid,
6-[[(phenylmethyl)amino]sulfonyl]-2-trifluoromethyl-2H-1-benzopyran-3-carboxylic acid,
6-[(dimethylamino)sulfonyl]-2-trifluoromethyl-2H-1-benzopyran-3-carboxylic acid,
6-[(methylamino)sulfonyl]-2-trifluoromethyl-2H-1-benzopyran-3-carboxylic acid,
6-[(4-morpholino)sulfonyl]-2-trifluoromethyl-2H-1-benzopyran-3-carboxylic acid,
6-[(1,1-dimethylethyl)aminosulfonyl]-2-trifluoromethyl-2H-1-benzopyran-3-carboxylic acid,
6-[(2-methylpropyl)aminosulfonyl]-2-trifluoromethyl-2H-1-benzopyran-3-carboxylic acid,
6-methylsulfonyl-2-trifluoromethyl-2H-1-benzopyran-3-carboxylic acid,
8-chloro-6-[[(phenylmethyl)amino]sulfonyl]-2-trifluoromethyl-2H-1-benzopyran-3-carboxylic acid,
6-phenylacetyl-2-trifluoromethyl-2H-1-benzopyran-3-carboxylic acid,
6,8-dibromo-2-trifluoromethyl-2H-1-benzopyran-3-carboxylic acid,
8-chloro-5,6-dimethyl-2-trifluoromethyl-2H-1-benzopyran-3-carboxylic acid,
6,8-dichloro-(S)-2-trifluoromethyl-2H-1-benzopyran-3-carboxylic acid,
6-benzylsulfonyl-2-trifluoromethyl-2H-1-benzopyran-3-carboxylic acid,
6-[[N-(2-furylmethyl)amino]sulfonyl]-2-trifluoromethyl-2H-1-benzopyran-3-carboxylic acid,
6-[[N-(2-phenylethyl)amino]sulfonyl]-2-trifluoromethyl-2H-1-benzopyran-3-carboxylic acid,
6-iodo-2-trifluoromethyl-2H-1-benzopyran-3-carboxylic acid,
7-(1,1-dimethylethyl)-2-pentafluoroethyl-2H-1-benzopyran-3-carboxylic acid,
6-chloro-2-trifluoromethyl-2H-1-benzothiopyran-3-carboxylic acid.
6-chloro-2-trifluoromethyl-2H-1-benzopyran-3-carboxylic acid,
(S)-6-chloro-2-trifluoromethyl-2H-1-benzopyran-3-carboxylic acid,
6-chloro-7-(1,1-dimethylethyl)-2-trifluoromethyl-2H-1-benzopyran-3-carboxylic acid,
(S)-6-chloro-7-(1,1-dimethylethyl)-2-(trifluoromethyl)-2H-1-benzopyran-3-carboxylic acid,
6-trifluoromethoxy-2-trifluoromethyl-2H-1-benzopyran-3-carboxylic acid,
(S)-6-trifluoromethoxy-2-trifluoromethyl-2H-1-benzopyran-3-carboxylic acid,
6-formyl-2-(trifluoromethyl)-2H-1-benzopyran-3-carboxylic acid,
6-(difluoromethyl)-2-(trifluoromethyl)-2H-1-benzopyran-3-carboxylic acid,
6,8-dichloro-7-methyl-2-(trifluoromethyl)-2H-1-benzopyran-3-carboxylic acid,
6,8-dichloro-2-trifluoromethyl-2H-1-benzopyran-3-carboxylic acid,
(S)-6,8-dichloro-2-(trifluoromethyl)-2H-1-benzopyran-3-carboxylic acid,
6-chloro-1,2-dihydro-2-(trifluoromethyl)-3-quinolinecarboxylic acid,
(S)-6-chloro-1,2-dihydro-2-(trifluoromethyl)-3-quinolinecarboxylic acid,
6,8-dichloro-1,2-dihydro-2-(trifluoromethyl)-3-quinolinecarboxylic acid,
7-(1,1-dimethylethyl)-2-trifluoromethyl-2H-1-benzopyran-3-carboxylic acid,
6,7-dichloro-2-trifluoromethyl-2H-1-benzopyran-3-carboxylic acid,
5,6-dichloro-2-(trifluoromethyl)-2H-1-benzopyran-3-carboxylic acid,
2,6-bis(trifluoromethyl)-2H-1-benzopyran-3-carboxylic acid,
5,6,7-trichloro-2-(trifluoromethyl)-2H-1-benzopyran-3-carboxylic acid,
6,7,8-trichloro-2-(trifluoromethyl)-2H-1-benzopyran-3-carboxylic acid,
6-iodo-1,2-dihydro-2-(trifluoromethyl)-3-quinolinecarboxylic acid,
6-bromo-1,2-dihydro-2-(trifluoromethyl)-3-quinolinecarboxylic acid,
6-chloro-7-methyl-2-(trifluoromethyl)-2H-1-benzothiopyran-3-carboxylic acid,
6,8-dichloro-2-trifluoromethyl-2H-1-benzothiopyran-3-carboxylic acid, and mixtures thereof.

29. The therapeutic composition according to claim 27, wherein the chromene Cox-2 selective inhibitor is selected from the group consisting of
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid, and
(2S)-6-chloro-5,7-dimethyl-2-(trifluoromethyl)-2H-chromene-3-carboxylic acid, and mixtures thereof.

30. The therapeutic composition according to claim 21, wherein the muscarinic receptor antagonist is a non-selective muscarinic receptor antagonist.

31. The therapeutic composition according to claim 21, wherein the muscarinic receptor antagonist is a selective muscarinic receptor antagonist.

32. The therapeutic composition according to claim 21, wherein the muscarinic receptor antagonist is a selective muscarinic receptor antagonist that is selective for the muscarinic receptor subtypes M₁ and M₃.

33. The therapeutic composition according to claim 21, wherein the muscarinic receptor antagonist is selected from the group consisting of tiotropium bromide, butylscopolamine bromide, quinuclidinyl benzilate quinuclidinyl-a-hydroxydiphenylacetate, 1,1-dimethyl-4-diphenylacetoxypiperidinium iodide, ipratropium bromide, nitrocaramiphen hydrochloride, pirenzepine dihydrochloride, scopolamine hydrobromide, telenzepine dihydrochloride, tropicamide, hexamethylene-bis-[dimethyl-(3-phthalimidopropyl) ammonium]bromide, atropine sulfate, glycopyrrolate, scopolamine, benztropine mesylate (1S, 3′R)-quinuclidin-3′-yl 1-phenyl-1, 2, 3, 4-tetrahydroisoquinoline-2-carboxylate, tripitramine, cyclopentolate hydrochloride, clozapine, (2R)-N-[1-(4-methyl-3-pentenyl)piperidin-4-yl]-2-cyclopentyl-2-hydroxy-2-phenylacetamide, (+/−)-terodiline, methoctramine tetrahydrochloride, (2R)-2-[(1R)-3,3-difluorocyclopentyl]-2-hydroxy-2-phenylacetamide, 2-diisopropyl aminoethyl 1-phenylcyclopentane carboxylate hydrochloride, tolterodine tartrate, cyclohexylmethyl-piperidinyltriphenyl-propioamide, 4-cyclohexyl-alpha-[4-[[4-methoxyphenyl]sulfinyl]-phenyl]-1-piperazine acetonitrile, tiquizium bromide, oxitropium bromide, pirenzepine dihydrochloride, revatropate, AQ-RA 721, DAC 5889, hyoscyamine sulfate, oxybutynin, propantheline bromide, flavoxate, dicyclomine, PD 102807, gallamine triethiodide, himbacine, otenzepad, 5,11-dihydro-11-[2-[2-[(N,N-dipropylaminomethyl)piperidin-1-yl]ethylamino]-carbonyl]6h-pyrido[2,3-B][1,4]benzodiazepin-6-one, p-fluorohexahydro-sila-difenidol hydrochloride, cyclohexyl-(4-fluorophenyl)-(3-N-piperidinopropyl)silanol hydrochloride, olanzapine, methscolpamine bromide, trihexyphenidyl hydrochloride, darifenacin, homatropine hydrobromide, 2-ethyl-5-(1-methyl-1,2,5,6-tetrahydropyridyl)-2H-tetrazole maleate, 1,3-dihydro-1-{1-[piperidin-4-yl]piperidin-4-yl}-2H-benzimidizol-2-ones, 1,3-dihydro-1-{4-amino-1-cyclohexyl}-2H-benzimidazol-2-ones, benzocycloalkylenylamine derivatives, fesoterodine, (2R)-N-[1-(6-aminopyridin-2-ylmethyl)piperidin-4-yl]-2-[(1R)-3,3-difluorocyclopentyl]-2-hydroxy-2-phenylacetamide, 2-[(1S,3S)-3-sulfonylaminocyclopentyl]phenylacetamide derivatives, zamifenacin, (2S, 3′R) 3-quinuclidinyl tropate, 3-quinuclidinyl 3-hydroxymethyl 2-phenyl alkanoates, 3-quinuclidinyl 3-hydroxymethyl 2-thienyl alkanoates, 1-azabicyclo[2.2.2]octan-3-yl 2-aryl-3-azacyclo-2-hydroxypropionates, 3-quinuclidinol esters, unsymmetrical alpha-disubstituted glycolic acids, benztropine, methscopolamine, KRP-197, and tropicamide, prodrugs of any of them, and mixtures thereof.

34. The therapeutic composition according to claim 21, wherein the muscarinic receptor antagonist is tiotropium bromide.

35. A pharmaceutical composition comprising a Cox-2 inhibitor, a muscarinic receptor antagonist, and a pharmaceutically acceptable carrier.

36. A kit comprising one dosage form comprising a Cox-2 inhibitor and a second dosage form comprising a muscarinic receptor antagonist.
